SEQUENCE LISTING (162 sequences)

SEQ ID NO: 1 - protein, 1192 aa, Aspergillus niger
  AniPYC
MAAPRQPEEAVDDTEFIDDHHDQHRDSVHTRLRANSAIMQFQKILVANRGEIPIRIFRTAHELSLQTVAVYSHEDRLSMHRQKADEAYMIGKRGQYTPVGAYLAIDEIVKIALEHGVHLIHPGYGFLSENAEFARKVEQSGMVFVGPTPQTIESLGDKVSARQLAIRCDVPVVPGTPGPVERYEEVKAFTDTYGFPIIIKAAFGGGGRGMRVVRDQAELRDSFERATSEARSAFGNGTVFVERFLDRPKHIEVQLLGDNHGNVVHLFERDCSVQRRHQKVVEIAPAKDLPADVRDRILADAVKLAKSVNYRNAGTAEFLVDQQNRYYFIEINPRIQVEHTITEEITGIDIVAAQIQIAAGATLEQLGLTQDRISTRGFAIQCRITTEDPSKGFSPDTGKIEVYRSAGGNGVRLDGGNGFAGAIITPHYDSMLVKCTCRGSTYEIARRKVVRALVEFRIRGVKTNIPFLTSLLSHPVFVDGTCWTTFIDDTPELFALVGSQNRAQKLLAYLGDVAVNGSSIKGQIGEPKLKGDIIKPVLHDAAGKPLDVSVPATKGWKQILDSEGPEAFARAVRANKGCLIMDTTWRDAHQSLLATRVRTIDLLNIAHETSHALANAYSLECWGGATFDVAMRFLYEDPWDRLRKLRKAVPNIPFQMLLRGANGVAYSSLPDNAIYHFCKQAKKCGVDIFRVFDALNDVDQLEVGIKAVHAAEGVVEATICYSGDMLNPSKKYNLPYYLDLVDKVVQFKPHVLGIKDMAGVLKPQAARLLIGSIRERYPDLPIHVHTHDSAGTGVASMIACAQAGADAVDAATDSLSGMTSQPSIGAILASLEGTEHDPGLNSAQVRALDTYWAQLRLLYSPFEAGLTGPDPEVYEHEIPGGQLTNLIFQASQLGLGQQWAETKKAYESANDLLGDVVKVTPTSKVVGDLAQFMVSNKLTAEDVIARAGELDFPGSVLEFLEGLMGQPYGGFPEPLRSRALRDRRKLDKRPGLYLEPLDLAKIKSQIRENYGAATEYDVASYAMYPKVFEDYKKFVAKFGDLSVLPTRYFLAKPEIGEEFHVELEKGKVLILKLLAIGPLSEQTGQREVFYEVNGEVRQVSVDDKKASVENTARPKAELGDSSQVGAPMSGVVVEIRVHDGLEVKKGDPIAVLSAMKMEMVISAPHSGKVSSLLVKEGDSVDGQDLVCKIVKA

SEQ ID NO: 2 - protein, 1148 aa, Brevibacillus brevis
  BbrPYC
MKKRKINRLLVANRGEIAIRIFRAATELGIRTVAVYSEQDNVSIHRFKADESYLVGAGKGPIEAYLDIESIIEIAKRNDIDAVHPGYGFLAENAEFAKRCQEEGIIFIGPSPALIDKFGDKVEARRLAIEAEIPVIPGTPEPIETLQEALLFAKEYGYPIIIKGVSGGGGRGMRIVRSQEGLQDSLDRARSEARSSFGNAKVYLERYLEQPKHIEVQILGDNHGNIVYLYERDCSVQRRHQKVVEVAPSLSLDDKLREDICQAALTLMKKAGYSNAGTVEFLLTPDKRFYFIEVNPRIQVEHTITELITGIDIVQSQIRVAEGHALSDEEIGIRAQSDIQMSGFAIQCRVTTEDAENNFLPDAGRLSAWRSGGGFGVRLDGGNGYPGAIITPFYDSLLVKISTYGASFDQAARKMLRTLREFRIRGVKTNLPFLENVVTHPDFLSGNYDTSFIDTKPELFIFPGRQDRGTKLLSYIGDTIVNGYPGLPKSEKKPHFDSPRIPRTPFTQPYPDGTKQILDKEGADGLVRWIQAQKQVLVTDTTFRDAHQSLFATRVRTYDLASIAEATGKLGSGLFSLEMWGGATFDTTMRFLQESPWERLQILRQRIPNVLFQMLLRGANGVGYTNYPDNAIHAFVKASAENGIDVFRIFDSLNWLPGMQTAIEAVRESGKVAEAAICYTGDILDPTKTKYTLAYYVNLAKELEKAGAHILAIKDMAGLLKPYAAYSLVSALKQEISIPIHLHTHDTSGNAGAMLLKAIEAGVDIVDACVSSMSGLTSQPSLNGLIASLAHTERETGLSLESFNKLSDYWEDVRPYYQGFESGMKASNTEVYVHEMPGGQYTNLEQQAKAVGLEGRWDEVKHMYAVVNQMCGDIVKVTPSSKVVGDMALFMVQNNLNEENIWEKGTRLDFPDSVIQFFQGYLGQPPGGFPKKLQELVLKGRDAFTARPGELLAPIDFTQVAAELEAKIGREPSHLDVLSYIMYPQVYLQFEQRLKEYGDLSVLNTGTFFYGLRPGEETAITIERGKTLIIKLVAVGELHPDGRRIIYFELNGQPREIFIRDQSAKVSELIRRKAEAQNPAHLGASMPGKVLKVLVAEGDKVRKGEHLLVSEAMKMETTIQAPLDGKIKAVYVKAGEAIQTGDLLIEME

SEQ ID NO: 3 - protein, 1173 aa, Debaryomyces hansenii
  DhaPYC
MSDGNEHKINQMRRASTVMGPMNKILVANRGEIPIRIFRTAHELSMQTVAIFSHEDRLSMHRLKADESYVIGKKGQFSPVQAYLQIDEIINIAKEHGVNMIHPGYGFLSENSEFARKVEEAGISWIGPTHKTIDAVGDKVSARTLAIQNGVPVVPGTPGPIADVEEAVKFVEKHGLPVIIKAAFGGGGRGMRVVREGDSVGEAFERAVSEAKTSFGNGTCFIERFLDKPKHIEVQLLADNYGNVIHLFERDCSVQRRHQKVVEVAPAKTLTKSVRDAILTDAVKLARSANYRNAGTAEFLVDEQDRHYFIEINPRIQVEHTITEEITGVDIVAAQIQIAAGASLKQLGLLQDKITTRGFAIQCRITTEDPSKNFQPDTGKIEVYRSAGGNGVRLDGGNGFAGSIISPHYDSMLVKCSCSGSTYEIVRRKMLRALIEFRIRGVKTNIPFLLAMLTNETFISGDCWTTFIDDTPSLFQMIRSQNRATKILSYLGDLIVNGSSIKGQIGEPKLNTEALIPDLHDPKTGIKIEIHNEVAPRGWRQVLLEQGPEKFAQKVREFNGTLLMDTTWRDAHQSLLATRVRTIDLLNIAPTTAVALNGCFALECWGGATFDVCMRFLYEDPWSRLRQLRKLVPNIPFQMLLRGANGVAYSSLPDNAIDQFVLQAKENGVDIFRVFDALNDLEQLKVGIDAVKKAGGVIEATVCYSGDMLKPGKKYNLEYYLGVVDDIVKLGTHFLGIKDMAGTLKPKAAKILIGAIRERYPKLPIHVHTHDSAGTGVASMTEAAKAGADVVDAASNSMSGMTSQPSINAILASFEGEVESGLQASLVRELDNYWAQMRLLYSCFEADLKGPDPEVYQHEIPGGQLTNLLFQAQQLGLGSKWMLTKEAYKTANKLLGDIVKVTPTSKVVGDLAQFMVSNNLSEEDVTKLASELDFPSSVLDFMEGLMGTPYGGFPEPLRTNILGTKRVKLNNRPGLNLPPIDFAAVKEELVSRYGSNINECDIASYVMYPKVYEDFRELSEKYGDLSVLPTRFFLKACNINEEIVVDIEKGKTLIIKLLAIGEISQQTGTREVFFELNGEMRSVTVDDKTVSIETKTRPKATSPNEVGAPMAGVVVEIRSKVGNEVKKGDPIAVLSAMKMEMVISSPVSGKIGEILIKEGDSVDANDLIASVVK

SEQ ID NO: 4 - protein, 1173 aa, Kluyveromyces lactis
  KlaPYC
MSSQLAGLRDNSNLMGEKNKLLVANRGEIPIRIFRTAHELSMKTVAIYSHEDRLSMHRLKADEAYVIGEQGKYTPVGAYLAIDEIINIAKSHGVNFIHPGYGFLSENSEFAEKVAASGITWVGPPAAVIDSVGDKVSARNLAEKADVPVVPGTPGPIETVEEAQAFVDKYGFPVIIKAAFGGGGRGMRVVREGDDISDAFQRAKSEAITAFGNGTCFIERFLDKPKHIEVQLLADNYGNVVHLFERDCSVQRRHQKVVEVAPAKTLPEEVRDAILTDAVKLAKEAGYRNAGTAEFLVDNQNRHYFIEINPRIQVEHTITEEITGIDIVAAQIQIAAGATLEQLGLMQDKITTRGFAIQCRITTEDPSKNFQPDTGRIDVYRSAGGNGVRLDGGNAFAGSVISPHYDSMLVKCSCSGSTYEIVRRKMLRALIEFRIRGVKTNIPFLLTLLTHPVFKSGEYWTTFIDDTPQLFEMVSSQNRAQKLLHYLADLAVNGSSIKGQIGLPKLTTHPTIPHLHNEDGSLVDVSAKPPAGWRDVLLDYGPEEFAKQVRKFNGTLLMDTTWRDAHQSLLATRVRTYDLAAIAPTTAHAMSGAFALECWGGATFDVSMRFLHEDPWERLRTLRKLVPNIPFQMLLRGANGVAYSSLPDNAIDHFVKQAKDNGVDIFRVFDALNDLEQLKVGVDAVKKAGGVVEATICYSGDMLAPGKKYNLDYYLDITEKIVNSGTHILGIKDMAGTLKPSAARLLIGSIRAKYPELPIHVHTHDSAGTGVASMAACALSGADVVDVATNSMSGLTSQPSINALLAALDGEIDNNVNVGYVRELDAYWAEMRLLYSCFEADLKGPDPEVYVHEIPGGQLTNLLFQAQQLGLGEKWAETKRAYREANLLLGDLVKVTPTSKVVGDLAQFMVTNKLTSDDVRRLASSLDFPDSVMDFFEGLIGQPYGGFPEPLRTDVLRNKRRKLTQRPGLELAPFELEKIKEDLSTRFSDIDECDVASYNMYPKVYEDFRKIKEKYGDLSVLPTKNFLSPPVIGEEIVVTIEQGKTLIIKPQAIGDLNKETGIREVYFELNGELRKVSVADRSQKVETVSKPKADAHDPFQIGSPMAGVVVEVKVHKGSLIAKGQPVAVLSAMKMEMVISSPADGQVKEVLVKDGENVDASDLLVVLEEAPAKE

SEQ ID NO: 5 - protein, 1174 aa, Lachancea thermotolerans
  LthPYC
MSKLAGLRDSSNLLGEKNKLLVANRGEIPIRIFRSAHELSMKTVAIYSHEDRLSMHRLKADEAYMIGKEGKYTPVGAYLAIDEILKIAKEHNVDFIHPGYGFLSENSEFAQKVEAAGITWIGPSAEVIESVGDKVSARNLAAKADVPVVPGTPGPIDSVEEAQAFVEEYGYPVIIKAAYGGGGRGMRVVREGDDIADAFQRATSEAKTAFGNGTCFIERFLNKPKHIEVQLLADGYGNVVHLFERDCSVQRRHQKVVEVAPAKTLPKDVRDAILTDAVKLAKVAGYKNAGTAEFLVDDQNRHYFIEINPRIQVEHTITEEITGIDIVAAQIQIAAGASLQELGLLQDKIITRGFAIQCRITTEDPAKNFQPDTGRIEVYRSAGGNGVRLDGGNAYAGSIISPHYDSMLVKCSCSGSTYEIVRRKMLRALIEFRIRGVKTNIPFLLTLLTNPVFIDGSYWTTFIDDTPELFKMVSSQNRAQKLLHYLADLAVNGSSIKGQMGLPKLHTQPSVPTLHDSNGEVIDVLTTPPPSGWRQVLLEQGPAQFAKAVRHYKGTLLMDTTWRDAHQSLLATRVRTYDLAAIAPTTAHALSGAFALECWGGATFDVAMRFLHEDPWERLRILRKLVPNIPFQMLLRGANGVAYSSLPDNAIDHFVEQAKENGVDIFRVFDALNDLEQLKVGVDAVKKANGVVEATICYSGDMLQPGKKYNLDYYLEITDKIVQMGTHILGIKDMAGTLKPSAAKLLIGSIRAKYPDLPIHVHTHDSAGTAVASMAACAFAGADVVDVATNSMSGMTSQPSINALLASLDGEIDTGVNVNMVRELDAYWAQMRLLYSCFEADLKGPDPEVYEHEIPGGQLTNLLFQAQQLGLGEKWAETKRAYRDANYLLGDLVKVTPTSKVVGDLAQFMVSNKLTPDDVRRLASSLDFPDSVMDFFEGLIGQPYGGFPEPLRSDVLKNKRRKLTCRPGLELAPFDLAGIKEDLQDRFGDIDECDVASYNMYPKVYEDFRKMRETYGDLSVLPTKNFLSPPTVGEEIVVTIEKGKTLIIKPQAIGELNKETGLREVYFDLNGELRKVSVIDKSQKVDTLSKPKADAHDPFQIGAPMAGVIIEVKVHKGSLVKKGQPIAVLSAMKMEMVISSPTDGQVKDVLVKDGENVDASDLLVFLEESVPPKE

SEQ ID NO: 6 - protein, 1179 aa, Lodderomyces elongisporus
  LelPYC
MTSIASSNTQDGRINQMRRDSTVLGPMNKILVANRGEIPIRIFRTAHELSMQTVAIYSHEDRLSMHRLKADESYAIGKKGQFTPVGAYLQIDEIIDIAKKHNVNMIHPGYGFLSENSEFAKKVEQNGIVWIGPSHKTIDAVGDKVSARTLAIENDVPVVPGTPGPIDDVSDARKFVEKYGYPVIIKAAFGGGGRGMRVVREGDDIEDAFKRATSEAKTAFGNGTCFIERFLDKPKHIEVQLLADNYGNVIHLFERDCSVQRRHQKVVEIAPAKNLPREVRDAILTDAVKLAKSANYRNAGTAEFLVDEQNRHYFIEINPRIQVEHTITEEITGVDIVAAQIQIAAGASLQQLGLLQDKITTRGFAIQCRITTEDPAKNFQPDTGKIEVYRSAGGNGVRLDGGNGFAGSIISPHYDSMLVKCSCSGSTFEIARRKMIRALIEFRIRGVKTNIPFLLALLTNQTFVEGDCWTTFIDDTPSLFQMISSQNRATKLLLYLADLYVNGSSIKGQIGYPKLDTEALIPELHEPRTGIAIDVNHTPPPRGWRQVLLEEGPAKFAKKVREFKGCLITDTTWRDAHQSLLATRVRTIDLLNIAPTTAFALNGAFSLECWGGATFDVCMRFLYEDPWVRLRQLRALVPNIPFQMLLRGANGVAYSSLPDNAIDQFVKEAKENGVDIFRVFDALNDLEQLKVGVDAVKKAGGVVEATVCYSGDMLKPGKKYNLEYYLNFVDEVVKMGTHFLGIKDMAGTLKPAAAKLLVGEIRKRHPDLPIHVHTHDSAGTGVASMTECAKAGADVVDAASNSMSGMTSQPSISAILASFEGSIESGLSEQLVRELDEYWAQMRLLYSCFEADLKGPDPEVYSHEIPGGQLTNLLFQAQQLGLGTKWLETKKAYRIANKILGDLVKVTPTSKVVGDLAQFMVSNSLTEEDINRLAGELDFPDSVYDFMEGLMGQPYGGFPEPLRTNMLGNKRQKLTQRPGLSLPPVKFDEIKQELQSRYGTQVSETDIASYVMYPKVYEAYRKQVEKYGDLSVLPTRYFLKPANIGQEIVVDIEQGKTLIIKLLAVGELSEKTGSREIFFELNGEMRSVTVEDKTASVETKTRPKAQQPNDVGAPMSGVVIEVRTHKHQEVKKGDPIAVLSAMKMEMIISAPVSGVVGDILVKEGDSVDANDLITSILKH

SEQ ID NO: 7 - protein, 1174 aa, Saccharomyces cerevisiae; identical to SEQ ID NO: 5
  ScePYC

SEQ ID NO: 8 - protein, 1216 aa, Candida orthopsilosis
  CorPYC
MIFINSSSISTKSPLNSISLSSTQLLFFCQIYSPPITMTSLQPTATQDGQINQMRRASSVLGPMNKILVANRGEIPIRIFRTAHELSMQTVAIYSHEDRLSMHRLKADESYVIGKKGQYSPVGAYLQIDEIIDIAKQHNVNMIHPGYGFLSENSEFARKVEENGILWVGPSYKTIDAVGDKVSARTLAIENDVPVVPGTPGPIDDVTDARKFVEKYGFPVIIKAAFGGGGRGMRVVREGDDIEDAFKRATSEAKTAFGNGTCFIERFLDKPKHIEVQLLADNYGNVIHLFERDCSVQRRHQKVVEIAPAKNLPKEVRDAILTDAVKLAKSANYRNAGTAEFLVDEQNRHYFIEINPRIQVEHTITEEITGVDIVAAQIQIAAGASLQQLGLLQDKITTRGFAIQCRITTEDPAKNFQPDTGKIEVYKSAGGNGVRLDGGNGFAGSVISPHYDSMLVKCSCSGSTFEIARRKMLRALIEFRIRGVKTNIPFLLALLTNETFIKGDCWTTFIDDTPSLFQMVSSQNRATKLLSYLADLYVNGSSIKGQIGYPKLNEEALIPVLHDPKTGIPIDVAHIPPPRGWRQVLLEEGPDAFAKKVRQYNGTLITDTTWRDAHQSLLATRVRTIDLLNIAPTTAFALNGAFSLECWGGATFDVCMRFLYEDPWDRLRKLRALVPNIPFQMLLRGANGVAYSSLPDNAIDQFVKEAKGNGVDIFRVFDALNDLEQLKVGIDAVKKAGGVVEATVCYSGDMLKPGKKYNLEYYLSVVDEVVKMGTHFLGIKDMAGTLKPAAARLLVGEIRNRYPELPIHVHTHDSAGTGVASMTECAKAGADVVDAASNSMSGLTSQPSISAILASFEGSIDTGLSESLVRELDTYWAQMRLLYSCFEADLKGPDPEVYSHEIPGGQLTNLLFQAQQLGLGEKWLQTKETYKIANKILGDLVKVTPTSKVVGDLAQFMVSNSLTEEDVNRLASELDFPDSVLDFMEGLMGKPYGGFPEPLRTNMLGNKRQKLDKRPGLYLKPVDFAAIRKELISRYGSQIDETDVASYVMYPKVFEAFRKQVEKYGDLSVLPTRFFLKPANIGEEIVVDIEKGKTLIIKLLAVGEISEKTGTREVFFELNGEMRSVSVEDNTVSVETKTRPKASAPNDVGAPMAGVVIEVRTHKHQEVKKGDPIAVLSAMKMEMVISAPVSGLVDDLLVREGDSVDANDLITSILKA

SEQ ID NO: 9 - protein, 1180 aa, Candida tropicalis
  CtrPYC
MSVPVQKANSQDTRISQMRRDSTVLGPMNKILVANRGEIPIRIFRTAHELSMQTVAIYSHEDRLSMHRLKADESYVIGKKGQFSPVGAYLQIDEIINIAKKHNVNMIHPGYGFLSENSEFARKVEENGIIWIGPSYKTIDAVGDKVSARTLAIENDVPVVPGTPGPIETVEEAKAFVDKYGLPVIIKAAFGGGGRGMRVVREGDDIEDAFKRATSEAKTAFGNGTCFIERFLVKPKHIEVQLLADNYGNVIHLFERDCSVQRRHQKVVEIAPAKSLPKHVRDAILTDAVKLAKSANYRNAGTAEFLVDAQDRHYFIEINPRIQVEHTITEEITGVDLVAAQIQIAAGASLQQLGLLQDKITTRGFAIQCRITTEDPTKNFQPDTGKIEVYRSAGGNGVRLDGGNGFVGSIISPHYDSMLVKCSCSGSTYEIARRKMLRALIEFRIRGVKTNIPFLLALLTNDIFIKGDCWTTFIDDTPSLFQMISSQNRATKMLSYLADLVVNGSSIKGQVGYPKLETEPVVPDIHEPKTGIVIDVNNTPAPRGWRQVLLEEGPEAFAKKVRAFKGTLITDTTWRDAHQSLLATRVRTIDLVNIASTTAFALNGAFSLECWGGATFDVCMRFLYEDPWARLRKLRALVPNIPFQMLLRGANGVAYSSLPDNAIDQFVKEAKDNGVDIFRVFDALNDLEQLKVGIDAVKKAGGVVEATVCYSGDMLKPGKKYNLEYYLNVVDEIVKLGTHFLGIKDMAGTLKPAAAKILIGEIRSRYPDLPIHVHTHDSAGTGVASMTQCAVSGADVVDAASNSMSGMTSQPSISALLASFEGSIDTGLSESMVRELDNYWAQMRLLYSCFDADLKGPDPEVYQHEIPGGQLTNLLFQAQQLGLGSQWVQTKEAYKVANSILGDLVKVTPTSKVVGDLAQFMVSNTLTEEDVNRLAAELDFPDSVLDFFQGLMGTPYGGFPEPLRTNILGSKRQKLNERPGLTLPPVDFVGIREELSSRYGGQITETDIASYVMYPKVFEQFRNIIDKYGDLSVLPTKYFLKPCAIGEELSVDIEQGKTLIIKLLAVGDISDKTGNREVFFELNGEMRSVSVEDKAAAVETRVKPKASSPNDVGAPMAGVVIEIRAHRHQEVKKGDPIAVLSAMKMEMVISSPCDGEVGEIVIHEGDSVDANDLITNIIQH

SEQ ID NO: 10 - protein, 1176 aa, Naumovozyma castellii
  NcaPYC
MSINKKLNGLRDNFNLLGTRNKILVANRGEIPIRIFRSAHELSMQTVAIYSHEDRLSTHRLKADESYVIGEPHQFTPVGAYLAIDEIINIAKRHGVDYIHPGYGFLSENSEFADKVAKAGITWIGPPASVIDSVGDKVSARNLATKANVPTVPGTPGPIRTVQDAQDFVNEYGYPVIIKAAFGGGGRGMRVVNEGDDLADAFQRASSEALTAFGDGTCFVERFLNKPKHIEVQLLADTHGNVIHLFERDCSVQRRHQKVVEVAPAKTLPREVRDAILTDAVKLAKECGYQNAGTAEFLVDDQNRHYFIEINPRIQVEHTITEEITGIDIVAAQIQIAAGASLEQLGLLQDKITTRGFAIQCRITTEDPAKNFQPDTGRLEVYDSAGGNGVRLDGGNAYAGAIISPHYDSMLVKCSCSGSTYEVVRRKMIRALIEFRIRGVKTNIPFLLTLLTHPVFIAGDYWTTFIDDTPQLFKMVSSQNRAQKLLHYLADLAVNGSSIKGQVGIPQLKKVPDIPHIHDAQGNVIDVTNVPPPAGWRQVLLEQGPEEFAKQVRQFQGTLLMDTTWRDAHQSLLATRVRTHDLAKIAPTTAHALASAFALECWGGATFDVAMRFLHEDPWKRLRTLRKLVPNIPFQMLLRGANGVAYSSLPDNAIDHFVKQAKDNGVDIFRVFDALNDLDQLKVGVDAVKKAGGVVEATVCYSGDMLQPGKKYNLDYYLEVTEKIVQMGTHILGIKDMAGTMKPAAARLLIGSIRAKYPDLPIHVHTHDSAGTAVASMSAAAYAGADVIDVATNSMSGLTSQPSINALLASLDGEINTSINVQHVRELDAYWAEMRLLYSCFGADLKGPDPEVYEHEIPGGQLTNLLFQAQQLGLGEQWTETKRAYKEANMLLGDIVKVTPTSKVVGDLAQFMVSNKLTSEDVKRLANSLDFPDSVMDFFEGLMGQPYGGFPEPLRSDILRNKRRKLTCRPGLELVPFELNNIKEDLQTRFGDINECDIASYNMYPKVYEDFQKMKEKYGDLSVLPTKSFLAPAEIGEEILVTIQQGKTLIIKLQAIGDLNKETGKREVFFELNGEMRKISVTDKSQKVETVAKPKADTHDPFQIGAPMAGVIVEVKVHKGSLVKKGEAVAVLSAMKMEMVISSPADGLVKEVLVNESENVDASDLLVLLEESTPPS

SEQ ID NO: 11 - protein, 1178 aa, Naumovozyma dairenensis
  NdaPYC
MSNKKFNGIRDNFNLLGPKNKILVANRGEIPIRIFRTSHELSMKTVAIYSHEDRLSTHRLKADESYVIGEPNQFTPVGAYLAIDEIINIAKKHNVDFIHPGYGFLSENSEFADKVAKNGIAWVGPPASVIDSVGDKVSARHLAERANVPTVPGTPGPIKSAKEAEEFVAKYGFPVIIKAAFGGGGRGMRVVREGDDIADAFQRASSEAVTAFGNGTCFVERFLNKPKHIEVQLLADNYGNVIHLFERDCSVQRRHQKVVEVAPAKTLPRDIRNAILTDAVKLAKECGYKNAGTAEFLVDDQNRHYFIEINPRIQVEHTITEEITGIDIVAAQIQIAAGASLEQLGLLQDKITTRGFAIQCRITTEDPAKNFQPDTGRIEVYGSAGGNGVRLDGGNAYAGAIISPHYDSMLVKCSCSGSTYEVVRRKMIRALIEFRIRGVKTNIPFLLTLLTHPVFISGQYWTTFIDDTPQLFQMVSSQNRAQKLLHYLADIAVNGSSIKGQVGIPRLTKIPDIPPLYDTNNNKIDVLKTPPPAGWRQVLLEKGPVEFAKQVRQFQGTLLMDTTWRDAHQSLLATRVRTYDLATIAPTTAHALSGAFALECWGGATFDVAMRFLHEDPWARLRTLRKLVPNIPFQMLLRGANGVAYSSLPDNAIDHFVKQAKDNGVDIFRVFDALNDLDQLKVGVDAVKKAGGVVEATLCYSGDMLAKGKKYNLDYYLEVTDKIVQMGTHFLGIKDMAGTMKPAAAKLLIGSIRAKYPDLPIHVHTHDSAGTAVSSMAAAAVSGADVVDVAINSMSGLTSQPSINALLASLDGEIDTGINVQHVRDLDAYWAEMRLLYSCFGADLKGPDPEVYQHEIPGGQLTNLLFQAQQLGLGEQWAETKRAYREANHLLGDIVKVTPTSKVVGDLAQFMVSNKLTSDDVKRLANSLDFPDSVMDFFEGLMGQPYGGFPEPLRSDILRNKRRKLTSRPGLELAPFDLNTIREDLQTRFGDDIDECDVASYNMYPKVYEDFQKIRETYGDLSVLPTKNFLAPAEIGEEIEIVIEQGKTLIVKLQAIGDLNKKTGIREVYFELNGEMRKIGTLDRSQKVETVAKPKADGHNPFQIGAPMAGVIVEVRVHKGSLVKKGEAVAVLSAMKMEMIISSPTDGQVKDVLVNDGENVEASDLLVLLEETEIPEEA

SEQ ID NO: 12 - protein, 1178 aa, Saccharomyces kudriavzevii
  SkuPYC
MSQKKFAGLRDNFNLLGEKNKILVANRGEIPIRIFRTAHELSMQTVAIYSHEDRLSTHKQKADEAYVIGEAGQYTPVGAYLAIDEIISIAQKHQVDFIHPGYGFLSENSEFADKVAKAGITWIGPPAEVINSVGDKVSARNLAARANVPTVPGTPGPIESVQEALDFVGEYGYPVIIKAAFGGGGRGMRVVREGDDVADAFQRATSEARTAFGNGTCFVERFLVKPKHIEVQLLADNHGNVVHLFERDCSVQRRHQKVVEVAPAKTLPREVRDAILTDAVKLAKECGYRNAGTAEFLVDNQNRHYFIEINPRIQVEHTITEEITGIDIVAAQIQIAAGASLPQLGLFQDKITTRGFAIQCRITTEDPAKNFQPDTGRIEVYRSAGGNGVRLDGGNAYAGTIISPHYDSMLVKCSCSGSTYEIVRRKMIRALIEFRIRGVKTNMPFLLTLLTHPVFIDGTYWTTFIDDTPQLFQMVSSQNRAQKLLHYLADVAVNGSSIKGQIGSPKLGSNPSIPHLHDSQGNVINVTKTTPPSGWRQVLLEKGPAEFAKQVRQFNGTLLMDTTWRDAHQSLLATRVRTHDLATIAPTTAHALAGAFALECWGGATFDVAMRFLHEDPWERLRKLRALVPNIPFQMLLRGANGVAYSSLPDNAIDHFVKQAKDNGVDIFRVFDALNDLDQLKVGVDAVKKAGGVVEATVCFSGDMLQPGKKYNLDYYLEIAEKIVKMGTHILGIKDMAGTMKPAAAKLLIGSLRAKYPDLPIHVHTHDSAGTAVASMAACALAGADVVDVAINSMSGLTSQPSINALLASLEGNIDTGINVEHVRELDAYWAEMRLLYSCFEADLKGPDPEVYQHEIPGGQLTNLLFQAQQLGLGEQWAETKRAYREANYLLGDIVKVTPTSKVVGDLAQFMVSNKLTPDDVKRLANSLDFPDSVMDFFEGLIGQPYGGFPEPFRSDVLRNKRRKLTCRPGLELEPFDLEKIREDLQNRFGDINECDVASYNMYPRVYEDFQKMRETYGDLSVLPTRSFLSPLETDEEIEVIIEQGKTLIIKLQAVGDLNKKTGEREVYFDLNGEMRKIRVVDRSLKVATVTKSKADMHDPLHIGAPMAGVIVEVKVHKGSLIKKGQPVAVLSAMKMEMIISSPSDGQVKEVFVSEGENVDSSDLLVLLEDQVPAETKA

SEQ ID NO: 13 - protein, 1185 aa, Schizosaccharomyces pombe
  SpoPYC
MTSKYDALLHNQSTNTNPFSKLQDRSSLLGEKFTKVLVANRSEIAIRVFRTAHELSMHTVAIYSYEDRLSMHRQKADESYPIGKVGQYSPVGAYLAIDEIVSIAKRTGANLVHPGYGFLSENAEFARKVNEAGMQFVGPSPEVIDSLGDKTKARAIAIRCGVPVVPGTPGPVEHYEEAEAFVKEYGLPVIIKAAMGGGGRGMRVVRSADTLKESFERARSEALASFGDGTVFIERFLDKPKHIEIQLMADKAGNVIHLHERDCSVQRRHQKVVEIAPAKDLDPKIRQALYDDAIKIAKEVKYCNAGTAEFLLDQKGRHYFIEINPRIQVEHTITEEITGVDIVSAQLHVAAGFTLPEIGLTQDKISTRGFAIQCRVTTEDPNNGFAPDIGKIEVYRSAGGNGVRLDGANGFAGSVITPHYDSMLVKCTCHDATYEYTRRKMIRSLIEFRVRGVKTNIPFVLRLLMHDTFIQGNCWTTFIDDTPELFQLYRSRNRAQKLLAYLGDLAVNGSSIKGQNGEPALKSEIVMPVLLDSTGNQIDVSHPSEKGWRKLLLDNGPAAFAKAVRNHKRGLIMDTTWRDAHQSLLATRVRTIDLVNIAPYTSHALASAYSLEMWGGATFDVSMRFLHECPWDRLRRLRKLVPNIPFQMLLRGANGLCYSSLPDNVIYFFCEQAKKNGIDIFRVFDALNDVNNLSLGIDAAKRAGGVVEATMCYSGDMLNPKKKYNLDYYVNLVDKMVEMGIHILGIKDMAGVMKPKAARLLISAIREKHPELPIHVHTHDSAGTAVASMAAALEAGADVVDVATDSMSGLTSQPSFGAVLASVDGTDKQLEFDNNQLREIDSYWAQMRLLYSPFESEIKGTDSDVYNHEIPGGQLTNLKFQATSLGLGTQWAETKKAYIEANKLLGDIIKVTPTSKVVGDLAQFMVQNKLSAEDVENRATTLDFPASVLDFFQGLMGQPYGGFPEPLRTNVLKGRRQPLTDRPGKFLPAADFDAIRKLLSEKFGVSSDCDIAAYTQFPGVFEEYRQFVDRYGDLTTVPTKFFLSRPEMNEEMHVEIDQGKTLIVKFVALGPLNPRTGQREVYFELNGENRHVTVEDKKAAIETVTRPRADPGNPGHVAAPMSGTIVEIRVKEGAKVKKGDIIAVLSAMKMEIVISAPHSGVLKSLAVVQGDSVNGGDLCAVLEHE

SEQ ID NO: 14 - protein, 1175 aa, Tetrapisispora blattae
  TblPYC
MTNKKFSGLRDNFSLLGEKNKVLVANRGEIPIRIFRTAHELSMRTVAIYSHEDRLAMHRLKADESYVIGAENQYTPVGAYLAIDEIINIAKKHNVDFIHPGYGFLSENSEFARKVTESGITWIGPPAAVIDSVGDKVSARNLAAKVNVPTVPGTPGPIDTVEEAKAFVEKHGFPVIIKAAFGGGGRGMRVVREGDDIADAFQRATSEARTAFGNGTCFIERFLDHPKHIEVQLLADNYGNVVHLFERDCSVQRRHQKVVEVAPAKTLPREVRDSILTDAVKLAKEAGYTNAGTAEFLVDNLGRHYFIEINPRIQVEHTITEEITGIDIVAAQIQIAAGASLEDLGLLQDRITYRGFAIQCRITTEDPSKNFQPDTGRLEVYRSAGGNGVRLDGGNAYVGATISPHYDSMLVKCSCSGSTYEIVRRKMLRSLIEFRIRGVKTNIPFLLTLLTHPVFVSGDYWTTFIDDTPQLFQMVSSKNRAQKLLHYLADLAVNGSSIKGQIGLPKLATRPDIPALHDKNGEVINVTNAEPPSGWRKALLEKGPKEFAKQVREFNGCLIMDTTWRDAHQSLLATRLRTYDLAAIAPTTSFALAGAFALECWGGATFDVAMRFLHEDPWERLRTLRKLVPNIPFQMLLRGANGVAYSSLPDNAINHFVKQAKDNGVDIFRVFDSLNDVEQLKVGIEAVKLAGGVVEATMCYSGDMLQPGKKYNLDYYLELSEKIVGLGTHILGIKDMAGTMKPAAAKLLISSLRKKYPDLPIHVHSHDSAGTAVTSMVACAKYGADVVDVAINSMSGLTSQGSINALLASLDGDIETNINAQHVRELDAYWAEMRLLYSCFEADLKGPDPEVYEHEIPGGQLTNLLFQAQQLGLGEKWKETKRAYREANYLLGDIVKVTPTSKVVGDLAQFMVTNKLTSDDVRRLANSLDFPDSVMDFFEGLLGQPYGGFPEPLRTDILKGKRKKMTSRPGLELQPFDIAAIKEDLQNRFGDIDECDVASYNMYPKVYEDFQKIREKYGDLSVLPTKNFLAPPVTGEEIEVTIEQGKTLIIKCQAVGDLNKATGTREVYFELNGELRKIPVVDRSLKVDIVAKPKADTHDPYQIGAPMAGVIVEVKVHKGSLVKKGQPVAVLSAMKMEMVISSPSDGLVKNVTVADGENVDASDLLITLEDPEPPK

SEQ ID NO: 15 - protein, 1177 aa, Torulaspora delbrueckii
  TdePYC
MSKNKRFAGLRDNFSLLGEKNKLLVANRGEIPIRIFRSAHELSMRTVAIYSHEDRLSMHKLKSDEAYVIGEEGKYTPVGAYLAIDEIIEIAKQHNVDFIHPGYGFLSENSEFAKKVADNGITWIGPPADVIDAVGDKVSARNLALKADVPTVPGTPGPIENVQEAKAFVEKYGYPVIIKAAFGGGGRGMRVVREGDDIEDAFQRATSEARTAFGNGTCFIERFLVKPKHIEVQLLADNYGNVVHLFERDCSVQRRHQKVVEVAPAKTLPVEVRNAILTDAVKLAHTAGYRNAGTAEFLVDNQNRHYFIEINPRIQVEHTITEEITGIDIVAAQIQIAAGASLEQLGLMQDRITTRGFAIQCRITTEDPSKNFQPDTGRLEVYRSAGGNGVRLDGGNAFAGAIISPHYDSMLVKCSCSGSTYEIVRRKMIRALIEFRIRGVKTNIPFLLTLLTNPVFVNGDYWTTFIDDTPQLFQMISSQNRAQKLLHYLADLAVNGSSIKGQLGVPKLTTHPTIPHLHDAQGEIINVNSAAPPAGWRQILLDYGPKEFAKQVRDFDGTLIMDTTWRDAHQSLLATRVRTYDLAAIAPTTAHALSGAFALECWGGATFDVAMRFLHEDPWERLRILRKLVPNIPFQMLLRGANGVAYSSLPDNAIDHFVKQAKENGVDIFRVFDALNDLEQLKVGVDAVKKAGGVVEATVCYSGDMLQPGKKYNLDYYLEVTGKIVEMGTHILGIKDMAGTMKPSAARLLIGSIRAKYPDLPIHVHTHDSAGTAVASMAACALAGADVVDVATNSMSGLTSQPSMNALLASLDGQINTNIDVQHTRELDAYWAEMRLLYSCFEADLKGPDPEVYEHEIPGGQLTNLLFQAQQLGLGEKWAETKRAYKEANYLLGDLVKVTPTSKVVGDLAQFMVSNKLTSGDVKRLASSLDFPDSVMDFFEGLIGQPYGGFPEPLRTDILKNKRRKLTCRPGLELAPFDLEKIREDLQDRFGDIDECDVASYNMYPKVYEDFHKIKERYGDLSVLPTKNFLSPPKIGEEIEVTIEKGKTLIIKHQAVGDLNKETGVREVYFELNGELRKIPVVDKSQKVESVAKPKADGHDPLQVGAPMAGVIVEVKVHKGSLVKKGEPVAVLSAMKMEMVISAQADGQVKEVYINDGDNVDASDLLVVLEEAVPAEN

SEQ ID NO: 16 - protein, 1177 aa, Zygosaccharomyces rouxii
  ZroPYC
MSTKKFSGLRDNFSLLGEKNKILVANRGEIPIRIFRSAHELSMRSVAVYSHEDRLSMHRLKADEAYVIGEEGKYTPVGAYLAIDEIIAIAKKHNVDFIHPGYGFLSENSEFAKKVEANGITWIGPPAEVIDSVGDKVSARNLAAKAHVATVPGTPGPIESVDEAIEFTKKYGFPVIIKAAFGGGGRGMRVVREGDDVADAFQRASSEARSSFGNGTCFMERFLVKPKHIEVQLLADNYGNVVHLFERDCSVQRRHQKVVELAPAKALPVETRNAILTDAVKLAKTAGYRNAGTAEFLVDNENRHYFIEINPRIQVEHTITEEITGIDIVAAQIQIAAGASLDQLGLLQDRITTRGFAIQCRITTEDPSKNFQPDTGRIEVYRSSGGNGVRLDGGNTYAGAVISPHYDSMLVKCSCSGSTYEIVRRKMIRALVEFRIRGVKTNIPFLLTLLTHPTFVQGQFWTTFIDDTPQLFQMISSQNRAQKLLHYLADLAVNGSSIKGQVGLPKLKTHPTVPRLHGSNGEVIDVLATPPPSGWRQVLLEHGPEEFAKRVRQFNGCLIMDTTWRDAHQSLLATRVRTYDLAAIAPTTAHALSGAFSLECWGGATFDVAMRFLHEDPWERLRILRRLVPNVPFQMLLRGANGVAYSSLPDNAIDHFVKQAKENGVDIFRVFDALNDLDQLKVGVDAVKKAGGVVEATVCYSGDMLQPGKKYNLDYYLEVTEQIVKMGTHFLGIKDMAGTMKPAAAKLLVGSIRAKYPNLPIHIHTHDSAGTGVASMAACAVSGADVVDVAVNSMSGLTSQPCMNALLASLDGEINTGINANYARELDAYWAEMRLLYSCFDTDLKGPDPEVYEHEIPGGQLTNLLFQAQQLGLGEKWIETKKAYKEANYLLGDLVKVTPTSKVVGDLAQFMVSNKLTSDDVRRLANSLDFPDSVMDFFEGLIGQPYGGFPEPLRSDVLRNKRRKLTNRPGLDLAPFDLEAVREDLQDRFGDTDECDVASYNMYPKVYEDFQKIKEQFGDLSVLPTKNFLSPPGIGEEIEVTIEQGKTLIIKHQAVGDLNKETGMREVYFELNGELRKIPVVDRSQKVEIVSKPKADGHDPYQVGAPMAGVIVEIKVHKGSLIKKGQPVAVLSAMKMEMVISAQTDGQVKEVLANGGDNVDASDLLVTLEDAVPPKEN

SEQ ID NO: 17 - protein, 294 aa, Archaeoglobus fulgidus
  AfuMDH
MKLGFVGAGRVGSTSAFTCLLNLDVDEIALVDIAEDLAVGEAMDLAHAAAGIDKYPKIVGGADYSLLKGSEIIVVTAGLARKPGMTRLDLAHKNAGIIKDIAKKIVENAPESKILVVTNPMDVMTYIMWKESGKPRNEVFGMGNQLDSQRLKERLYNAGARNIRRAWIIGEHGDSMFVAKSLADFDGEVDWEAVENDVRFVAAEVIKRKGATIFGPAVAIYRMVKAVVEDTGEIIPTSMILQGEYGIENVAVGVPAKLGKNGAEVADIKLSDEEIEKLRNSAKILRERLEELGY

SEQ ID NO: 18 - protein, 326 aa, Congregibacter litoralis
  CliMDH
MKAPVRVTVTGAAGQIGYALLFRIASGAMLGDDQPVILHLLDITPAMDALEGVRMELDDCAFPLLAGIVCTDDPNVGFKDADYALLVGARPRGPGMERKDLLEANAAIFSVQGKAINDHASRDIRVLVVGNPANTNALITQRNAPDIDPRNFTAMMRLDHNRAKTQIAQKLDAPVTAVSTMTVWGNHSATQYPDLHHSTVNGKVAVDAVEQQWYEDEFIPTVQQRGAAIIKARGASSAASAANAAIDHMRDWALGTPEGDWVSMGVYSDGSYGIAEGLIYSFPCRCSGGDWSIVQGLEINDFSRGRMQATEQELTEERDAVADLLP

SEQ ID NO: 19 - protein, 328 aa, Delftia acidovorans
  DacMDH
MSKKPVRVAVTGAAGQIGYALLFRIASGEMLGKDQPVILQLLEIPDEKAQNALKGVIMELEDCAFPLLAGIEAHSDPLQAFKDTDYALLVGARPRGPGMERADLLAANAQIFTAQGKALNAAASRNVKVLVVGNPANTNAYIAMKSAPDLPAKNFTAMLRLDHNRAASQLAAKAGFKVGDIRKLTVWGNHSPTMYADYRFATVNGESVKAKINDQAWNKDVFLPTVGKRGAAIIAARGLSSAASAANAAIDHMRDWALGSGGEWVTMGVPSNGEYGIPAGIVFGFPVTTENGEYKIVEGLEIDAFSQECIDKTLAELQGEQDGVKHLL

SEQ ID NO: 20 - protein, 324 aa, Halomonas elongata
  HelMDH
MKDPVRIAITGAAGQISYSLAFRIASGDMLGKDQPVILQLLEIPPAMDALNGVVMELNDCAFPLVQDIVASDDPNVAFKDADFALLVGARPRGPGMERKDLLEANAAIFSVQGKALNDHASRDVRVLVVGNPANTNALIASCNAPDLDAGQFTAMMRLDHNRAKTQLAQKVGKHVTDVESMIVWGNHSATQYPDLAHCKVDGKPALELVDQAWYENDFIPTVQQRGAAIIKARGASSAASAASAAIDHMRDWALGSDGIVSMGIPADGSYGIEPGIMYSYPVVCKNGQYEIVQDLEIGDFSRERMNATEQELREERDAVEHLLG

SEQ ID NO: 21 - protein, 311 aa, Shewanella putrefaciens
  SpuMDH
MKVAVLGAAGGIGQALALLLKTQLPAGSKLSLYDIAPVTPGVAVDLSHIPTAVEIKGFAGEDPTPALVGADVVLISAGVARKPGMDRSDLFNINAGIVRNLIEKVAATCPKALVGIITNPVNTTVAIAAEVMKKAGVYDKNRLFGVTTLDVIRSETFIAELKGLNVADVKINVIGGHSGVTILPLLSQVEGVTFSDEEVASLTKRIQNAGTEVVEAKAGGGSATLSMGQAACRFGMSLVRGLQGEANVVECAYVDGGSEHAEFFAQPVLLGKNGIEKVLPYGEVSAFEANARDSMLDTLKGDIKLGVDFVK

SEQ ID NO: 22 - protein, 569 aa, Schizosaccharomyces pombe
  SpoPDC2
MTKDAESTMTVGTYLAQRLVEIGIKNHFVVPGDYNLRLLDFLEYYPGLSEIGCCNELNCAFAAEGYARSNGIACAVVTYSVGALTAFDGIGGAYAENLPVILVSGSPNTNDLSSGHLLHHTLGTHDFEYQMEIAKKLTCAAVAIKRAEDAPVMIDHAIRQAILQHKPVYIEIPTNMANQPCPVPGPISAVISPEISDKESLEKATDIAAELISKKEKPILLAGPKLRAAGAESAFVKLAEALNCAAFIMPAAKGFYSEEHKNYAGVYWGEVSSSETTKAVYESSDLVIGAGVLFNDYSTVGWRAAPNPNILLNSDYTSVSIPGYVFSRVYMAEFLELLAKKVSKKPATLEAYNKARPQTVVPKAAEPKAALNRVEVMRQIQGLVDSNTTLYAETGDSWFNGLQMKLPAGAKFEVEMQWGHIGWSVPSAMGYAVAAPERRTIVMVGDGSFQLTGQEISQMIRHKLPVLIFLLNNRGYTIEIQIHDGPYNRIQNWDFAAFCESLNGETGKAKGLHAKTGEELTSAIKVALQNKEGPTLIECAIDTDDCTQELVDWGKAVRSANARPPTADN

SEQ ID NO: 23 - protein, 565 aa, Schizosaccharomyces pombe
  Spomae2
MPAGTKEQIECPLKGVTLLNSPRYNKDTAFTPEERQKFEISSRLPPIVETLQQQVDRCYDQYKAIGDEPLQKNLYLSQLSVTNQTLFYALISQHLIEMIPIIYTPTEGDAIKQFSDIYRYPEGCYLDIDHNDLSYIKQQLSEFGKSDSVEYIIITDSEGILGIGDQGVGGVLISVAKGHLMTLCAGLDPNRFLPIVLDVGTNNETHRKNHQYMGLRKDRVRGEQYDSFLDNVIKAIREVFPEAFIHFEDFGLANAKRILDHYRPDIACFNDDIQGTGAVALAAIIGALHVTKSPLTEQRIMIFGAGTAGVGIANQIVAGMVTDGLSLDKARGNLFMIDRCGLLLERHAKIATDGQKPFLKKDSDFKEVPSGDINLESAIALVKPTILLGCSGQPGKFTEKAIREMSKHVERPIIFPISNPTTLMEAKPDQIDKWSDGKALIATGSPLPPLNRNGKKYVISQCNNALLYPALGVACVLSRCKLLSDGMLKAASDALATVPRSLFAADEALLPDLNNAREISRHIVFAVLKQAVSEGMSTVDLPKDDAKLKEWIIEREWNPEYKPFV

SEQ ID NO: 24 - DNA, 21 bp, Artificial Sequence
  Synthesized
ctctccagctccatccataag

SEQ ID NO: 25 - DNA, 45 bp, Artificial Sequence
  Synthesized
gacacaacttcctaccaaaaagcctttctgcccatgttttctgtc

SEQ ID NO: 26 - DNA, 23 bp, Artificial Sequence
  Synthesized
gctttttggtaggaagttgtgtc

SEQ ID NO: 27 - DNA, 43 bp, Artificial Sequence
  Synthesized
agtgggatttgtagctaagctgtatccatttcagccgtttgtg

SEQ ID NO: 28 - DNA, 45 bp, Artificial Sequence
  Synthesized
aagtttcgtcaatatcacaagctgacagaaaacatgggcagaaag

SEQ ID NO: 29 - DNA, 24 bp, Artificial Sequence
  Synthesized
gttccttagaaaaagcaactttgg

SEQ ID NO: 30 - DNA, 20 bp, Artificial Sequence
  Synthesized
cataagcttgccaccacttc

SEQ ID NO: 31 - DNA, 24 bp, Artificial Sequence
  Synthesized
gaaaaagcaactttggtattctgc

SEQ ID NO: 32 - DNA, 21 bp, Artificial Sequence
  Synthesized
agcttagctacaaatcccact

SEQ ID NO: 33 - DNA, 23 bp, Artificial Sequence
  Synthesized
agcttgtgatattgacgaaactt

SEQ ID NO: 34 - DNA, 5400 bp, Artificial Sequence
  Synthesized
tggggagcctggggactttccacaccctaactgacacacattccacaggacattgattattgactaggtttaaaccacgtgggtgaccaaattgtcaagcgtgagcttgccactggtgtccccattgtctaccacttggacaaggacggcaagtacgtctccaaggagctcattgacaactagatttcctactagattttagtcgcctattttaacgacatatacactgtttttctacactaactcatttctatgatgttgtataatgcaatttctttttttgaaatcaaatcaaactacaaggtagacgaaataatagagtaattatgagggagtaacaagggagtaacggggtggtggaagaagtgagtgagttggtagtgcaaggagagagaatcgtaccaatacattaggaggaagaaaaagtatcgatttagtagaaagaaatagcattatcgtactgtgtgaagagtttacagtcttgctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattttgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagatttctctttagttctttgcaagaaggtagagataaagacactttttcaaatatgagcgaaaaacaggttgtagggatccttggaggtggtcaattgggccgaatgatggtagaggcagcccatcgcttaaacatcaaatgcatcatcttggatgcagcaaattctcctgccaaacaaattgatggaggacgtgagcacattgatgcatcatttactgaccccgatgcaattgttgaactgtctaagaagtgcacgttattaacaactgaaattgagcatattaacactgatgccttggcagccgttacgaaatctgttgctgttgaaccctctcctgcaactctgcgatgcattcaagacaaatatcttcaaaaacagcatttacaggtttttaagattgcacttcctgaattttgcgatgcacctgaccaggaaagtgttgaaaaagcaggccaagagtttggttatccttttgtactgaaaagtaaaacattggcttacgacggtcgtggaaattacgttgttcatcaaccatctgagattcctactgccatcaaagcacttggtgatcgtccgctttatgttgaaaagttcgttcctttctccatggaaattgcagtgatggtagtacgcagtttagacggaaaagtttatgcttatcctacaactgagaccattcaaaaggataatgtttgtcatttagtatatgcccctgctcgtcttcccttctcaattcaacagcgtgctcaaacccttgccatggatgcggtgcgcacttttgaaggcgctggtatatatggtgtagagatgtttgttttgcaagatggtgagaccattttactcaacgaaattgctcctcggcctcacaattcaggtcactacaccattgaagcttgcccaacttctcagtttgaagctcacttacgggccatatgtggtcttcctttcagcgaaatcaacacccaactctcgacttccacaactcatgcgttgatggtaaatattttaggtactgatgatcctgattatgtttcaaagatcgctaaacgttctctgtccattcccggtgcaactttgcatctttatggtaaagctgaatctagaaagggtcgcaagatgggacacgttaccatcatttctgattcacctcaagaatgtgaacgtaggtatcagatgcttcttgacgtcaaagatcctgtcgaatcacctgttgttggtattatcatgggttcggattctgatttaagcaagatgaaagatgctgccgtcattttagatgaattcaaggtgccttacgaacttactattgtttcagctcaccgcacaccagatcgcatggttacttatgctcgtaccgcagcttcaagagggttgcgtgtgattattgctggtgctggtggtgccgctcatttgcctggtatggttgctgcaatgacacctcttccagtaatcggcgttcctgtaaaaggaagcactcttgacggagttgactctcttcactctattgttcagatgcctcgaggtgtccctgtcgccactgttgctatcaataatagccaaaatgccggtattttagcctgtcgtatacttgctacatttcaaccctcccttttggctgctatggagagctttatggacaatatgaaagagattgttttagaaaaggctgataaattagaaaaagttggttggaaaaattattctgcatagatggatgctagagtatttcaaagctattcagctagagctgaggggatgaaaaatcccattgccaaggaattgttggctttgatggaagaaaagcaaagcaacttgtcagtcgcggtcgatttgacgaagaaatccgaaatcttagaattggtagataaaattggaccctatgtctgtgttatcaagacacatattgacgttgtcgaggatttcgaccaggatatggtagaaaaactggtggccttaggtaaaaagcatcgttttcttatctttgaggatcgcaaattcgcagacattggaaataccgtcaagctacaatatgcatctggtgtgtacaaaattgcttcttgggctcatatcacaaattgccatacagtgccaggcgagggtattatacaaggcctcaaagaagttggtttacctttgggacgtggtctcttgcttttggctgaaatgtcttccaaaggctctttggctactggttcctacacagagaaaaccttagaatggtttgagaagcataccgatttttgctttggctttatagctggtcgtcgatttcctaaccttcaaagcgactacataactatgtcccctggtatcggcttggatgttaaaggagacgggctgggacagcaatatcgtactcctgaagaagtgattgtaaactgcggtagcgatatcatcattgttggtcgtggagtctatggagctggtcgtaatcctgttgtcgaagccaagagatatagagaagctggttggaaggcatatcagcaaagactttctcagcattaaaaaaagactaatgtaaaatttttttggttggttattgaaaaagtcgatgccttgtttgcgtttgttttcctaggcgttttatgtcagaaggcatttagaattagtatacaagtactctttggtaaaattttatgtagcgactaaaatattaactattatagataaacaccttgggaataaaaagtaatttgctatagtaatttattaaacatgctcctacaacattacctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattttgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagatttctctttagttctttgcaagaaggtagagataaagacactttttcaaacatgtcgattacaaggatgatgatgataagtaagaattcgagctcggtacccggggatcctctagagtcgacctgcaggcatgcaagcttaaataggaaagtttcttcaacaggattacagtgtagctacctacatgctgaaaaatatagcctttaaatcatttttatattataactctgtataatagagataagtccattttttaaaaatgttttccccaaaccataaaaccctatacaagttgttctagtaacaatacatgagaaagatgtctatgtagctgaaaataaaatgacgtcacaagacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtaatcgtttgtgaagagtttacagtcttgcagcaagtttctcaatgctttagagtgcctatgtctacgatggatgctacgtcttcctctgaatagtctattaaaaatcaacaatacctttttgttacaaggtgttgagtgtcattaaaagacaaagtgaaatacaaggggattattaagaaattatagtgcggcggagcaaagtagactagtagtccattttctatgcacagtagaaaacaaaaagtattcaacaaaaaaaaaaaaaaacagaatacattgcaaggattagaaagcaaacaacccttctttacaaggatgtaatgaagcatatttttattatcaaaaacattgatgatgaaattggtatgcttttggatactactattgctttttactctacgacaatgacatttcacattcaattggtttaaaccacgtggattactggcttaactatgcggcatcagagcagattgtactgagagtgcaccatatgcggtgtgaaatac

SEQ ID NO: 35 - DNA, 8849 bp, Artificial Sequence
  Synthesized
gaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcgatcccggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatccgggacctgaaataaaagacaaaaagactaaacttaccagttaactttctggtttttcagttcctcgaggagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacaggacattgattattgactaggtttaaaccacgtgggtgaccaaattgtcaagcgtgagcttgccactggtgtccccattgtctaccacttggacaaggacggcaagtacgtctccaaggagctcattgacaactagatttcctactagattttagtcgcctattttaacgacatatacactgtttttctacactaactcatttctatgatgttgtataatgcaatttctttttttgaaatcaaatcaaactacaaggtagacgaaataatagagtaattatgagggagtaacaagggagtaacggggtggtggaagaagtgagtgagttggtagtgcaaggagagagaatcgtaccaatacattaggaggaagaaaaagtatcgatttagtagaaagaaatagcattatcgtactgtgtgaagagtttacagtcttgctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattttgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagatttctctttagttctttgcaagaaggtagagataaagacactttttcaaatatgagcgaaaaacaggttgtagggatccttggaggtggtcaattgggccgaatgatggtagaggcagcccatcgcttaaacatcaaatgcatcatcttggatgcagcaaattctcctgccaaacaaattgatggaggacgtgagcacattgatgcatcatttactgaccccgatgcaattgttgaactgtctaagaagtgcacgttattaacaactgaaattgagcatattaacactgatgccttggcagccgttacgaaatctgttgctgttgaaccctctcctgcaactctgcgatgcattcaagacaaatatcttcaaaaacagcatttacaggtttttaagattgcacttcctgaattttgcgatgcacctgaccaggaaagtgttgaaaaagcaggccaagagtttggttatccttttgtactgaaaagtaaaacattggcttacgacggtcgtggaaattacgttgttcatcaaccatctgagattcctactgccatcaaagcacttggtgatcgtccgctttatgttgaaaagttcgttcctttctccatggaaattgcagtgatggtagtacgcagtttagacggaaaagtttatgcttatcctacaactgagaccattcaaaaggataatgtttgtcatttagtatatgcccctgctcgtcttcccttctcaattcaacagcgtgctcaaacccttgccatggatgcggtgcgcacttttgaaggcgctggtatatatggtgtagagatgtttgttttgcaagatggtgagaccattttactcaacgaaattgctcctcggcctcacaattcaggtcactacaccattgaagcttgcccaacttctcagtttgaagctcacttacgggccatatgtggtcttcctttcagcgaaatcaacacccaactctcgacttccacaactcatgcgttgatggtaaatattttaggtactgatgatcctgattatgtttcaaagatcgctaaacgttctctgtccattcccggtgcaactttgcatctttatggtaaagctgaatctagaaagggtcgcaagatgggacacgttaccatcatttctgattcacctcaagaatgtgaacgtaggtatcagatgcttcttgacgtcaaagatcctgtcgaatcacctgttgttggtattatcatgggttcggattctgatttaagcaagatgaaagatgctgccgtcattttagatgaattcaaggtgccttacgaacttactattgtttcagctcaccgcacaccagatcgcatggttacttatgctcgtaccgcagcttcaagagggttgcgtgtgattattgctggtgctggtggtgccgctcatttgcctggtatggttgctgcaatgacacctcttccagtaatcggcgttcctgtaaaaggaagcactcttgacggagttgactctcttcactctattgttcagatgcctcgaggtgtccctgtcgccactgttgctatcaataatagccaaaatgccggtattttagcctgtcgtatacttgctacatttcaaccctcccttttggctgctatggagagctttatggacaatatgaaagagattgttttagaaaaggctgataaattagaaaaagttggttggaaaaattattctgcatagatggatgctagagtatttcaaagctattcagctagagctgaggggatgaaaaatcccattgccaaggaattgttggctttgatggaagaaaagcaaagcaacttgtcagtcgcggtcgatttgacgaagaaatccgaaatcttagaattggtagataaaattggaccctatgtctgtgttatcaagacacatattgacgttgtcgaggatttcgaccaggatatggtagaaaaactggtggccttaggtaaaaagcatcgttttcttatctttgaggatcgcaaattcgcagacattggaaataccgtcaagctacaatatgcatctggtgtgtacaaaattgcttcttgggctcatatcacaaattgccatacagtgccaggcgagggtattatacaaggcctcaaagaagttggtttacctttgggacgtggtctcttgcttttggctgaaatgtcttccaaaggctctttggctactggttcctacacagagaaaaccttagaatggtttgagaagcataccgatttttgctttggctttatagctggtcgtcgatttcctaaccttcaaagcgactacataactatgtcccctggtatcggcttggatgttaaaggagacgggctgggacagcaatatcgtactcctgaagaagtgattgtaaactgcggtagcgatatcatcattgttggtcgtggagtctatggagctggtcgtaatcctgttgtcgaagccaagagatatagagaagctggttggaaggcatatcagcaaagactttctcagcattaaaaaaagactaatgtaaaatttttttggttggttattgaaaaagtcgatgccttgtttgcgtttgttttcctaggcgttttatgtcagaaggcatttagaattagtatacaagtactctttggtaaaattttatgtagcgactaaaatattaactattatagataaacaccttgggaataaaaagtaatttgctatagtaatttattaaacatgctcctacaacattacctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattttgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagatttctctttagttctttgcaagaaggtagagataaagacactttttcaaacatgtcgattacaaggatgatgatgataagtaagaattcgagctcggtacccggggatcctctagagtcgacctgcaggcatgcaagcttaaataggaaagtttcttcaacaggattacagtgtagctacctacatgctgaaaaatatagcctttaaatcatttttatattataactctgtataatagagataagtccattttttaaaaatgttttccccaaaccataaaaccctatacaagttgttctagtaacaatacatgagaaagatgtctatgtagctgaaaataaaatgacgtcacaagacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtaatcgtttgtgaagagtttacagtcttgcagcaagtttctcaatgctttagagtgcctatgtctacgatggatgctacgtcttcctctgaatagtctattaaaaatcaacaatacctttttgttacaaggtgttgagtgtcattaaaagacaaagtgaaatacaaggggattattaagaaattatagtgcggcggagcaaagtagactagtagtccattttctatgcacagtagaaaacaaaaagtattcaacaaaaaaaaaaaaaaacagaatacattgcaaggattagaaagcaaacaacccttctttacaaggatgtaatgaagcatatttttattatcaaaaacattgatgatgaaattggtatgcttttggatactactattgctttttactctacgacaatgacatttcacattcaattggtttaaaccacgtggattactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 36 - DNA, 31 bp, Artificial Sequence
  Synthesized
gacactttttcaaacatggctgctccccgcc

SEQ ID NO: 37 - DNA, 36 bp, Artificial Sequence
  Synthesized
atcatccttgtaatcggccttgacgatcttgcagac

SEQ ID NO: 38 - DNA, 45 bp, Artificial Sequence
  Synthesized
gacactttttcaaacatgaaaaagagaaaaatcaatcgcctactg

SEQ ID NO: 39 - DNA, 40 bp, Artificial Sequence
  Synthesized
atcatccttgtaatcctccatttcaatgagcagatcacca

SEQ ID NO: 40 - DNA, 44 bp, Artificial Sequence
  Synthesized
gacactttttcaaacatgagtgacggtaatgaacataagatcaa

SEQ ID NO: 41 - DNA, 45 bp, Artificial Sequence
  Synthesized
atcatccttgtaatctttaacaacacttgcaatcaaatcgttagc

SEQ ID NO: 42 - DNA, 40 bp, Artificial Sequence
  Synthesized
gacactttttcaaacatgtcatctcaactagctggattgc

SEQ ID NO: 43 - DNA, 37 bp, Artificial Sequence
  Synthesized
atcatccttgtaatcttccttagctggagcttcttcc

SEQ ID NO: 44 - DNA, 36 bp, Artificial Sequence
  Synthesized
gacactttttcaaacatgtccaaactggcaggactg

SEQ ID NO: 45 - DNA, 39 bp, Artificial Sequence
  Synthesized
atcatccttgtaatcttcttttggtgggacagattcctc

SEQ ID NO: 46 - DNA, 43 bp, Artificial Sequence
  Synthesized
gacactttttcaaacatgacatcaattgcttcatcaaacactc

SEQ ID NO: 47 - DNA, 47 bp, Artificial Sequence
  Synthesized
atcatccttgtaatcatgtttcaaaatactggtgatcaaatcatttg

SEQ ID NO: 48 - DNA, 37 bp, Artificial Sequence
  Synthesized
gacactttttcaaacatgtcgcaaagaaaattcgccg

SEQ ID NO: 49 - DNA, 40 bp, Artificial Sequence
  Synthesized
atcatccttgtaatctgccttagtttcaacaggaacttgg

SEQ ID NO: 50 - DNA, 54 bp, Artificial Sequence
  Synthesized
cactttttcaaacatgatttttataaattcatcatcaatttctacaaaatctcc

SEQ ID NO: 51 - DNA, 47 bp, Artificial Sequence
  Synthesized
atcatccttgtaatcggctttcaaaatactggtgattaaatcattag

SEQ ID NO: 52 - DNA, 40 bp, Artificial Sequence
  Synthesized
cactttttcaaacatgtctgtaccagtccaaaaagctaac

SEQ ID NO: 53 - DNA, 51 bp, Artificial Sequence
  Synthesized
atcatccttgtaatcatgttgaataatgttagtaatcaaatcattagcatc

SEQ ID NO: 54 - DNA, 41 bp, Artificial Sequence
  Synthesized
cactttttcaaacatgtccataaacaaaaaactaaacggcc

SEQ ID NO: 55 - DNA, 42 bp, Artificial Sequence
  Synthesized
atcatccttgtaatcagatggaggcgtagattcttctaaaag

SEQ ID NO: 56 - DNA, 44 bp, Artificial Sequence
  Synthesized
cactttttcaaacatgtcgaacaaaaaattcaatggtatcagag

SEQ ID NO: 57 - DNA, 41 bp, Artificial Sequence
  Synthesized
atcatccttgtaatctgcttcttccggaatttcagtttctt

SEQ ID NO: 58 - DNA, 36 bp, Artificial Sequence
  Synthesized
cactttttcaaacatgtcgcaaaagaaattcgctgg

SEQ ID NO: 59 - DNA, 41 bp, Artificial Sequence
  Synthesized
atcatccttgtaatctgctttagtttcagctggaacttgat

SEQ ID NO: 60 - DNA, 41 bp, Artificial Sequence
  Synthesized
cactttttcaaacatgacttctaaatatgatgcgttgttgc

SEQ ID NO: 61 - DNA, 38 bp, Artificial Sequence
  Synthesized
atcatccttgtaatcctcgtgttcaagaacagcacaca

SEQ ID NO: 62 - DNA, 45 bp, Artificial Sequence
  Synthesized
cactttttcaaacatgacaaataagaaattctctggtctaagaga

SEQ ID NO: 63 - DNA, 43 bp, Artificial Sequence
  Synthesized
atcatccttgtaatcttttggtggttctggatcttctaatgtg

SEQ ID NO: 64 - DNA, 37 bp, Artificial Sequence
  Synthesized
cactttttcaaacatgtccaagaacaagaggtttgct

SEQ ID NO: 65 - DNA, 36 bp, Artificial Sequence
  Synthesized
atcatccttgtaatcgttttcagctgggacagcctc

SEQ ID NO: 66 - DNA, 39 bp, Artificial Sequence
  Synthesized
cactttttcaaacatgagtaccaagaagttctctggtct

SEQ ID NO: 67 - DNA, 35 bp, Artificial Sequence
  Synthesized
atcatccttgtaatcattctcctttggtgggacgg

SEQ ID NO: 68 - DNA, 35 bp, Artificial Sequence
  Synthesized
actttttcaaacatgaaactcggttttgttggtgc

SEQ ID NO: 69 - DNA, 45 bp, Artificial Sequence
  Synthesized
atcatcatcatccttgtaatcatatccgagttcctcaagcctctc

SEQ ID NO: 70 - DNA, 32 bp, Artificial Sequence
  Synthesized
actttttcaaacatgaaagcacctgttcgcgt

SEQ ID NO: 71 - DNA, 40 bp, Artificial Sequence
  Synthesized
atcatcatcatccttgtaatcgggcagaaggtctgccacg

SEQ ID NO: 72 - DNA, 32 bp, Artificial Sequence
  Synthesized
actttttcaaacatgagcaagaagcccgttcg

SEQ ID NO: 73 - DNA, 39 bp, Artificial Sequence
  Synthesized
atcatcatcatccttgtaatccagcaggtgcttgacgcc

SEQ ID NO: 74 - DNA, 35 bp, Artificial Sequence
  Synthesized
actttttcaaacatgaaagaccccgtccgtatagc

SEQ ID NO: 75 - DNA, 40 bp, Artificial Sequence
  Synthesized
atcatcatcatccttgtaatcgccaagcagatgctcgacg

SEQ ID NO: 76 - DNA, 35 bp, Artificial Sequence
  Synthesized
actttttcaaacatgaaagttgctgtacttggtgc

SEQ ID NO: 77 - DNA, 46 bp, Artificial Sequence
  Synthesized
atcatcatcatccttgtaatctttaacgaaatcaacgcctagtttg

SEQ ID NO: 78 - DNA, 20 bp, Artificial Sequence
  Synthesized
aggctttgatagccactggt

SEQ ID NO: 79 - DNA, 40 bp, Artificial Sequence
  Synthesized
tgggatttgtagctaagctttaaaataaaaggctttatac

SEQ ID NO: 80 - DNA, 39 bp, Artificial Sequence
  Synthesized
ttcgtcaatatcacaagcttaggtcgactgggctaatcg

SEQ ID NO: 81 - DNA, 40 bp, Artificial Sequence
  Synthesized
taaaataaaaggctttatacaggcattttcaaacttcaag

SEQ ID NO: 82 - DNA, 40 bp, Artificial Sequence
  Synthesized
cttgaagtttgaaaatgcctgtataaagccttttatttta

SEQ ID NO: 83 - DNA, 27 bp, Artificial Sequence
  Synthesized
ttcattcaatacataacggtttacggt

SEQ ID NO: 84 - DNA, 20 bp, Artificial Sequence
  Synthesized
attcgtgaaatgagcaagca

SEQ ID NO: 85 - DNA, 25 bp, Artificial Sequence
  Synthesized
tgcgatttactatttgtttgtttca

SEQ ID NO: 86 - DNA, 24 bp, Artificial Sequence
  Synthesized
ctcaagtaatgggcaatcatgcca

SEQ ID NO: 87 - DNA, 40 bp, Artificial Sequence
  Synthesized
tgggatttgtagctaagcttagagtggtaaaaaattatac

SEQ ID NO: 88 - DNA, 40 bp, Artificial Sequence
  Synthesized
ttcgtcaatatcacaagcttaataaaatacacaaaactgt

SEQ ID NO: 89 - DNA, 40 bp, Artificial Sequence
  Synthesized
agagtggtaaaaaattatacagccatgtgggttattttaa

SEQ ID NO: 90 - DNA, 40 bp, Artificial Sequence
  Synthesized
ttaaaataacccacatggctgtataattttttaccactct

SEQ ID NO: 91 - DNA, 28 bp, Artificial Sequence
  Synthesized
gagcaaccgaatatcaaggaaatacaca

SEQ ID NO: 92 - DNA, 23 bp, Artificial Sequence
  Synthesized
aggcgaattgatccttcctgcta

SEQ ID NO: 93 - DNA, 29 bp, Artificial Sequence
  Synthesized
tggtaagcctggtatgagttctatactat

SEQ ID NO: 94 - protein, 544 aa, Candida glabrata
  CglPCK
MPSKSSVSGNSVEERIRSELGLSKEVTLIRRNAPAAVLYQDALKEKKTVISSAGALIAYSGEKTGRSPKDKRIVEEETSRDNVWWGPVNKPCSERTWAINRERAADYLRTRETLYVVDAFAGWDPKYRIKVRVVCARAYHALFMTNMLIRPTEEELANFGEPDFTVWNAGQFPANARTQDMTSKTTIEINFKAMEMVILGTEYAGEMKKGIFTVMFYLMPVHHNVLTLHSSCNQGIKNGDVTLFFGLSGTGKTTLSADPHRLLIGDDEHCWSDEGVFNIEGGCYAKCINLSREKEPEIFDAIRFGSVLENVIYDSESHEVDYDDSSITENTRCAYPIDFIPSAKIPCLAPAHPKNIILLTCDASGVLPPVSKLTPEQVMYHFISGYTSKMAGTEQGVTEPEPTFSSCFGQPFLSLHPMRYATMLAEKMHEHSANAWLINTGWTGSSYVSGGKRCALKYTRAILDAIHDGSLAKAEFESLPIFNLQVPKAVEGVPSELLNPARNWSEGEAKYQSAVSKLAGLFVENFKTYQDKATSDVLAAGPQL

SEQ ID NO: 95 - protein, 539 aa, Citrobacter koseri
  CkoPCK
MRVNSLTPQDLKAYGINDVQDIVYNPSYDLLYQEELDPTLEGYERGVLTNLGAVAVDTGIFTGRSPKDKYIVRDDTTRDTLWWSDKGKGKNDNKPLSQETWQHLKGLVTQQLSGKRLFIVDAFCGANADTRLSVRFITEVAWQAHFVKNMFIRPADEELVDFKPDFIVMNGAKCTNPQWKEQGLNSENFVAFNLTERIQLIGGTWYGGEMKKGMFSVMNYLLPLKGIASMHCSANVGEKGDVAVFFGLSGTGKTTLSTDPKRRLIGDDEHGWDDDGVFNFEGGCYAKTIKLSKEAEPEIYNAIRRDALLENVTVREDGSIDFDDGSKTENTRVSYPIYHIDNIVKPVSKAGHATKVIFLTADAFGVLPPVSRLTADQTQYHFLSGFTAKLAGTERGVTEPTPTFSACFGAAFLSLHPTQYAEVLVKRMQASGAQAYLVNTGWNGTGKRISIKDTRAIIDAILNGSLDNAETFNLPMFDLAIPTELPGVETRILDPRNTYASPEQWQEKATALAKLFVENFEKYTDTPAGEALVSAGPKL

SEQ ID NO: 96 - protein, 539 aa, Cronobacter sakazakii
  CsaPCK
MRVTGITPQDLKAYGINDVQEVVYNPDYDTLYREELDPSLEGYERGVLTDLGAVAVDTGIFTGRSPKDKYIVRDDTTRDTLWWSDNGKGKNDNKPLTPETWQHLKGLVTQQLSGKRLFIIDAFCGANPDSRLSVRFITEVAWQAHFVKNMFIRPSEDELEGFEPDFIVMNGAKCTNPDWQEQGLNSENFVAFNLTERMQLIGGTWYGGEMKKGMFSIMNYLLPLKGIASMHCSANVGEKGDVAVFFGLSGTGKTTLSTDPKRRLIGDDEHGWDDDGVFNFEGGCYAKTIRLSEEAEPDIYHAIRRDALLENVTVRDDGSIDFDDASKTENTRVSYPIYHIDNIVKPVSKAGHATKVIFLTADAFGVLPPVSRLTASQTQYHFLSGFTAKLAGTERGVTEPTPTFSACFGAAFLMLHPTQYSEVLVKRMQAAGAQAYLVNTGWNGTGKRISIKDTRAIIDAILNGSLDDAETFTLPLFNLAIPTSLPGVDERILDPRNTYASPEQWQEKAQQLAQLFISNFEKYTDTPAGAALVSAGPQR

SEQ ID NO: 97 - protein, 553 aa, Debaryomyces hansenii
  DhaPCK
MTPPTAVESSINFGGHPTIKSTAEPVVKQLSLADDTVIRHNAPPPTLYEDGLLEKGTVISSTGALMAYSGKKTGRSPKDKRIVDEETSTQNIWWGPVNKQVDELTWKISRSRALDYLRTREKLFVVDAYAGWDPRYRLKIRVICARAYHALFMTNMLIRPTEEELKNFGEPDFTIYNAGQFPANVHTKGMTSSTSVEINFKDMEMVILGTEYAGEMKKGIFTVMFYLMPIKHKILTLHSSANQGEQKGDVTLFFGLSGTGKTTLSADPNRKLIGDDEHCWSDNGVFNIEGGCYAKCLDLSAEKEPEIFNSIKFGAILENVVYDQNSKVVDYADSTITENTRCAYPIDFIPSAKIPCLADEHPTNIVLLTCDASGVLPPVSKLTNAQVMYHFISGYTSKMAGTEEGVTEPQATFSACFGQPFLVLHPMKYAQQLSDKISKHNANAWLLNTGWVGASAAQGGNRCSLKYTRAILDAIHSGELSKVEFENFPTFNLNVPKSCPNVPSEILNPTKAWTEGDASFQKEIKSLAGKFAENFTKYADQATTEVKAAGPDA

SEQ ID NO: 98 - protein, 540 aa, Escherichia fergusonii
  EfePCK
MRVNKSLTPQDLMAYGINDVQDIVYNPSYDLLFQEELDPNLKGYERGVLTNLGAVAVDTGVFTGRSPKDKYIVRDDTTRDTFWWADKGKGKNDNKPLSPETWQHLKGLVTNQLSGKRLFVVDAFCGANADTRLSVRFITEVAWQAHFVKNMFIRPTDEELVDFEPDFIVMNGAKCTNPQWKEQGLNSENFVAFNLTERMQLIGGTWYGGEMKKGMFSMMNYLLPLKGIASMHCSANVGEKGDVAVFFGLSGTGKTTLSTDPKRRLIGDDEHGWDDDGVFNFEGGCYAKTIKLSKEAEPEIYNAIRRDALLENVTVREDGTIDFDDGSKTENTRVSYPIYHIDNIVKPVSKAGHATKVIFLTADAFGVLPPVSRLTADQTQYHFLSGFTAKLAGTERGITEPTPTFSACFGAAFLSLHPTQYAEVLVKRMQAAGAQAYLVNTGWNGTGKRISIKDTRAIIDAILNGSLDNAETFTLPMFNLAIPTELPGVDTEILDPRNTYASPEQWQEKAETLAKLFIDNFDKYTDTPAGAALVEAGPKM

SEQ ID NO: 99 - protein, 539 aa, Edwardsiella tarda
  EtaPCK
MHATGLTAADLASYGISDVTEIVYNPDYDLLFKEETAPGLQGYERGTVTTLGAVAVDTGIFTGRSPKDKYLVRDDTTRDTVWWSDQGKGKNDNHPLSPEIWQHLKGLVTRQLSNKRLFVVDAYCGANPDSRLKVRFITEVAWQAHFVKNMFIRPTEDELAGFEPDFIVMNGAKCTNPQWQEQGLNSENFVAFNLTERIQLIGGTWYGGEMKKGMFSIMNYLLPLKGIASMHCSANVGEKGDVAVFFGLSGTGKTTLSTDPKRRLIGDDEHGWDDDGVFNFEGGCYAKTIKLSAEAEPDIYHAITRDALLENVTVRADGSVDFDDGSKTENTRVSYPIYHIKNIVKPVSKAGHAKKVIFLTADAFGVLPPVSRLTADQTQYHFLSGFTAKLAGTERGVTEPTPTFSACFGAAFLTLHPTQYAEVLVKRMQAAGAQAYLVNTGWNGSGKRISIKDTRAIIDAILNGDIDKEETFTLPIFNLAVPTALPGVNPAILDPRDTYASPEMWQEKADDLAQRFITNFDKYTDTPAGAALVHAGPQR

SEQ ID NO: 100 - protein, 543 aa, Kluyveromyces lactis
  KlaPCK
MSPTKTQNSAEENIRAELGLSAEVVTIRRNAPAALLYEDALKERDTAISNAGALIAYSGDKTGRSPRDKRIVEEETSKDNVWWGPVNKPCSERTWEINRERAADYLRTRDHIYIVDAYAGWDPRYRIKVRVVCARAYHALFMTNMLIRPSKEELENFGEPDFTVWNAGQFPANTHTSGMTSKTTVEINFKQMEMVILGTEYAGEMKKGIFTVMFYLMPVNHNVLTLHSSANQGIQDNDVTLFFGLSGTGKTTLSADPHRLLIGDDEHCWSDHGVFNIEGGCYAKCLGLSAEKEPEIFNAIKFGSVLENIIYDPNTREVDYEDSTITENTRCAYPIEYIPSAKIPCLADHHPKNIVLLTCDASGVLPPVSKLTPDQVMYHFISGYTSKMAGTEQGVTEPEATFSSCFGQPFLSLHPMKYATMLAEKMAEHNANAWLINTGWTGSSYVAGGKRCPLKYTRAILDAIHDGSLAKEEYEVLPIFNLQIPKAVGDKVPASLLNPSKNWAEGEAKYTSNVKSLANLFVENFKTYQDKATEQVLAAGPQL

SEQ ID NO: 101 - protein, 542 aa, Lodderomyces elongisporus
  LelPCK
MAPPAVESTINFEGHPSIKTTVDPLVKKLNLKGDTVIRHNAPPPTLYEDGLLEKGTTISSTGALMAYSGEKTGRSPKDKRIVDEETSSQHIWWGPVNKQVDELTWKISRSRALDYLRTREKLFVVDAFAGWDPKYRIKVRIICARAYHALFMTNMLIRPTKEELENFGEPDFTIYNAGQFPANVHTKGMTSSTSVEINFKDMEMVILGTEYAGEMKKGIFTVMFYLMPIKNKVLTLHSSANEGESGDVTLFFGLSGTGKTTLSADPNRKLIGDDEHCWSDHGVFNIEGGCYAKCLDLSAEKEPEIFNSIRFGSVLENVVYDPATKVVDYEDSSITENTRCSYPIEYIPSLKIPCVGGHPKNIVLLTCDASGVLPPVSKLTPAQVMYHFISGYTSKMAGTEMGITEPTPAFSACFGQPFLVLHPMKYAQQLAEKIEQHKANAYLLNTGWSGVGAAKGGKRCPLKYTRAILDAIHSGELDSADTQVFSTFNLHVPKEIKGVPLEVLHPEVDKKEVAALAAKFAENFVKYADQATAEVKAAGPDV

SEQ ID NO: 102 - protein, 542 aa, Pectobacterium carotovorum; identical to SEQ ID NO: 101
  PcaPCK

SEQ ID NO: 103 - protein, 543 aa, Photobacterium leiognathi
  PlePCK
MSTMCVDNKVATNMDLSQYGILNVTDVIRNPSYEMLFEEETKAELKGFEKGIVTELGAVAVDTGIFTGRSPQDKFIVKDDTTKDTLWWSDQGKNDNKALSQGAWNDLKSLVTTQLSNKRLFVVDGYCGANPDTRLCIRVITEVAWQAHFVKNMFIRPTEAELDTFEPDFVIMNGSKCTNPKWQEHGMNSENFTVFNLSEKMQLIGGTWYGGEMKKGMFAMMNYFLPLKDIASMHCSANMGEAGDVAIFFGLSGTGKTTLSTDPKRALIGDDEHGWNDNGVFNFEGGCYAKTINLSKEAEPDIYNAIRRDALLENVTVRGDGSIDFNDNSKTENTRVSYPIYHIDNIVKPVSKGGHANKVIFLSADAFGVLPPVSKLTPEQTKYHFLSGFTAKLAGTERGITEPTPTFSACFGNAFLTLHPTQYAEVLVKRMEAAGAEAYLVNTGWNGTGKRISIQDTRGIIDAILDGSIDTAPTKQIPIFNLTVPTALPGVDPEILDPRDTYTDPLQWESKATDLAGRFIANFDKYTDTTEGKSLVKAGPQLD

SEQ ID NO: 104 - protein, 539 aa, Providencia rettgeri
  PrePCK
MSAKSITLKELEKYGIHDVTEVVYNPSYELLFTEETKPGLEGYERGTVTTLGAVAVDTGIFTGRSPKDKYIVRDDVTRDTVWWADQGKGKNDNKPMSQEVWADLKHLVTEQLSGKRLFIIDAFCGANADTRLKVRFITEVAWQAHFVKNMFIRPSDEELVGFEPDFIVMNGAKCTNPNWKAQGLNSENFVAFNLTERMQLIGGSWYGGEMKKGMFSMMNYLLPLKGIASMHCSANVGEKGDVAIFFGLSGTGKTTLSTDPKRKLIGDDEHGWDDDGVFNFEGGCYAKTINLSKEAEPDIYGAIKRDALLENVMVLADGSVDFNDGSKTENTRVSYPIYHIENIVKPVSKAGHATKVIFLTADAFGVLPPVSRLTPEQTQYHFLSGFTAKLAGTERGVTEPTPTFSACFGAAFLSLHPTQYAEVLVKRMEAAGAKAYLVNTGWNGTGKRISIKDTRAIIDAILSGDIEKADMIKLPVFDLEIPTALPGVDTNILDPRNTYADKAQWDEKAQDLAERFVNNFDKYTDTPAGAALVKAGPKL

SEQ ID NO: 105 - protein, 549 aa, Saccharomyces cerevisiae
  ScePCK
MSPSKMNATVGSTSEVEQKIRQELALSDEVTTIRRNAPAAVLYEDGLKENKTVISSSGALIAYSGVKTGRSPKDKRIVEEPTSKDEIWWGPVNKPCSERTWSINRERAADYLRTRDHIYIVDAFAGWDPKYRIKVRVVCARAYHALFMTNMLIRPTEEELAHFGEPDFTVWNAGQFPANLHTQDMSSKSTIEINFKAMEMIILGTEYAGEMKKGIFTVMFYLMPVHHNVLTLHSSANQGIQNGDVTLFFGLSGTGKTTLSADPHRLLIGDDEHCWSDHGVFNIEGGCYAKCINLSAEKEPEIFDAIKFGSVLENVIYDEKSHVVDYDDSSITENTRCAYPIDYIPSAKIPCLADSHPKNIILLTCDASGVLPPVSKLTPEQVMYHFISGYTSKMAGTEQGVTEPEPTFSSCFGQPFLALHPIRYATMLATKMSQHKANAYLINTGWTGSSYVSGGKRCPLKYTRAILDSIHDGSLANETYETLPIFNLQVPTKVNGVPAELLNPAKNWSQGESKYRGAVTNLANLFVQNFKIYQDRATPDVLAAGPQFE

SEQ ID NO: 106 - protein, 539 aa, Serratia odorifera
  SodPCK
MRAKGITHQDLAAYGIHDIGEIIHNPSYDLLFEEETDPSLTGYERGVVTKLGAVAVDTGIFTGRSPKDKYIVRDDTTRDTVWWADQGKGKNDNKPMSPEVWASLKTLVTEQLSGKRLFVVDTFCGANADSRLKVRFITEVAWQAHFVKNMFIRPSDDELADFEPDFVVMNGAKCTNPDWQQQGLNSENFVAFNLTERMQLIGGTWYGGEMKKGMFSIMNYLLPLKGIASMHCSANVGEQGDVAIFFGLSGTGKTTLSTDPKRQLIGDDEHGWDDDGVFNFEGGCYAKTIKLSEQAEPDIYRAIKRDALLENVTVLADGSVDFNDGSKTENTRVSYPIYHIQNIVKPVSKAGHATKVIFLTADAFGVLPPVSRLTADQTQYHFLSGFTAKLAGTERGVTEPTPTFSACFGAAFLSLHPTQYAEVLVKRMQAAGAQAYLVNTGWNGSGKRISIQDTRGIIDAILSGEIDNAETITLPIFDLAVPTALPGVNPAILDPRATYQSEAQWEDKAKDLAQRFITNFDKYTDTPAGEALIGAGPKL

SEQ ID NO: 107 - protein, 543 aa, Vibrio orientalis
  VorPCK
MTVMEHTKAATIDLTKHGLHNVKEVIRNPSYEMLFEEETRADLEGYEKGVVTELGAVAVDTGIFTGRSPKDKYIVKDATTEEHMWWTSDAVKNDNKPIDQAVWNDLKAQVTEQLSGKRVFVIDGYCGANPDTRLSIRVITEVAWQAHFVKNMFIRPSEEELTTFEPDFVVMNGAKCTNQKWEQHGLNSENFTVFNLTERMQLIGGTWYGGEMKKGMFAMMNYFLPLKDIASMHCSANKCKENGDVAIFFGLSGTGKTTLSTDPKRELIGDDEHGWDDDGVFNFEGGCYAKTIKLSKEAEPDIYNAIRRDALLENVTVRNDGSIDFDDGSKTENTRVSYPIEHIENIVKPVSRAGHANKVIFLSADAFGVLPPVSKLTPEQTKYHFLSGFTAKLAGTERGITEPTPTFSACFGAAFLTLHPTKYAEVLVKRMEAAGAEAYLVNTGWNGSGKRISIQDTRGIIDAILDGSIEEAETKHIPIFNLEVPTALHDVDPSILDPRETYTDPLQWESKAKDLASRFINNFDKYTDNDEGKSLVAAGPQLD

SEQ ID NO: 108 - protein, 558 aa, Vanderwaltozyma polyspora
  VpoPCK
MAPTKIEDYSLNERENIIKNLKSNSNVKLDNVMKCLYNKDITIKRNPPIAELYEDGLKEDSTTLSSTGALMAYSGVKTGRSPKDKRIVEEPSSKDNIWWGPVNKPCSELTWSINRERAIDYLNTRKILYVIDAYAGWDANYRIKIRVVCARPYHALFMNNMLIRPTTEELENFGEPDFTIWNAGSFYANSLTKDMTSKTSIEINFKSMEMVILGTEYAGEMKKGIFTVMFYLMPVYHNVLTLHSSANQGVNNGDVTLFFGLSGTGKTTLSADPHRELIGDDEHCWSDHGVFNIEGGCYAKCIGLTEEKEPEIFNAIKFGSVLENCVYDENTHVVDYDDSRITENTRCAYPIDFIESAKIPCLTDKHPTNLILLTCDASGVLPPVSKLTPEQVMYHFISGYTSKMAGTEQGVTEPEPTFSACFGQPFLALHPMKYAQMLADKMNHHKASAWLINTGWTGASYTAGGKRCPLKYTRAILDAIHNGSLANEEYETLPVFNLPIPKNVENVPSELLNPSKTWSDPDVTYSQAVKKLGSLFVQNFTIYQDQATDDVIAAGPAL

SEQ ID NO: 109 - protein, 543 aa, Vibrio tubiashii
  VtuPCK
MTVMEHTKAATIDLTKHGLQNVKEVIRNPSYEMLFEEETRADLEGYEKGVVTELGAVAVDTGIFTGRSPKDKYIVKDATTEEHMWWTSDAVKNDNKPIDQAVWNDLKQLVTNQLSGKRVFVIDGYCGANPDTRLSIRVITEVAWQAHFVKNMFIRPSEKELATFEPDFVVMNGAKCTNQKWEEHGLNSENFTVFNLTERMQLIGGTWYGGEMKKGMFAMMNYFLPLKDIASMHCSANKCKENGDVAVFFGLSGTGKTTLSTDPKRELIGDDEHGWDDDGIFNFEGGCYAKTIKLSKEAEPDIYNAIRRDALLENVTVRSDGSIDFDDGSKTENTRVSYPIEHIENIVKPVSKAGHANKVIFLSADAFGVLPPVSKLTPEQTKYHFLSGFTAKLAGTERGITEPTPTFSACFGAAFLTLHPTKYAEVLVKRMEAAGAEAYLVNTGWNGSGKRISIQDTRGIIDAILDGSIENAETKHIPIFNLEVPTALHDVDPAILDPRDTYTDPLQWESKAKDLAERFINNFDKYTDNDEGKSLVAAGPQLD

SEQ ID NO: 110 - protein, 539 aa, Yersinia bercovieri
  YbePCK
MSVKGITPQELAAYGIHNVSEIVYNPSYDLLFQEETKPTLEGYERGTLTTTGAIAVDTGIFTGRSPKDKYIVRDAITQDTVWWADQGKGKNDNKPLSQETWSHLKGLVTEQLSGKRLFVVDTFCGANADSRLQVRFVTEVAWQAHFVKNMFIRPTDEELADFEPDFIVMNGAKCTNPNWKEQGLNSENFVAFNLTERMQLIGGTWYGGEMKKGMFSMMNYLLPLKGIASMHCSANVGEKGDVAIFFGLSGTGKTTLSTDPKRKLIGDDEHGWDDDGVFNFEGGCYAKTIKLSEEAEPDIYHAIRRDALLENVVVLPDGTVDFNDSSKTENTRVSYPIYHIENIVKPVSKAGHATKVIFLTADAFGVLPPVSRLTANQTQYHFLSGFTAKLAGTERGVTEPTPTFSACFGAAFLSLHPTQYAEVLVKRMQAVGAQAYLVNTGWNGTGKRISIKDTRAIIDAILNGEIDKAETFTLPIFDLAVPMALPGVDPAILDPRDTYADIAQWQEKAEDLARRFTTNFDKYTDTPAGAALMSAGPKI

SEQ ID NO: 111 - protein, 556 aa, Yarrowia lipolytica
  YliPCK
MSPPVNQTQLHPKGVEPTPAAEEIEKELYDIAHIEYERVNIDHNPSVASLYEDALVQESGSAINSTGALVASSGKKTGRSPKDKRIVEEPGSVEHIWWGPVNKKLSEQSWLINRERAVDYLNTRKRIYVVDAYAGWDPKYRIKVRIVCARAYHALFMRNMLIRPTAEELKNFGKPDFTIYNAGAFPANRYTSGMTSNTSIDINFKSMEMLILGTEYAGEMKKGIFTVMFYLMPVKHQVLTLHSSANEGKDGDVTLFFGLSGTGKTTLSADPNRLLIGDDEHCWSDTGVFNIEGGCYAKCLGLSREKEPDIFDAIKFGSVLENVVFDPITREVDYDDATLTENTRCSYPIEYIPNAKLPCVTESHPTNIILLTCDARGVIPPISKLTNEQVMYHFISGYTSKMAGTEEGVTEPEATFSACFGQPFLVLHPMQYAKMLSDKMTQHNANAWLLNTGWTGQSYTNGGKRCPLKYTRSILDAIHSGELAKVEYENFDVFNLQVPKTAPNVPSEILNPARAWSGGKEQFTEEVTKLGKLFNENFEKYKDMAIPEVIAAGPKA

SEQ ID NO: 112 - protein, 539 aa, Yersinia rohdei
  YroPCK
MSVKGITPQELAAYGIHNVSEIVYNPSYDLLFQEETKPTLEGYERGTLTTTGAIAVDTGIFTGRSPKDKYIVRDDITRDTVWWADQGKGKNDNKPLSQETWAHLKGLVTQQLSGKRLFVVDTFCGANADTRLQVRFVTEVAWQAHFVKNMFIRPTDEELAHFEPDFIVMNGAKCTNPNWKEQGLNSENFVAFNLTERMQLIGGTWYGGEMKKGMFSMMNYLLPLKGIASMHCSANVGEKGDVAIFFGLSGTGKTTLSTDPKRKLIGDDEHGWDDDGVFNFEGGCYAKTIKLSEEAEPDIYHAIKRDALLENVVVLPDGTVDFNDGSKTENTRVSYPIYHIENIVKPVSKAGHATKVIFLTADAFGVLPPVSRLTASQTQYHFLSGFTAKLAGTERGVTEPTPTFSACFGAAFLSLHPTQYAEVLLKRMQAVGAQAYLVNTGWNGTGKRISIKDTRGIIDAILNGEIDKAETFTLPIFDLAVPMALPGVNPDILDPRDTYADVAQWQEKAEDLAKRFTTNFDKYTDTPAGAALVSAGPKV

SEQ ID NO: 113 - protein, 548 aa, Zygosaccharomyces rouxii
  ZroPCK
MSPSKVPVPITDTAEVEEQIRKELALGSEVVTIRHNAPAATLYEDGLKEKKTAVVSSGALAAYSGSKTGRSPKDKRIIEEETSKNEVWWGPVNKPASEKTWSINRERAADYLRTRDHLYIIDAYAGWDPRYRIKVRVICARAYHALFMTNMLIRPSPEELANFGEPDFTVWNAGQFPANRNTEGMTSKTTVEINFKAMEMIILGTEYAGEMKKGIFTVMFYLMPVHHNVLTLHSSANQGIERGDVTLFFGLSGTGKTTLSADPHRALIGDDEHCWSDHGVFNIEGGCYAKCIDLTAEKEPEIYNAIKFGSVLENVVFDASNREVDYTDNSITENTRCAYPIDYIPSAKIPCLADKHPSNIVLLTCDASGVLPPVSKLTPEQVMYHFISGYTSKMAGTEQGVTEPETTFSSCFGQPFLALHPMRYATMLAKKMHQHNANAWLVNTGWTGSSYTAGGKRCPLKYTRAILDSIHDGSLSKADYETLPIFNLHTPAKVNGVPDSLLNPAKSWVEGPTKYNSAVKTLATKFTENFKIYQDQATPDVLAAGPQF

SEQ ID NO: 114 - DNA, 41 bp, Artificial Sequence
  Synthesized
gacactttttcaaaatgccatctaaatctagtgtttctggg

SEQ ID NO: 115 - DNA, 38 bp, Artificial Sequence
  Synthesized
gaaatcaacttttgttccaattgtggaccagcagccaa

SEQ ID NO: 116 - DNA, 36 bp, Artificial Sequence
  Synthesized
gacactttttcaaaatgcgcgttaacagcttaaccc

SEQ ID NO: 117 - DNA, 32 bp, Artificial Sequence
  Synthesized
gaaatcaacttttgttccagcttcggcccggc

SEQ ID NO: 118 - DNA, 36 bp, Artificial Sequence
  Synthesized
gacactttttcaaaatgcgagttacaggcataaccc

SEQ ID NO: 119 - DNA, 32 bp, Artificial Sequence
  Synthesized
gaaatcaacttttgttcgcgctgtgggcctgc

SEQ ID NO: 120 - DNA, 37 bp, Artificial Sequence
  Synthesized
gacactttttcaaaatgacacctcctactgctgttga

SEQ ID NO: 121 - DNA, 35 bp, Artificial Sequence
  Synthesized
gaaatcaacttttgttcagcatcaggaccagcagc

SEQ ID NO: 122 - DNA, 38 bp, Artificial Sequence
  Synthesized
gacactttttcaaaatgcgcgtgaacaaaagtttaacc

SEQ ID NO: 123 - DNA, 40 bp, Artificial Sequence
  Synthesized
gaaatcaacttttgttccatcttcggacctgcttctacca

SEQ ID NO: 124 - DNA, 35 bp, Artificial Sequence
  Synthesized
gacactttttcaaaatgcatgccactggcttaact

SEQ ID NO: 125 - DNA, 33 bp, Artificial Sequence
  Synthesized
gaaatcaacttttgttcacgttgtgggccggca

SEQ ID NO: 126 - DNA, 42 bp, Artificial Sequence
  Synthesized
gacactttttcaaaatgtcaccaactaagacacaaaattctg

SEQ ID NO: 127 - DNA, 39 bp, Artificial Sequence
  Synthesized
gaaatcaacttttgttctaattgcggaccagcagctaag

SEQ ID NO: 128 - DNA, 32 bp, Artificial Sequence
  Synthesized
gacactttttcaaaatggcacccccagcagta

SEQ ID NO: 129 - DNA, 36 bp, Artificial Sequence
  Synthesized
gaaatcaacttttgttcaacatcgggtccagcagct

SEQ ID NO: 130 - DNA, 37 bp, Artificial Sequence
  Synthesized
gacactttttcaaaatgcagatcaacggtattacccc

SEQ ID NO: 131 - DNA, 32 bp, Artificial Sequence
  Synthesized
gaaatcaacttttgttcgcgcttcggccctgc

SEQ ID NO: 132 - DNA, 40 bp, Artificial Sequence
  Synthesized
gacactttttcaaaatgagcacgatgtgtgtcgataataa

SEQ ID NO: 133 - DNA, 41 bp, Artificial Sequence
  Synthesized
gaaatcaacttttgttcgtctaactgtggtccggctttaac

SEQ ID NO: 134 - DNA, 37 bp, Artificial Sequence
  Synthesized
gacactttttcaaaatgagcgctaaaagcattaccct

SEQ ID NO: 135 - DNA, 38 bp, Artificial Sequence
  Synthesized
gaaatcaacttttgttccagttttggccctgctttcac

SEQ ID NO: 136 - DNA, 42 bp, Artificial Sequence
  Synthesized
gacactttttcaaaatgtccccttctaaaatgaatgctacag

SEQ ID NO: 137 - DNA, 38 bp, Artificial Sequence
  Synthesized
gaaatcaacttttgttcctcgaattgaggaccagcggc

SEQ ID NO: 138 - DNA, 36 bp, Artificial Sequence
  Synthesized
gacactttttcaaaatgcgtgctaaaggtatcaccc

SEQ ID NO: 139 - DNA, 33 bp, Artificial Sequence
  Synthesized
gaaatcaacttttgttccagcttcggaccggcg

SEQ ID NO: 140 - DNA, 40 bp, Artificial Sequence
  Synthesized
gacactttttcaaaatgaccgttatggaacatactaaggc

SEQ ID NO: 141 - DNA, 38 bp, Artificial Sequence
  Synthesized
gaaatcaacttttgttcatctagctgaggacctgcagc

SEQ ID NO: 142 - DNA, 44 bp, Artificial Sequence
  Synthesized
gacactttttcaaaatggctccaactaaaatagaagattactct

SEQ ID NO: 143 - DNA, 35 bp, Artificial Sequence
  Synthesized
gaaatcaacttttgttctagggctggaccagctgc

SEQ ID NO: 144 - DNA, 40 bp, Artificial Sequence
  Synthesized
gacactttttcaaaatgaccgttatggaacatacaaaggc

SEQ ID NO: 145 - DNA, 37 bp, Artificial Sequence
  Synthesized
gaaatcaacttttgttcatctagctgaggtccagccg

SEQ ID NO: 146 - DNA, 39 bp, Artificial Sequence
  Synthesized
gacactttttcaaaatgagtgttaaaggaattaccccgc

SEQ ID NO: 147 - DNA, 35 bp, Artificial Sequence
  Synthesized
gaaatcaacttttgttcgatcttcggccctgcgct

SEQ ID NO: 148 - DNA, 33 bp, Artificial Sequence
  Synthesized
gacactttttcaaaatgtctccccccgtcaacc

SEQ ID NO: 149 - DNA, 33 bp, Artificial Sequence
  Synthesized
gaaatcaacttttgttcagccttagggccggca

SEQ ID NO: 150 - DNA, 39 bp, Artificial Sequence; identical to SEQ ID NO: 146
  Synthesized

SEQ ID NO: 151 - DNA, 37 bp, Artificial Sequence
  Synthesized
gaaatcaacttttgttcgacttttggcccagcactga

SEQ ID NO: 152 - DNA, 34 bp, Artificial Sequence
  Synthesized
gacactttttcaaaatgtcgccctccaaagttcc

SEQ ID NO: 153 - DNA, 40 bp, Artificial Sequence
  Synthesized
gaaatcaacttttgttcgaattgaggaccagctgcaagaa

SEQ ID NO: 154 - protein, 540 aa, Escherichia coli
  EcoPCK
MRVNNGLTPQELEAYGISDVHDIVYNPSYDLLYQEELDPSLTGYERGVLTNLGAVAVDTGIFTGRSPKDKYIVRDDTTRDTFWWADKGKGKNDNKPLSPETWQHLKGLVTKQLSGKRLFVVDAFCGANPDTRLSVRFITEVAWQAHFVKNMFIRPSDEELAGFKPDFIVMNGAKCTNPQWKEQGLNSENFVAFNLTERMQLIGGTWYGGEMKKGMFSMMNYLLPLKGIASMHCSANVGEKGDVAVFFGLSGTGKTTLSTDPKRRLIGDDEHGWDDDGVFNFEGGCYAKTIKLSKEAEPEIYNAIRRDALLENVTVREDGTIDFDDGSKTENTRVSYPIYHIDNIVKPVSKAGHATKVIFLTADAFGVLPPVSRLTADQTQYHFLSGFTAKLAGTERGITEPTPTFSACFGAAFLSLHPIQYAEVLVKRMQAAGAQAYLVNTGWNGTGKRISIKDTRAIIDAILNGSLDNAETFTLPMFNLAIPTELPGVDTKILDPRNTYASPEQWQEKAETLAKLFIDNFDKYTDTPAGAALVAAGPKL

SEQ ID NO: 155 - protein, 1178 aa, Gallus gallus
  misc_feature (566)..(566): Xaa can be any naturally occurring amino acid
MSQLCVPRGGRALLGAWRLPLLRPPPGSVRSASCQPIRKVLVANRGEIAIRVFRACTELGLRTVAVYSEQDTGQMHRQKADEAYLVGRGLPPVQAYLHVPDIIRVARENAVDAIHPGYGFLSERADFAQACVDAGVRFVGPPPEVVRKMGDKVEARSIAIAAGVPVVPGTSAPVATLGEAQDFAARVGFPIIFKAAHGGGGRGMRAVRGPQELEESFSRASSEALAAFGDGALFVEKLMERPRHIEGQILGDKHGNVVHLYERDCSIQRRHQKVVEIAPAARLDPQLRAQLASDAVRIAQQVGYENAGTVEFLVDRDGKHYFIEVNSRLQVEHTVTEEITGVDLVQAQLLVAAGRSLSELGLQQDSVRVNGCAIQCRVTTEDPARGFQPDTGRIEVFRSGEGMGIRLDGASAFQGALISPHYDSLLVKVIAHGPDQPSAAAKMSRALGEFRIRGVKTNIPFLQNVLAHPQFLGGAVDTQFIDENPELFHLRPSQNRAQKLLHYLGHVMVNGPSTPLPVKAKAAVVEPVPPPVPMGSPPEGLRAVLQREGPAGFARALRGHRGLLLXDTTFRDAHQSLLATRVRTRDLARIAPFVAHSLSPLCSMETWGGATFDVAMRFLHECPWERLRELRRLVPNIPFQMLLRGANAVGYTNYPDNVIYRFCEVAAANGMDIFRIFDALNYLPNLLLGVEAVGRAGAVVEAALSYTGDVADPTRTKYSLDYYLGLAKELVAAGTHILCIKDMAGLLTPAAARLLVSSLRDRFPDVPIHVHTHDTAGAAIATLLAAANADADVVDVAVDAMSGMTSQPSMGALVACARGTPLDTGIALERVFEYSEYWEGARGLYAAFDCTATMKSGNADVYENEIPGGQYTNLHFQAHAMGLGHKFKEVKKAYAEANKLLGDLIKVTPSSKVVGDLAQFMVQNGLSREEAEARADELSFPLSVVEFLQGYIGTPPGGFPEPFRSKVLKDLPRVEGRPGASLPPLDFEALSQELGARDGTPPSPEDLLSAALYPKVYAEFRDFTSTFGPVSCLGTRLFLEGPTIAEEFEVELERGKTLHIKALALGDLNAAGQREAFFELNGQLRSILVRDTQALKEMHVHPKADRSAKGQVGAPMPGEVVEVRVKEGEAVEKGAPLCVLSAMKMETVVTAPRGGTVSRLHVRPGMSLEGDDLIAEIE

SEQ ID NO: 156 - protein, 312 aa, Escherichia coli
  EcoMDH
MKVAVLGAAGGIGQALALLLKTQLPSGSELSLYDIAPVTPGVAVDLSHIPTAVKIKGFSGEDATPALEGADVVLISAGVARKPGMDRSDLFNVNAGIVKNLVQQVAKTCPKACIGIITNPVNTTVAIAAEVLKKAGVYDKNKLFGVTTLDIIRSNTFVAELKGKQPGEVEVPVIGGHSGVTILPLLSQVPGVSFTEQEVADLTKRIQNAGTEVVEAKAGGGSATLSMGQAAARFGLSLVRALQGEQGVVECAYVEGDGQYARFFSQPLLLGKNGVEERKSIGTLSAFEQNALEGMLDTLKKDIALGEEFVNK

SEQ ID NO: 157 - DNA, 38 bp, Artificial Sequence
  Synthesized
gacactttttcaaaatgcgcgttaacaatggtttgacc

SEQ ID NO: 158 - DNA, 32 bp, Artificial Sequence
  Synthesized
gaaatcaacttttgttccagcttcggaccagc

SEQ ID NO: 159 - DNA, 34 bp, Artificial Sequence
  Synthesized
catctttttcaaacatgtcgcagctgtgtgtccc

SEQ ID NO: 160 - DNA, 37 bp, Artificial Sequence
  Synthesized
atcatcatcatccttgtaatcctcgatctcggcgatg

SEQ ID NO: 161 - DNA, 32 bp, Artificial Sequence
  Synthesized
actttttcaaacatgaaagtcgcagtcctcgg

SEQ ID NO: 162 - DNA, 43 bp, Artificial Sequence
  Synthesized
atcatcatcatccttgtaatccttattaacgaactcttcgccc

(19) United States
(12) Patent Application Publication
(10) Pub. No.: US 2017/0253895 A1
(43) Pub. Date: Sep. 7, 2017

(54) TRANSFORMANT, METHOD FOR MANUFACTURING SAME, AND METHOD FOR MANUFACTURING DICARBOXYLIC ACID HAVING 4 CARBON ATOMS

(71) Applicant: JMTC Enzyme Corporation, Tokyo (JP)

(72) Inventors: Futoshi Hara, Yokohama-shi (JP); Shuichiro Kimura, Yokohama-shi (JP); Tetsuya Kotani, Yokohama-shi (JP); Takayuki Tanaka, Tokyo (JP)

(21) Appl. No.: 15/316,005

(22) PCT Filed: Jun. 26, 2015
(86) PCT No.: PCT/JP2015/068589

§ 371 Date: Dec. 2, 2016

(30) Foreign Application Priority Data
Jun. 30, 2014 (JP) 2014-135043

(51) Int. Cl. C12P 7/46; C12N 9/00; C12N 9/04; C12N 9/88

(52) U.S. Cl. CPC C12P 7/46; C12N 9/88; C12N 9/93; C12N 9/0006; C12Y 401/01031; C12Y 604/01001; C12Y 101/01037

(57) ABSTRACT

Provided is a transformant which uses Schizosaccharomyces pombe as a host, into which one or more foreign genes selected from the group consisting of a phosphoenolpyruvate carboxykinase gene and a pyruvate carboxylase gene are incorporated, and in which pdc2 genes of the Schizosaccharomyces pombe host have undergone deletion or deactivation, and a method for manufacturing a dicarboxylic acid having 4 carbon atoms by using the transformant.

### TECHNICAL FIELD

The present invention relates to a transformant, a method for manufacturing the same, and a method for manufacturing a dicarboxylic acid having 4 carbon atoms (C4 dicarboxylic acid). More specifically, the present invention relates to a transformant, which is obtained by incorporating one or more foreign genes of one or more kinds selected from the group consisting of a Phosphoenolpyruvate carboxykinase (hereinafter, referred to PCK as well) gene, a Pyruvate carboxylase (hereinafter, referred to as PYC as well) gene, and a Malate dehydrogenase (hereinafter, referred to as MDH as well) gene into Schizosaccharomyces pombe (hereinafter, referred to as S. pombe as well) and in which some of the genes in a group of Pyruvate decarboxylase (hereinafter, referred to as PDC as well)-encoding genes and Malic enzyme (hereinafter, referred to as MAE as well) genes have undergone deletion or deactivation, a method for manufacturing the transformant, and a method for manufacturing a C4 dicarboxylic acid in which the transformant is cultured in a culture solution and a C4 dicarboxylic acid is obtained from the culture solution.

### BACKGROUND ART

Malic acid (HOOCCH(OH)CH₂COOH) is a dicarboxylic acid consisting of 4 carbon atoms (C4 dicarboxylic acid). Generally, malic acid is commercially manufactured through chemical synthesis from raw materials derived from petroleum or through microbial fermentation from renewable feedstocks.

Many natural or genetic recombinant microorganisms are known to be able to produce malic acid as the main fermentative organisms. For example, as a method of using wild-type microorganisms, a method of producing malic acid by culturing Aspergillus flavus (Non-Patent Document 1) and Penicillium sclerotiorum (Non-Patent Document 2) has been disclosed.

Regarding a microorganism whose malic acid producibility is improved by gene recombination, Patent Document 1 describes that a C4 dicarboxylic acid producibility is improved by introducing a C4 dicarboxylic acid transporter into filamentous bacteria such as Aspergillus oryzae.

In Patent Document 2, malic acid is produced using a transformant which is obtained by deleting a PDC gene from Saccharomyces cerevisiae and introducing a PYC gene or a Phosphoenolpyruvate carboxykinase (hereinafter, referred to PCK as well) gene, an MDH gene, and a malic acid transporter protein gene into the Saccharomyces cerevisiae.

In Non-Patent Document 3, malic acid is produced using a transformant which is obtained by deleting a PDC gene from Saccharomyces cerevisiae and introducing a PYC gene, an MDH gene, and a malic acid transporter protein gene into the Saccharomyces cerevisiae. In Patent Document 3, malic acid is produced using a transformant which is obtained by deleting a PDC enzyme gene, a pyruvate kinase (PYK) gene, and a hexokinase (HXK) gene from Saccharomyces cerevisiae and introducing a PYC gene, an MDH gene, a malic acid transporter protein gene, and a PCK gene into the Saccharomyces cerevisiae.

In Non-Patent Document 4, a transformant is manufactured whose malic acid producibility is improved by deactivating a plurality of enzymes in E. coli that is involved in pathways other than a pathway through which malic acid is produced from pyruvic acid. Non-Patent Document 4 discloses that between a deletion strain of a malic enzyme gene maeB and a non-deletion strain thereof, the maeB gene non-deletion strain has a higher malic acid production rate. The maeB gene of E. coli corresponds to a malic enzyme gene mae2 of S. Pombe.

### CITATION LIST

### Patent Literature

[Patent Document 1] Published Japanese Translation No. 2013-503631 of the PCT International Publication

[Patent Document 2] Published Japanese Translation No. 2009-516526 of the PCT International Publication

[Patent Document 3] PCT International Publication No. WO2009/011974

### Non-Patent Literature

[Non-Patent Document 1] Battat et al., Biotechnology and Bioengineering, 1991, vol. 37, p. 1108-1116.

[Non-Patent Document 2] Wang et al., Bioresource Technology, 2013, vol. 143, p. 674-677.

[Non-Patent Document 3] Zelle et al., Applied and Environmental Microbiology, 2008, vol. 74, p. 2766-2777.

[Non-Patent Document 4] Zhang et al., Applied and Environmental Microbiology, 2011, vol. 77, p. 427-434.

### SUMMARY OF INVENTION

### Technical Problem

All of the transformants disclosed in Patent Document 1 to 3 and Non-Patent Document 3 and 4 have a malic acid production rate of less than 1.0 g/(L·h). Therefore, the development of a microorganism having higher malic acid producibility is desired.

An object of the present invention is to provide a Schizosaccharomyces pombe transformant, which can excellently produce a C4 dicarboxylic acid through microbial fermentation from renewable feedstocks, and a method for manufacturing the transformant.

Another object of the present invention is to provide a method for manufacturing a C4 dicarboxylic acid including malic acid by using the transformant.

### Solution to Problem

A transformant according to the present invention is a transformant which uses Schizosaccharomyces pombe as a host and, into which one or more foreign genes of one or more kinds selected from the group consisting of a phosphoenolpyruvate carboxykinase gene and a pyruvate carboxylase gene are incorporated, and in which some of the genes in a group of pyruvate decarboxylase-encoding genes of the Schizosaccharomyces pombe host have undergone deletion or deactivation, the Phosphoenolpyruvate carboxykinase gene encodes a polypeptide which includes an amino acid sequence represented by any of SEQ ID NOS: 94 to 113, a polypeptide which includes an amino acid sequence obtained by substitution, addition, or deletion of one or a plurality of amino acids in an amino acid sequence represented by any of SEQ ID NOS: 94 to 113 and has a phosphoenolpyruvate carboxykinase activity, or a polypeptide which includes an amino acid sequence sharing a sequence identity of equal to or higher than 80% with an amino acid sequence represented by any of SEQ ID NOS: 94 to 113 and has phosphoenolpyruvate carboxykinase activity, the pyruvate carboxylase gene encodes a polypeptide which includes an amino acid sequence represented by any of SEQ ID NOS: 1 to 16, a polypeptide which includes an amino acid sequence obtained by the substitution, addition, or deletion of one or a plurality of amino acids in an amino acid sequence represented by any of SEQ ID NOS: 1 to 16 and has pyruvate carboxylase activity, or a polypeptide which includes an amino acid sequence sharing a sequence identity of equal to or higher than 80% with an amino acid sequence represented by any of SEQ ID NOS: 1 to 16 and has pyruvate carboxylase activity, and the pyruvate decarboxylase-endocing genes that have undergone deletion or deactivation are pdc2 genes.

It is preferable that one or more foreign malate dehydrogenase genes are further incorporated into the transformant according to the present invention.

It is preferable that in the transformant according to the present invention, the malate dehydrogenase genes are genes encoding a polypeptide which includes an amino acid sequence represented by any of SEQ ID NOS: 17 to 21, a polypeptide which includes an amino acid sequence obtained by the substitution, addition, or deletion of one or a plurality of amino acids in an amino acid sequence represented by any of SEQ ID NOS: 17 to 21 and has malate dehydrogenase activity, or a polypeptide which includes an amino acid sequence sharing sequence identity of equal to or higher than 80% with an amino acid sequence represented by any of SEQ ID NOS: 17 to 21 and has malate dehydrogenase activity.

It is preferable that in the transformant according to the present invention, malic enzyme genes have also undergone deletion or deactivation.

It is preferable that in the transformant according to the present invention, one or more foreign phosphoenolpyruvate carboxykinase genes or one or more foreign pyruvate carboxylase genes and one or more foreign malate dehydrogenase genes are incorporated, and all of the pdc2 genes and the malic enzyme gene have undergone deletion or deactivation.

It is preferable that in the transformant according to the present invention, the foreign genes are incorporated into a chromosome of the host.

A method for manufacturing a dicarboxylic acid having 4 carbon atoms according to the present invention includes culturing the transformant in a culture solution and obtaining a dicarboxylic acid having 4 carbon atoms from the cultured transformant or a culture supernatant.

It is preferable that in the method for manufacturing a dicarboxylic acid having 4 carbon atoms according to the present invention, the dicarboxylic acid having 4 carbon atoms is malic acid or oxaloacetic acid.

It is preferable that in the method for manufacturing a dicarboxylic acid having 4 carbon atoms according to the present invention, culturing is continued even after the pH of the culture solution becomes equal to or less than 3.5.

### Advantageous Effects of Invention

The schizosaccharomyces pombe transformant according to the present invention can produce a C4 dicarboxylic acid including malic acid at a high production rate that was not achieved in the related art.

The transformant can be obtained in a more simple manner by the method for manufacturing a transformant according to the present invention.

The method for manufacturing a C4 dicarboxylic acid according to the present invention makes it possible to manufacture a C4 dicarboxylic acid with higher productivity through microbial fermentation.

### BRIEF DESCRIPTION OF DRAWINGS

FIG. 1 is a view showing a constitution of a monodentate integrative recombinant vector pSMh.

FIG. 2 is a view showing relative values of a PYC expression amount of 16 kinds of transformant into which a PYC gene is introduced.

FIG. 3 is a view showing relative PYC activity per enzyme liquid (mU/mL) of 8 kinds of transformant into which a PYC gene is introduced.

FIG. 4 is a view showing relative values of an MDH expression amount of 5 kinds of transformant into which an MDH gene is introduced.

FIG. 5 is a view showing MDH activity per enzyme liquid (mU/mL) of 3 kinds of transformant into which an MDH gene is introduced.

FIG. 6 is a view showing how a glucose concentration (g/L), an ethanol concentration (g/L), and a malic acid concentration (g/L) of a wild-type strain (ARC010 strain) change over time.

FIG. 7 is a view showing how a glucose concentration (g/L), an ethanol concentration (g/L), and a malic acid concentration (g/L) of an ASP4590 strain (Δpdc2) change over time.

FIG. 8 is a view showing how a glucose concentration (g/L), an ethanol concentration (g/L), and a malic acid concentration (g/L) of an ASP4491 strain (Δpdc2, +ScePYC, +DacMDH) change over time.

FIG. 9 is a view showing how a glucose concentration (g/L), an ethanol concentration (g/L), and a malic acid concentration (g/L) of an ASP4964 strain (Δpdc2, Δmae2, +ScePYC, +DacMDH) change over time.

FIG. 10 is a view showing how a glucose concentration (g/L), an ethanol concentration (g/L), and a malic acid concentration (g/L) of an ASP4933 strain (Δpdc2, Δfum1, +ScePYC, +DacMDH) change over time.

FIG. 11 is a view how a glucose concentration (g/L) and a malic acid concentration (g/L) of a culture solution of each of an ASP4491 strain and an ASP4892 strain change over time.

FIG. 12 is a view showing relative values of a PCK expression amount of 20 kinds of transformant into which a PCK gene is introduced.

### DESCRIPTION OF EMBODIMENTS

[Gene Recombinant]

The transformant according to the present invention is a transformant which uses schizosaccharomyces pombe as a host, into which one or more foreign genes selected from the group consisting of a phosphoenolpyruvate carboxykinase (PCK) gene and a pyruvate carboxylase (PYC) gene are incorporated, and in which some of the genes in gene group pyruvate decarboxylase-encoding genes of the schizosaccharomyces pombe host have undergone deletion or deactivation.

In the present invention and the specification of the present application, a foreign gene means a structural gene which is not a structure gene inherent to a host (a structural gene contained in a chromosome of a natural-type host having not undergone transformation) but a structural gene introduced into a host through a transformation operation or the like. A gene inherent to a host is also included in a foreign gene of the present invention as long as the gene is introduced into the host through a transformation operation or the like.

Oxaloacetic acid is a sort of C4 dicarboxylic acid. In S. pombe, oxaloacetic acid is important as a raw material for the synthesis of other C4 dicarboxylic acids such as malic acid. In wild-type S. pombe, oxaloacetic acid is synthesized mainly through a pathway in which oxaloacetic acid is directly synthesized from phosphoenolpyruvic acid by PCK and synthesized by PYC through pyruvic acid. Therefore, if PCK activity and PYC activity are enhanced, an amount of oxaloacetic acid produced from S. pombe could be increased.

Pyruvic acid is important in ethanol fermentation. Pyruvic acid is converted into acetaldehyde by pyruvate decarboxylase, and then the acetaldehyde is converted into ethanol by alcohol dehydrogenase. That is, because pyruvic acid is used as a raw material of ethanol fermentation, in order to increase an amount of oxaloacetic acid produced, not only the enhancement of PCK activity and PYC activity, but also the inhibition of ethanol fermentation needs to be achieved.

Into the transformant according to the present invention, at least one of the foreign genes including the PCK gene and the PYC gene is incorporated. Furthermore, in the transformant according to the present invention, at least one of the PCK activity and the PYC activity is enhanced, some or the genes in a group of pyruvate decarboxylase-encoding genes have undergone deletion or deactivation, and ethanol fermentation efficiency is reduced.

That is, in the transformant according to the present invention, ethanol fermentation is inhibited, and an amount of pyruvic acid usable as a matrix of PYC is increased. Therefore, the transformant has excellent oxaloacetic acid producibility and excellent producibility of other C4 dicarboxylic acids synthesized from oxaloacetic acid.

In wild-type S. pombe, through a pathway in which aerobic pyruvic acid is converted into acetyl CoA, citric acid, succinic acid, and the like, malic acid is produced through fumaric acid hydration by fumarate dehydrase. Oxaloacetic acid is produced by anaerobic PYC, and malic acid is produced from the oxaloacetic acid by malate dehydrogenase (MDH). In the transformant according to the present invention, an amount of oxaloacetic acid produced is increased. Therefore, the transformant also has excellent malic acid producibility.

Into the transformant according to the present invention, at least one PCK gene or at least one PYC gene should be introduced as a foreign gene. The number of PYC genes to be introduced is not limited to 1 and may be equal to or greater than 2. The more the foreign genes to be introduced, the higher then PYC activity of the obtained transformant.

In a case where 2 or more PYC genes are introduced, PYC genes of the same kind may be introduced, or two or more kinds of PYC genes may be introduced. Similarly, into the transformant according to the present invention, 1 PCK gene may be introduced, or 2 or more PCK genes of the same kind or different kinds may be introduced.

In order to cause the transformant according to the present invention to have particularly high malic acid producibility, it is preferable to inhibit the decomposition of malic acid by enhancing MDH activity.

Specifically, a transformant is preferable which is obtained by incorporating at least one of the foreign genes including a PCK gene and a PYC gene and one or more foreign MDH genes into S. pombe as a host, and in which some of the genes in a group of pyruvate decarboxylase-encoding genes and malic enzyme genes have undergone deletion or deactivation. The aforementioned transformant is a transformant into which foreign genes such as at least either the PCK gene or the PYC gene and the MDH genes are incorporated, and in which at least either the PCK activity or the PYC activity and the MDH activity are enhanced, some of the genes in a group pyruvate decarboxylase-encoding genes have undergone deletion or deactivation, and ethanol fermentation efficiency is reduced. That is, in the transformant, malic acid fermentation is accelerated while ethanol fermentation is inhibited. Therefore, the transformant has excellent malic acid producibility.

In a case where at least 1 MDH gene is introduced as a foreign gene into the transformant according to the present invention, 1 MDH gene or 2 or more MDH genes of the same kind or different kinds may be introduced into the transformant according to the present invention.

It is preferable that at least 1 PCK gene, at least 1 PYC gene, and at least 1 MDH gene are introduced as foreign genes into the transformant according to the present invention. By the incorporation of foreign genes such as the PCK gene, the PYC gene, and the MDH gene, malic acid producibility is further improved.

<S. pombe>

S. pombe used as a host is yeast (fission yeast) of the genus Schizosaccharomyces, and is a microorganism having particularly excellent acid resistance compared to other yeasts. Because S. pombe inherently has an mae1 (C4 dicarboxylic acid transporter) gene, even in a case where an amount of C4 dicarboxylic acid produced in the microbial cells is increased, an excess of C4 dicarboxylic acid is inhibited from affecting the growth or the like of the microbial cells. Therefore, compared to other yeasts not having the Mae1 gene, such as Saccharomyces cerevisiae, S. pombe is suitable for producing a C4 dicarboxylic acid.

The entire base sequence of a chromosomes of S. pombe has been publicized by being listed as “Schizosaccharomyces pombe Gene DB (http://www.genedb.org/genedb/pombe/)” in the database “Gene DB” of Sanger Institute. The gene sequence data of S. pombe described in the present specification can be obtained through the search by the gene name or the aforementioned strain name from the database described above.

<Pyruvate Decarboxylase-Encoding Gene>

The group of pyruvate decarboxylase-encoding genes (pyruvate decarboxylase genes, hereinafter, referred to as “PDC genes” as well) in S. pombe consists of 4 kinds of genes including a gene encoding pyruvate decarboxylase 1 (hereinafter, referred to as a “PDC1 gene”), a gene encoding pyruvate decarboxylase 2 (hereinafter, referred to as a “PDC2 gene”), a gene encoding pyruvate decarboxylase 3 (hereinafter, referred to as a “PDC3 gene”), and a gene encoding pyruvate decarboxylase 4 (hereinafter, referred to as a “PDC4 gene”). Among these, the PDC2 gene and the PDC4 gene are PDC genes that play a key functional role in S. pombe.

The strain name of each of the PDC genes is as follows.

PDC1 gene (pdc1); SPAC13A11. 06

PDC2 gene (pdc2); SPAC1F8. 07c

PDC3 gene (pdc3); SPAC186. 09

PDC4 gene (pdc4); SPAC3G9. 11c

The PDC gene sequence data can be obtained through the search by the gene name or the strain name from the aforementioned S. pombe gene database.

The transformant according to the present invention has a chromosome in which some of the genes in a group of pyruvate decarboxylase-encoding genes have undergone deletion or deactivation. Due to the deletion or deactivation of some of the genes in the group of PDC genes of the transformant, the ethanol fermentation efficiency of the transformant is reduced, and the amount of pyruvic acid to be converted into ethanol is decreased. Therefore, the productivity of a C4 dicarboxylic acid including malic acid is improved. Here, if the group of PDC genes is totally deleted or deactivated, ethanol fermentation is not performed at all, and the growth of the transformant is inhibited. Accordingly, only some of the genes in the group of PDC genes should be deleted or deactivated.

The PDC genes to be deleted or deactivated are particularly preferably PDC2 genes. The PDC2 genes are PDC genes that particularly play a key functional role. An amino acid sequence of PDC2 (SpoPDC2) encoded by the PDC2 genes of S. pombe is represented by SEQ ID NO: 22.

As described above, if all of the PDC genes are deleted or deactivated, the transformant does not perform ethanol fermentation, and thus the growth of the transformant is hindered. Therefore, the deletion or deactivation of the PDC genes must be performed by maintaining an ethanol fermentation ability necessary for the growth so as to obtain a sufficient amount of transformant and simultaneously by lowering the ethanol fermentation ability so as to improve fermentation efficiency of a C4 dicarboxylic acid.

In order to delete or deactivate the PDC genes as described above, the inventors of the present invention conducted investigation. As a result, they found that if PDC2 genes are deleted or deactivated, PDC4 genes are activated to some extent, and the ethanol fermentation ability enough for obtaining a sufficient amount of transformant and the production of a C4 dicarboxylic acid with high fermentation efficiency can be accomplished simultaneously.

<Malic Enzyme Gene>

A malic enzyme-endcoding gene (malic enzyme gene, hereinafter, referred to as an “mae gene” as well) in S. pombe is a gene encoding malic enzyme 2 (hereinafter, referred to as an “mae2 gene”). The strain name of the mae2 gene is as follows.

mae2 gene (mae2); SPCC794. 12c

The mae2 gene sequence data can be obtained through the search by the gene name or the strain name from the aforementioned S. pombe gene database. An amino acid sequence of mae2 (Spomae2) encoded by the mae2 gene of S. pombe is represented by SEQ ID NO: 23.

In the wild-type S. pombe, some of malic acids produced are converted into pyruvic acid by mae2. In the transformant according to the present invention in which the mae2 gene has undergone deletion or deactivation, malic acid produced in the transformant is not converted into pyruvic acid, and hence the amount of malic acid produced significantly increases.

<Deletion or Deactivation of Gene>

The deletion or deactivation of the PDC genes and the mae2 genes can be performed by a known method. For example, by using a Latour method (described in the journal of Nucleic Acids Research, 2006, Vol. 34, p. e11, PCT International Publication No. WO2007/063919, and the like), the PDC genes and the like can be deleted.

Furthermore, through the deletion, insertion, substitution, or addition induced in a portion of a base sequence of the PDC genes and the like, the PDC genes and the like can be deactivated. Only one of the mutations including deletion, insertion, substitution, and addition may be induced, or two or more mutations among the above may be induced.

As the method for introducing the mutation into a portion of the PDC genes and the like, a known method can be used.

For example, a mutation separation method using a mutagen (Experimental Method of Yeast Molecular Genetics, 1996, Gakkai Shuppan Center) and a random mutation method using a polymerase chain reaction (PCR) (the journal of PCR Methods Application, Vol. 2, pp. 28-33, 1992) can be used.

The PDC genes that carry the mutation introduced into a portion thereof may be genes expressing temperature-sensitive mutant-type pyruvate decarboxylase. The temperature-sensitive mutant-type pyruvate decarboxylase is an enzyme which shows activity equivalent to the activity of wild-type pyruvate decarboxylase at a certain culture temperature but undergoes the loss or deterioration of the activity at a temperature equal to or higher than a specific culture temperature.

A mutant strain expressing the mutant-type pyruvate decarboxylase can be obtained by being selected from strains whose growth rate is equivalent to the growth rate of the wild-type yeast under the conditions in which the activity is not limited by the temperature but is greatly reduced under specific temperature conditions in which the activity is limited.

Similarly, the mae2 genes that carry the mutation introduced into a portion thereof may be genes expressing temperature-sensitive mutant-type mae2.

The deletion or deactivation of genes does not mean only the deletion or deactivation of structural genes. Not only a case where structural genes are deleted, but also a case where even if a protein is encoded by a structural gene and the structural gene is expressed, the protein is not an enzyme having activity means that the genes are deactivated.

In a case where even if a structural domain of a gene encodes an enzyme having activity, a protein of the structural domain of the gene is not expressed due to the deletion of genes encoding a regulatory domain or the mutation of the sequence of the genes, it means that the genes are “deleted or deactivated”. Therefore, the PDC genes or the mae2 genes to be deleted or deactivated may be either or both of a structural domain and a regulatory domain of the PDC genes or the mae2 genes.

<PYC Gene>

The PYC genes introduced as foreign genes into the transformant according to the present invention should be structural genes which can express a protein performing PYC activity in a case where the structural genes are introduced into S. pombe, and may be derived from any of the biospecies.

Examples of PYC encoded by the PYC genes introduced as foreign genes include PYC derived from Aspergillus niger (AniPYC) (AC. No.: CAC19838. 1.) (SEQ ID NO: 1), PYC derived from Brevibacillus brevis (BbrPYC) (SEQ ID NO: 2), PYC derived from Debaryomyces hansenii (DhaPYC) (AC. No.: CAG86153. 2.) (SEQ ID NO: 3), PYC derived from Kluyveromyces lactis (KlaPYC) (SEQ ID NO: 4), PYC derived from Lachancea thermotolerans (LthPYC) (SEQ ID NO: 5), PYC derived from Lodderomyces elongisporus (LelPYC) (SEQ ID NO: 6), PYC derived from Saccharomyces cerevisiae (ScePYC) (SEQ ID NO: 7), PYC derived from Candida orthopsilosis (CorPYC) (AC. No.: CCG23801. 1.) (SEQ ID NO: 8), PYC derived from Candida tropicalis (CtrPYC) (AC. No.: EER35520. 1.) (SEQ ID NO: 9), PYC derived from Naumovozyma castellii (NcaPYC) (AC. No.: CCC71457. 1.) (SEQ ID NO: 10), PYC derived from Naumovozyma dairenensis (NdaPYC) (AC. No.: CCD26740. 1.) (SEQ ID NO: 11), PYC derived from Saccharomyces kudriavzevii (SkuPYC) (AC. No.: EJT41260. 1.) (SEQ ID NO: 12), PYC derived from S. pombe (SpoPYC) (AC. No.: BAA11239. 1, CAB52809. 1.) (SEQ ID NO: 13), PYC derived from Tetrapisispora blattae (TblPYC) (AC. No.: CCH58779. 1.) (SEQ ID NO: 14), PYC derived from Torulaspora delbrueckii (TdePYC) (AC. No.: CCE93722. 1.) (SEQ ID NO: 15), and PYC derived from Zygosaccharomyces rouxii (ZroPYC) (AC. No.: CAR26934. 1.) (SEQ ID NO: 16).

The PYC genes may be genes encoding a polypeptide which includes an amino acid sequence obtained by the substitution, addition, or deletion of one or a plurality of amino acids in amino acid sequences (SEQ ID NOS: 1 to 16) of the above PYCs and has PYC activity.

The PYC genes may be genes encoding a polypeptide which includes an amino acid sequence sharing a sequence identity of equal to or higher than 80%, preferably equal to or higher than 85%, more preferably equal to or higher than 90%, and even more preferably equal to or higher than 95% with the amino acid sequences (SEQ ID NOS: 1 to 16) of the above PYCs and has PYC activity.

In the specification of the present application, “AC. No.” means an accession number of a database GenBank of National Center for Biotechnology Information (NCBI).

In the present invention and the specification of the present application, “plurality of amino acids” mean 2 to 20 amino acids and are preferably 2 to 10 amino acids.

The PYC genes introduced into the transformant according to the present invention are preferably genes encoding PYC which is the same as BbrPYC, KlaPYC, LelPYC, ScePYC, SpoPYC, or TblPYC or has an amino acid sequence similar to that of the above PYCs.

Specifically, the PYC genes are preferably genes encoding a polypeptide which includes amino acid sequences represented by SEQ ID NOS: 2, 4 to 7, and 12 to 14, a polypeptide which includes an amino acid sequence obtained by the substitution, addition, or deletion of one or a plurality of amino acids in amino acid sequences represented by SEQ ID NOS: 2, 4 to 7, and 12 to 14 and has PYC activity, or a polypeptide which includes an amino acid sequence sharing a sequence identity of equal to or higher than 80% with amino acid sequences represented by SEQ ID NOS: 2, 4 to 7, and 12 to 14 and has PYC activity.

The PYC genes are more preferably a polypeptide which is represented by SEQ ID NO: 7, a polypeptide which includes an amino acid sequence obtained by the substitution, addition, or deletion of one or a plurality of amino acids in an amino acid sequence represented by SEQ ID NO: 7 and has PYC activity, or a polypeptide which includes an amino acid sequence sharing a sequence identity of equal to or higher than 80% with an amino acid sequence represented by SEQ ID NO: 7 and has PYC activity. The PYC genes are even more preferably a polypeptide which is represented by SEQ ID NO: 7 or a polypeptide which includes an amino acid sequence obtained by the substitution, addition, or deletion of one or a plurality of amino acids in an amino acid sequence represented by SEQ ID NO: 7 and has PYC activity.

<MDH Gene>

The MDH genes introduced as foreign genes into the transformant according to the present invention should be structural genes which can express a protein performing MDH activity in a case where the structural genes are introduced into S. pombe, and may be derived from any biospecies.

Examples of MDH encoded by the MDH genes introduced as foreign genes include MDH derived from Archaeoglobus fulgidus (AfuMDH) (SEQ ID NO: 17), MDH derived from Congregibacter litoralis (CliMDH) (SEQ ID NO: 18), MDH derived from Delftia acidovorans (DacMDH) (SEQ ID NO: 19), MDH derived from Halomonas elongata (HelMDH) (SEQ ID NO: 20), and MDH derived from Shewanella putrefaciens (SpuMDH) (SEQ ID NO: 21).

The MDH genes may be genes encoding a polypeptide which includes an amino acid sequence obtained by the substitution, addition, or deletion of one or a aplurality of amino acids in amino acid sequences (SEQ ID NOS: 17 to 21) of the above MDHs and has MDH activity. Furthermore, the MDH genes may be genes encoding a polypeptide which includes an amino acid sequence sharing a sequence identity of equal to or higher than 80%, preferably equal to or higher than 85%, more preferably equal to or higher than 90%, and even more preferably equal to or higher than 95% with amino acid sequences (SEQ ID NOS: 17 to 21) of the above MDHs and has MDH activity.

The MDH genes introduced into the transformant according to the present invention are preferably genes encoding MDH which is the same as CliMDH, DacMDH, or HelMDH or has an amino acid sequence similar to that of the above MDHs.

Specifically, the MDH genes are preferably genes encoding a polypeptide which includes amino acid sequences represented by SEQ ID NOS: 18 to 20, a polypeptide which includes an amino acid sequence obtained by the substitution, addition, or deletion of one or a plurality of amino acids in amino acid sequences represented by SEQ ID NOS: 18 to 20 and has MDH activity, or a polypeptide which includes an amino acid sequence sharing a sequence identity of equal to or higher than 80% with amino acid sequences represented by SEQ ID NOS: 18 to 20 and has MDH activity.

The MDH genes are more preferably genes encoding a polypeptide represented by SEQ ID NO: 19, a polypeptide which includes an amino acid sequence obtained by the substitution, addition, or deletion of one or a plurality of amino acids in an amino acid sequence represented by SEQ ID NO: 19 and has MDH activity, or a polypeptide which includes an amino acid sequence sharing a sequence identity of equal to or higher than 80% with an amino acid sequence represented by SEQ ID NO: 19 and has MDH activity. The MDH genes are even more preferably a polypeptide represented by SEQ ID NO: 19 or a polypeptide which includes an amino acid sequence obtained by the substitution, addition, or deletion of one or a plurality of amino acids in an amino acid sequence represented by SEQ ID NO: 19 and has MDH activity.

<PCK Genes>

The PCK genes introduced as foreign genes into the transformant according the present invention should be structural genes which can express a protein performing PCK activity in a case where the structural genes are introduced into S. pombe, and may be derived from any biospecies.

Examples of PCK encoded by the PCK genes introduced as foreign genes include PCK derived from Candida glabrata (CglPCK) (AC. No.: CAG60017. 1) (SEQ ID NO: 94), PCK derived from Citrobacter koseri (CkoPCK) (AC. No.: KGY18702. 1) (SEQ ID NO: 95), PCK derived from Cronobacter sakazakii (CsaPCK) (AC. No.: EGL73852. 1) (SEQ ID NO: 96), PCK derived from Debaryomyces hansenii (DhaPCK) (AC. No.: CAG88379. 1) (SEQ ID NO: 97), PCK derived from Escherichia fergusonii (EfePCK) (AC. No.: EGC96802. 1) (SEQ ID NO: 98), PCK derived from Edwardsiella tarda (EtaPCK) (Ac. No.: GAC66070. 1) (SEQ ID NO: 99), PCK derived from Kluyveromyces lactis (KlaPCK) (AC. No.: AAC27661. 1) (SEQ ID NO: 100), PCK derived from Lodderomyces elongisporus (LelPCK) (Ac. No.: EDK41877. 1) (SEQ ID NO: 101), PCK derived from Pectobacterium carotovorum (PcaPCK) (Ac. No.: ACT14911. 1) (SEQ ID NO: 102), PCK derived from Photobacterium leiognathi (PlePCK) (Ac. No.: GAA03015. 1) (SEQ ID NO: 103), PCK derived from Providencia rettgeri (PrePCK) (Ac. No.: EFE52670. 1) (SEQ ID NO: 104), PCK derived from Saccharomyces cerevisiae (ScePCK) (Ac. No.: CAA31488. 1) (SEQ ID NO: 105), PCK derived from Serratia odorifera (SodPCK) (Ac. No.: EFE97343. 1) (SEQ ID NO: 106), PCK derived from Vibrio orientalis (VorPCK) (Ac. No.: EGU52194. 1) (SEQ ID NO: 107), PCK derived from Vanderwaltozyma polyspora (VpoPCK) (Ac. No.: EDO17884. 1) (SEQ ID NO: 108), PCK derived from Vibrio tubiashii (VtuPCK) (Ac. No.: EIF04922. 1) (SEQ ID NO: 109), PCK derived from Yersinia bercovieri (YbePCK) (Ac. No.: EEQ07887. 1) (SEQ ID NO: 110), PCK derived from Yarrowia lipolytica (YliPCK) (Ac. No.: CAG82248. 1) (SEQ ID NO: 111), PCK derived from Yersinia rohdei (YroPCK) (Ac. No.: AJJ11585. 1) (SEQ ID NO: 112), and PCK derived from Zygosaccharomyces rouxii (ZroPCK) (Ac. No.: CAR26716. 1) (SEQ ID NO: 113).

The PCK genes may be genes encoding a polypeptide which includes an amino acid sequence obtained by the substitution, addition, or deletion of one or a plurality of amino acids in amino acid sequences (SEQ ID NOS: 94 to 113) of the above PCKs and has PCK activity. Furthermore, the PCK genes may be genes encoding a polypeptide which includes an amino acid sequence sharing a sequence identity of equal to or higher than 80%, preferably equal to or higher than 85%, more preferably equal to or higher than 90%, and even more preferably equal to or higher than 95% with amino acid sequences (SEQ ID NOS: 94 to 113) of the above PCKs and has PCK activity.

As PCK, PYC, and MDH having high activity, PCK derived from Escherichia coli (EcoPCK) (AC. No.: AAA58200. 1) (SEQ ID NO: 154), PYC derived from Gallus gallus (GglPYC) (AC. No.: AAM92771. 1.) (SEQ ID NO: 155), and MDH derived from Escherichia coli (EcoMDH) (AC. No.: AAA58038. 1) (SEQ ID NO: 156) are known.

[Manufacturing of Transformant]

The transformant according to the present invention is obtained by using S. pombe, in which some of the genes in a group of PDC genes have undergone deletion or deactivation, as a host and introducing at least one of the foreign genes including a PCK gene and a PYC gene into the host by a genetic engineering method. Furthermore, the transformant according to the present invention can also be obtained by deleting or deactivating some of the genes in a group of PDC genes of a transformant which is obtained by introducing at least one of the foreign genes including a PCK gene and a PYC gene into S. pombe used as a host by a genetic engineering method.

The transformant according to the present invention into which foreign MDH genes are introduced and in which mae2 genes have undergone deletion or the like can be obtained by using S. pombe, in which some of the genes in a group of PDC genes have undergone deletion or deactivation, as a host, introducing at least one of the foreign genes including a PCK gene and a PYC gene and at least one foreign MDH gene into the host by a genetic engineering method, and deleting or deactivating an mae2 gene of the host.

In addition, S. pombe in which some of the genes in a group of PDC genes have undergone deletion or deactivation may be used as a host, an mae2 gene of the host may be deleted or deactivated, and then at least one of the foreign genes including a PCK gene and a PYC gene and at least one foreign MDH gene may be incorporated into the host by a genetic engineering method.

Furthermore, the transformant according to the present invention can also be obtained by deleting or deactivating some of the genes in a group of PDC genes and an mae2 gene of a transformant which is obtained by introducing at least one of the foreign genes including a PCK gene and a PYC gene and at least one foreign MDH gene into S. pombe used as a host by genetic engineering method. In a case where a plurality of foreign genes is introduced, all of the foreign genes may be introduced into the host at the same time or sequentially introduced into the host (in random order).

The method for manufacturing the transformant will be described based on, for example, a method of using S. pombe, in which some of the genes in a group of PDC genes have undergone deletion or deactivation, as a host, introducing a PYC gene and an MDH gene into the host, and then deleting or deactivating an mae2 gene.

<Host>

The S. pombe used as a host may be a wild type or a mutant type in which specific genes have undergone deletion or deactivation according to the purpose. As a method for deleting or deactivating the specific genes, a known method can be used. Specifically, by using the Latour method described above, the genes can be deleted.

Furthermore, by a mutation separation method using a mutagen (Experimental Method of Yeast Molecular Genetics, 1996, Gakkai Shuppan Center), a random mutation method using PCR (the journal of PCR Methods Application, Vol. 2, pp. 28-33, 1992), and the like, a mutation is introduced into some of the genes, thereby deactivating the genes. The yeast host of the genus Schizosaccharomyces in which specific genes have undergone deletion or deactivation is described in, for example, PCT International Publication No. WO2002/101038 and PCT International Publication No. WO2007/015470.

The portion in which the deletion or deactivation of specific genes is performed may be an open reading frame (ORF) portion or an expression regulatory sequence portion. It is particularly preferable to use a deletion or deactivation method by a PCR-mediated homologous recombination method (the journal of Yeast, Vol. 14, pp. 943-951, 1998) in which an ORF portion of a structural gene is substituted with a marker gene.

A mutant in which PDC genes have undergone deletion or deactivation can be preferably used as a host for manufacturing the transformant according to the present invention. Furthermore, the S. pombe, in which the PDC genes and specific genes other than PDC genes have undergone deletion or deactivation, can also be used as a host. By the deletion or deactivation of a protease gene and the like, the expression efficiency of heterologous proteins can be improved, and if the host obtained in this way is used as a host in the present invention, the production efficiency of a C4 dicarboxylic acid such as malic acid could be improved.

As S. pombe used as a host, it is preferable to use those having a marker for selecting a transformant. For example, it is preferable to use a host which essentially requires a specific nutritional component for growth due to the lack of certain genes. In a case where a transformant is prepared by transformation using a vector including a target gene sequence, if the lacked gene (complementary auxotrophic marker) is incorporated in advance into the vector, the auxotrophy of the host disappears in the transformant. By the difference in auxotrophy between the host and the transformant, it is possible to make a differentiation between a host and a transformant and to obtain a transformant.

For example, by using S. pombe, which becomes uracil auxotrophic due to the deletion or deactivation of an orotidine phosphate decarboxylase gene (ura4 gene), as a host, performing transformation using a vector having a ura4 gene (complementary auxotrophic marker), and then selecting a transformant in which uracil auxotrophy has disappeared, a transformant into which the vector is incorporated can be obtained. The missing gene that makes the host auxotrophic is not limited to the ura4 gene as long as the gene can be used for selecting a transformant, and may be an isopropylmalate dehydrogenase gene (leu1 gene) or the like.

In addition, the S. pombe in which a group of PDC genes have not undergone deletion or deactivation can be used as a host for manufacturing a transformant. In this case, as a host, it is possible to use a host in which the aforementioned gene (an auxotrophic marker, a protease gene, or the like) other than the PDC genes has undergone deletion or deactivation. By manufacturing a transformant by using the host and then deleting or deactivating some of the genes in a group of PDC genes of the obtained transformant, the transformant according to the present invention can be obtained.

<Method for Introducing Foreign Genes>

As a method for introducing foreign genes into a host by a genetic engineering method, a known method can be used. As a method in which S. pombe is used as a host and structural genes of heterologous proteins are introduced into the host, for example, it is possible to use the methods described in Japanese Unexamined Patent Application, First Publication No. H05-15380, PCT International Publication No. WO95/09914, Japanese Unexamined Patent Application, First Publication No. H10-234375, Japanese Unexamined Patent Application, First Publication No. 2000-262284, Japanese Unexamined Patent Application, First Publication No. 2005-198612, PCT International Publication No. WO2011/021629, and the like.

<Expression Cassette>

An expression cassette is a combination of DNA necessary for expressing a target protein, and includes a structural gene which encodes the target protein and a promoter and a terminator which function in a host. An expression cassette used for manufacturing the transformant according to the present invention includes at least either a PYC gene or an MDH gene as well as a promoter and a terminator which function in S. pombe.

The expression cassette may include any one or more domains among a 5′-untraslated domain and a 3′-untraslated domain. Furthermore, the cassette may include the aforementioned complementary auxotrophic marker. A plurality of foreign genes may be present in a single expression cassette. The number of foreign genes in a single expression cassette is preferably 1 to 8, and more preferably 1 to 5.

In a case where a plurality of foreign genes is included in a single expression cassette, the cassette may include two or more kinds of foreign genes. As the expression cassette, an expression cassette is preferable which includes one or a plurality of PYC genes and MDH genes, a promoter, a terminator, a 5′-untraslated domain, a 3′-untraslated domain, and a complementary auxotrophic marker.

In manufacturing the transformant according to the present invention, the PYC genes and the MDH genes may be introduced into the host by different expression cassettes or by a single expression cassette. As the expression cassette including the PYC genes and the MDH genes, for example, an expression cassette is preferable which includes a promoter, PYC genes, a cleavage sequence, a complementary auxotrophic marker (for example, a Ura4 gene), MDH genes, and a terminator from the 5′ terminal side.

As a gene sequence of the PYC genes or the MDH genes included in the expression cassette, a gene encoded by a wild type may be used as it is. In order to increase the expression amount in S. pombe used as a host, the gene sequence of the wild type may be modified into a codon used at a high frequency in S. pombe.

The promoter and the terminator functioning in S. pombe should be able to function in a transformant and maintain the expression of a protein encoded by foreign genes, even if an acidic condition (pH of equal to or less than 6) is created due to the accumulation of a C4 dicarboxylic acid by the transformant according to the present invention. As the promoter functioning in S. pombe, it is possible to use a promoter (preferably a promoter having high transcription initiation activity) inherent to S. pombe or a promoter (such as a promoter derived from a virus) not being inherent to S. pombe. Herein, two or more kinds of promoter may be present in a vector.

Examples of the promoter inherent to S. pombe include an alcohol dehydrogenase gene promoter, an nmt1 gene promoter involved in the thiamine metabolism, fructose-1,6-bisphosphatase gene promoter involved in the glucose metabolism, an invertase gene promoter involved in the catabolite repression (see PCT International Publication No. WO99/23223), a heat-shock protein gene promoter (see PCT International Publication No. WO2007/26617), and the like.

Examples of the promoter not being inherent to S. pombe include promoters derived from animal cell viruses, described in Japanese Unexamined Patent Application, First Publication No. H05-15380, Japanese Unexamined Patent Application, First Publication No. H07-163373, and Japanese Unexamined Patent Application, First Publication No. H10-234375. As such promoters, an hCMV promoter and an SV40 promoter are preferable.

As the terminator functioning in S. pombe, it is possible to use a terminator inherent to S. pombe or a terminator not being inherent to S. pombe. Herein, two or more kinds of terminator may be present in a vector.

Examples of the terminator include terminators derived from human beings, described in Japanese Unexamined Patent Application, First Publication No. H05-15380, Japanese Unexamined Patent Application, First Publication No. H07-163373, and Japanese Unexamined Patent Application, First Publication No. H10-234375. As such terminators, a terminator of human lipocortin I is preferable.

<Vector>

The transformant according to the present invention has an expression cassette, which includes foreign genes, in a chromosome or has an expression cassette as an extrachromosomal gene. Having the expression cassette in a chromosome means a state where the expression cassette is incorporated into one or more sites in a chromosome of the host cell. Having the expression cassette as an extrachromosomal gene means a state where the transformant has a plasmid including the expression cassette in a cell. The transformant having each expression cassette is obtained by transforming S. pombe as a host by using a vector including each expression cassette.

The vector can be manufactured by incorporating the expression cassette into a vector having a cyclic DNA structure or a linear DNA structure. In a case where a transformant in which the expression cassette is retained as an extrachromosomal gene in a host cell is prepared, the vector is preferably a plasmid including a sequence to be replicated in the host cell, that is, an Autonomously Replicating Sequence (ARS).

In contrast, in a case where a transformant in which the expression cassette is incorporated into a chromosome of a host cell is prepared, the vector is preferably a vector which has a linear DNA structure, does not have ARS, and is introduced into the host cell. For example, the vector may be a vector consisting of linear DNA or a vector having a cyclic DNA structure that has a restriction enzyme recognition sequence for cutting and opening the vector into linear DNA when being introduced into the host. In a case where the vector is a plasmid having ARS, a linear DNA structure can be established by deleting the ARS portion or by deactivating the function of ARS by cleaving the ARS portion, and then the vector can be introduced into a host.

The vector preferably has a marker for selecting a transformant. Examples of the marker include a ura4 gene (complementary auxotrophic marker) and an isopropylmalate dehydrogenase gene (leu1 gene).

Each foreign gene is preferably introduced into a chromosome of S. pombe. By the introduction of the foreign gene into the chromosome, a transformant which is excellently stably maintained in a passage is obtained. Furthermore, a plurality of foreign genes can be introduced into the chromosome. In the transformant according to the present invention, the number of PYC genes incorporated into a chromosome is preferably 1 to 20 and particularly preferably 1 to 8. In addition, the number of MDH genes incorporated into a chromosome of the transformant is preferably 1 to 20 and particularly preferably 1 to 8.

As a method for introducing foreign genes into a chromosome, a known method can be used. For example, by the method described in Japanese Unexamined Patent Application, First Publication No. 2000-262284, a plurality of foreign genes can be introduced into a chromosome. By the same method, a single foreign gene can be introduced into a chromosome. Furthermore, as will be described later, one or a plurality of foreign genes can be introduced into a plurality of sites of a chromosome.

As a method for introducing foreign genes into a chromosome of S. pombe, a method is preferable in which the foreign genes are introduced by a homologous recombination method by using a vector having an expression cassette, which has the foreign genes, and a recombination site.

The recombination site of a vector is a site having a base sequence that can cause homologous recombination with a target site of homologous recombination in a chromosome of S. pombe. The target site is a site into which an expression cassette is incorporated in a chromosome of S. pombe. The target site can be freely set by designing the base sequence of the recombination site of the vector such that the recombination site can cause homologous recombination with the target site.

The base sequence of the recombination site and the base sequence of the target site need to share identity of equal to or higher than 70%. Furthermore, in view of facilitating the occurrence of homologous recombination, the identity shared between the base sequence of the recombination site and the base sequence of the target site is preferably equal to or higher than 90%, and more preferably equal to or higher than 95%. By using the vector having the recombination site described above, an expression cassette can be incorporated into the target site through homologous recombination.

The length (number of bases) of the recombination site is preferably 20 bp to 2,000 bp. If the length of the recombination site is equal to or greater than 20 bp, homologous recombination easily occurs. If the length of the recombination site is equal to or less than 2,000 bp, it is easy to prevent a case where the vector becomes too long and thus the homologous recombination does not easily occur. The length of the recombination site is more preferably equal to or greater than 100 bp, and even more preferably equal to or greater than 200 bp. In addition, the length of the recombination site is more preferably equal to or less than 800 bp, and even more preferably equal to or less than 400 bp.

The vector may have other DNA domains in addition to the aforementioned expression cassette and recombination site. Examples of the DNA domains include a replication initiation domain called “ori” that is necessary for the replication in E. coli and an antibiotic resistance gene (a neomycin resistance gene or the like). These are genes generally required in a case where a vector is constructed using E. coli. Here, it is preferable that the replication initiation domain is removed when the vector is incorporated into a chromosome of a host as will be described later.

In a case where foreign genes are incorporated into a chromosome, the vector preferably has a linear DNA structure when being introduced into a cell of S. pombe. That is, in a case where the vector has a cyclic DNA structure such as plasmid DNA that is generally used, it is preferable that the vector is introduced into the cell of S. pombe after being cut and opened to become linear by a restriction enzyme.

In this case, a position in which the vector having a cyclic DNA structure is cut and opened is in the recombination site. As a result, in each of both ends of the cut and opened vector, the recombination site is partially present, and through homologous recombination, the entirety of the vector is incorporated into a target site of a chromosome.

As long as a linear DNA structure can be established for the vector such that a portion of the recombination site is present in each of both ends thereof, the vector may be constructed by a method other than the method of cutting and opening the vector having a cyclic DNA structure.

As the vector, for example, plasmids derived from E. coli, such as pBR 322, pBR 325, pUC 118, pUC 119, pUC 18, and pUC 19, can be suitably used.

In this case, it is preferable that a replication initiation domain called “ori” necessary for the replication in E. coli is removed from the plasmid vector used at the time of homologous recombination. In this way, when the aforementioned vector is incorporated into a chromosome, the incorporation efficiency can be improved.

A method for constructing a vector, from which the replication initiation domain has been removed, is not particularly limited, but it is preferable to use the method described in Japanese Unexamined Patent Application, First Publication No. 2000-262284. That is, it is preferable to use a method of constructing in advance a precursor vector in which a replication initiation domain is inserted into a cleavage site in a recombination site such that the vector has the linear DNA structure described above and the replication initiation domain is cut off. By this method, a vector from which a replication initiation domain has been removed can be easily obtained.

Furthermore, it is also preferable to use a method in which a precursor vector having an expression cassette and a recombination site is constructed by using the expression vector described in Japanese Unexamined Patent Application, First Publication No. H05-15380, Japanese Unexamined Patent Application, First Publication No. H07-163373, PCT International Publication No. WO96/23890, Japanese Unexamined Patent Application, First Publication No. H10-234375, and the like or using the construction method thereof, and a replication initiation domain is removed from the precursor vector by a general genetic engineering method so as to obtain a vector used for homologous recombination.

<Target Site>

The target site into which the vector is incorporated may be present in only one site or two or more sites in a chromosome of S. pombe. In a case where two or more target sites are present, the vector is incorporated into two or more sites of a chromosome of S. pombe. In a case where a plurality of foreign genes is included in a single expression cassette, the plurality of foreign genes can be incorporated into one target site.

In addition, by using two or more kinds of vector having a recombination site corresponding to each of the target sites, the expression cassette can be incorporated into two or more kinds of target site. By this method, a plurality of foreign genes can be incorporated into a chromosome of S. pombe. As a result, an expression amount of PYC or MDH encoded by the foreign genes can be increased, and the productivity of a C4 dicarboxylic acid can be improved.

For example, by incorporating an expression cassette including a PYC gene into a vector having a first target site, incorporating an expression cassette including an MDH gene into a vector having a second target site, performing transformation by using the vectors and S. pombe, in which some of the genes in a group of PDC genes have undergone deletion or deactivation, as a host, and then deleting or deactivating mae2 genes of the obtained transformant, the transformant according to the present invention is obtained.

In a case where an expression cassette is incorporated into one target site, for example, it is possible to use the target site shown in the method described in Japanese Unexamined Patent Application, First Publication No. 2000-262284. By using two or more kinds of vector having different incorporation sites, the vectors can be incorporated into different target sites respectively. However, the above method is complicated for incorporating vectors into two or more sites of a chromosome.

As long as a plurality of portions, which is present in a chromosome and has base sequences substantially the same as each other, can be used as target sites, and a vector can be incorporated into each of the plurality of target sites, the vector can be incorporated into two or more sites in the chromosome by using one kind of vector.

The base sequences substantially the same as each other mean that the sequences share identity of equal to or higher than 90%. The identity shared between the target sites is preferably equal to or higher than 95%. The length of each of the base sequences substantially the same as each other is a length including the recombination site of the aforementioned vector, which is preferably equal to or greater than 1,000 bp.

In a case where foreign genes are incorporated into a plurality of target sites in a dispersed state, even if the same number of foreign genes are incorporated into the target sites, a phenomenon in which the foreign genes are broken away all at once from a chromosome when the transformant grows occurs less than in a case where a plurality of foreign genes is incorporated into a single target site. Therefore, the transformant is more stably maintained in passage.

As the plurality of target sites present in a chromosome, a transposon gene Tf2 is preferable. Tf2 is a transposon gene present in a total of 13 sites in each triple-strand (monoploid) chromosome of S. pombe. The length (number of bases) thereof is known to be about 4,900 bp, and the base sequence identity shared between the genes thereof is known to be 99.7% (see the following documents).

Nathan J. Bowen et al, “Retrotransposons and Their Recognition of pol II Promoters: A Comprehensive Survey of the Transposable Elements from the Complete Genome Sequence of Schizosaccharomyces pombe”, Genome Res. 2003 13: 1984-1997

It is possible to incorporate a vector into only one of the Tf2's present in 13 sites in a chromosome. In this case, by incorporating a vector having two or more foreign genes, a transformant having two or more foreign genes can be obtained. Furthermore, by incorporating a vector into two or more Tf2's, a transformant having two or more foreign genes can be obtained. In this case, by incorporating the vector having two or more foreign genes, a transformant having more foreign genes can be obtained. If a vector is incorporated into all of the 13 Tf2's, too much burden may be imposed on the survival or growth of the transformant. Therefore, the vector is preferably incorporated into 8 or less of the 13 Tf2's, and more preferably incorporated into 5 or less Tf2's.

<Transformation Method>

As a transformation method, any of known transformation methods can be used. Examples of the transformation method include the methods known in the related art, such as a lithium acetate method, an electroporation method, a spheroplast method, and a glass bead method, and the method described in Japanese Unexamined Patent Application, First Publication No. 2005-198612. Furthermore, commercially available yeast transformation kits may be used.

As a method for transforming the S. pombe host by a homologous recombination method, a known homologous recombination method can be used. As a transformation method at the time of manufacturing the transformant according to the present invention, a method is preferable wherein S. pombe in which some of the genes in a group of PDC genes described above have undergone deletion or deactivation is used as a host, and an expression cassette is incorporated into a chromosome of S. pombe through homologous recombination by using the vector described above.

At the time of manufacturing the transformant, generally, after homologous recombination is performed, the obtained transformant is selected. As the selection method, for example, the following method can be used. By using a medium which makes it possible to select transformants by using the aforementioned auxotrophic marker, screening is carried out, thereby selecting a plurality of transformants from the obtained colony.

Then, each of the transformants is individually subjected to liquid culture. Thereafter, an expression amount of a heterologous protein in each culture solution is investigated, and transformants showing a greater expression amount of the heterologous protein are selected. Through a pulse field gel electrophoresis method, genomic analysis is performed on the selected transformants, and in this way, the number of vectors or expression cassettes incorporated into a chromosome is investigated.

The number of vectors incorporated into a chromosome can be adjusted to some extent by adjusting the incorporation conditions or the like. It is considered that the incorporation efficiency or the number of vectors incorporated may vary with the size (number of bases) or structure of the vector.

The transformant according to the present invention has malic acid producibility that is better than ever. A malic acid production rate of the transformant is preferably equal to or higher than 2.0 g/(L·h), more preferably equal to or higher than 5.0 g/(L·h), even more preferably equal to or higher than 10 g/(L·h), and particularly preferably 15 g/(L·h) to 30 g/(L·h).

[Method for Manufacturing C4 Dicarboxylic Acid]

A method for manufacturing a C4 dicarboxylic acid according to the present invention includes culturing the transformant according to the present invention in a culture solution and obtaining a C4 dicarboxylic acid from the culture solution.

By culturing of the transformant according to the present invention in a sugar-containing culture solution, oxaloacetic acid is produced from pyruvic acid obtained from the sugar by a glycolytic system with the aid of PYC or produced from phosphoenolpyruvic acid with the aid of PCK. From the produced oxaloacetic cid, malic acid is produced with the aid of MDH, and from the produced malic acid, other C4 dicarboxylic acids are produced as well.

Although the C4 dicarboxylic acids including the produced malic acid are accumulated in the microbial cell, some of them are released to a culture supernatant due to Mae1 (C4 dicarboxylic acid transporter). By obtaining C4 dicarboxylic acids from the cultured transformant or the culture supernatant, it is possible to manufacture C4 dicarboxylic acids. Examples of C4 dicarboxylic acids manufactured from the transformant include oxaloacetic acid, malic acid, fumaric acid, succinic acid, and the like, and among these, oxaloacetic acid or malic acid is preferable.

As the culture solution used for manufacturing a C4 dicarboxylic acid, a known sugar-containing culture medium for yeast can be used. Furthermore, the culture medium should contain a nitrogen source, inorganic salts, and the like that S. pombe can utilize and should enable S. pombe to be efficiently cultured. As the culture solution, a natural medium or a synthetic medium may be used.

Examples of the sugar as a carbon source include sugars such as glucose, fructose, sucrose, and maltose. Examples of the nitrogen source include ammonia, an ammonium salt of an inorganic or organic acid, such as ammonium chloride or ammonium acetate, peptone, casamino acid, yeast extract, and the like. Examples of the inorganic salts include magnesium phosphate, magnesium sulfate, sodium chloride, and the like. It is also possible to further add a fermentation accelerating factor such as proteolipid.

In the method for manufacturing a C4 dicarboxylic acid according to the present invention, it is preferable to use a culture solution particularly containing glucose or sucrose as sugar. A concentration of glucose or sucrose in the culture solution (100% by mass) at the initial stage of culture is preferably equal to or greater than 1% by mass, more preferably 1% by mass to 50% by mass, and even more preferably 2% by mass to 16% by mass. After the glucose concentration or the sucrose concentration is reduced due to culture, it is preferable to continue the culture by adding glucose as necessary. At the final stage of culture, the glucose concentration or the like may become equal to or less than 1% by mass.

In a case where continuous culture is performed by circulating the culture solution in a state of separating a C4 dicarboxylic acid, it is preferable to maintain the glucose concentration or the like. If the glucose concentration is set to be equal to or greater than 2% by mass, the productivity of a C4 dicarboxylic acid is further improved. Furthermore, if the concentration of glucose or sucrose in the culture solution is set to be equal to or less than 16% by mass, the production efficiency of a C4 dicarboxylic acid is further improved.

In order to improve the productivity of manufacturing of a C4 dicarboxylic acid, it is preferable to perform high-density culture. During the high-density culture, the initial microbial cell concentration of the transformant in the culture solution, expressed in terms of a weight of dry microbial cells, is preferably set to be 0.1 g/L to 100 g/L. The initial microbial cell concentration of the transformant in the culture solution, expressed in terms of a weight of dry microbial cells, is more preferably set to be 20 g/L to 60 g/L. If the initial microbial cell concentration is set to be high, high productivity can be achieved within a short period of time. Furthermore, if the initial microbial cell concentration is too high, a problem such as the aggregation of microbial cells or the reduction of purification efficiency may occur.

The microbial cell concentration described in examples and the like, which will be described later, is a value converted from an absorbance (OD₆₆₀) of light having a wavelength of 660 nm measured by a visible-ultraviolet spectrometer V550 manufactured by JASCO Corporation. The value of 1 that equals OD₆₆₀ at 660 nm corresponds to a dry weight of 0.2 g and a wet weight of 0.8 g of fission yeast.

For the culture, a known yeast culture method can be used. For example, shake culture or stirring culture can be performed.

The culture temperature is preferably 23° C. to 37° C., and the culture time can be appropriately determined.

The culture may be batch culture or continuous culture. For example, after culture is performed by batch culture, by separating the microbial cells from the culture solution, a culture solution containing a C4 dicarboxylic acid can be obtained. In addition, examples of the continuous culture method include a method of continuously performing culture by repeating a process of taking out a portion of the culture solution from a culture tank in which culture is being performed, separating a C4 dicarboxylic acid from the taken culture solution and recovering the culture supernatant, adding glucose or a new culture solution to the culture supernatant, and returning the culture supernatant to the culture tank. By performing the continuous culture, the productivity of a C4 dicarboxylic acid is further improved.

The transformant according to the present invention has particularly excellent acid resistance. Therefore, even at a low pH (about pH 2 to 4) resulting from the accumulation of a C4 dicarboxylic acid, the transformant can produce a C4 dicarboxylic acid without being subjected to neutralization. Accordingly, even after the pH of the culture solution becomes equal to or less than 3.5, it is possible to manufacture a C4 dicarboxylic acid by continuous culture in which culture is continued. The pH of the last stage of culture and the pH during the continuous culture is preferably equal to or less than 3.5 and particularly preferably 2.3 to 3.5. In some cases, culture may be finished before the pH of the culture solution becomes equal to or less than 3.5.

A C4 dicarboxylic acid can be obtained from the culture solution by a known method. Examples of the method include a method in which microbial cells are separated by centrifugation from the culture solution after the end of the culture, and a C4 dicarboxylic acid is extracted using diethyl ether or ethyl acetate after the pH becomes equal to or less than 1; a method in which the culture solution is absorbed onto an ion exchange resin and washed, and then a C4 dicarboxylic acid is eluted; a method in which impurities are removed using activated carbon; a method in which the culture solution is reacted with alcohol in the presence of an acid catalyst and then subjected to distillation; and a method in which a C4 dicarboxylic acid is separated using a separation membrane.

Furthermore, in some cases, by neutralizing a C4 dicarboxylic acid in the culture solution and then separating a C4 dicarboxylic acid salt from the culture solution, a C4 dicarboxylic acid can be obtained. For example, by a method of converting a C4 dicarboxylic acid in the culture solution into a calcium salt or a lithium salt and crystallizing the neutralized salt, a C4 dicarboxylic acid can also be obtained.

The method for manufacturing a C4 dicarboxylic acid according to the present invention described above makes it possible to manufacture a C4 dicarboxylic acid with high productivity in a simple manner by using a transformant that uses S. pombe as a host. According to the manufacturing method, a production rate of a C4 dicarboxylic acid easily becomes equal to or higher than 5 g/L/h, and in some cases, the production rate of a C4 dicarboxylic acid becomes equal to or higher than 15 g/L/h. The method for manufacturing a C4 dicarboxylic acid according to the present invention is also suitable for high-density culture that is performed in the presence of high-concentration glucose by using a high-concentration transformant.

### EXAMPLES

Hereinafter, the present invention will be specifically described by showing examples and comparative examples, but the present invention is not limited to the following description. In the present examples, unless otherwise specified, “%” means “% by mass”.

<Preparation of PDC2 Gene Deletion Strain of S. pombe>

A uracil auxotrophic ARC019 strain of S. pombe (genotype: h⁻ leu1-32, ura4-D18, Ade6-M216) (Strain name: JY741, NBRPID: FY7512) was transformed according to a Latour method (described in the journal of Nucleic Acids Research, 2006, Vol. 34, p. e11, PCT International Publication No. WO2007/063919), thereby preparing a deletion strain (IGF836 strain) from which PDC2 genes (strain name: SPAC1F8. 07c) were deleted.

For preparing a deletion fragment, total genomic DNA prepared from an ARC032 strain of S. pombe (genotype: h⁻) (see PCT International Publication No. WO2007/015470) by using DNeasy (manufactured by QUIAGEN) was used as a template, and 8 kinds of synthetic oligo DNA (manufactured by Operon Biotechnologies) having the base sequences shown in Table 1 were used.

TABLE 1
Oligo DNA for preparing pdc2 deletion fragment

| Oligo | | |
|---|---|---|
| DNA | Base sequence | SEQ ID NO: |
| UF | 5′-CTCTCCAGCTCCATCCATAAG-3′ | 24 |
| UR | 5′-GACACAACTTCCTACCAAAAAGCCTTTCT | 25 |
| | GCCCATGTTTTCTGTC-3′ | |
| OF | 5′-GCTTTTTGGTAGGAAGTTGTGTC-3′ | 26 |
| OR | 5′-AGTGGGATTTGTAGCTAAGCTGT | 27 |
| | ATCCATTTCAGCCGTTTGTG-3′ | |
| DF | 5′-AAGTTTCGTCAATATCACAAGCT | 28 |
| | GACAGAAAACATGGGCAGAAAG-3′ | |
| DR | 5′-GTTCCTTAGAAAAAGCAACTTTGG-3′ | 29 |
| FF | 5′-CATAAGCTTGCCACCACTTC-3′ | 30 |
| FR | 5′-GAAAAAGCAACTTTGGTATTCTGC-3′ | 31 |

Specifically, by a PCR method using KOD-Dash (manufactured by TOYOBO CO., LTD.), a UP domain was prepared using UF and UR, an OL domain was prepared using OF and OR, and a DN domain was prepared using DF and DR. Then, by using these as templates, a full-length deletion fragment was prepared by the same PCR method using FF and FR respectively. At the time of preparing the full-length deletion fragment, 2 kinds of synthetic oligo DNA (manufactured by Operon Biotechnologies) having the base sequences shown in Table 2 were used, and the total genomic DNA prepared from the ARC032 strain in the same manner as above was used as a template. Furthermore, a fragment of a domain of a uracil auxotrophic marker ura4 of S. pombe (strain name listed in GeneDB: SPCC330.05c, orotidine-5′-phosphate decarboxylase gene) prepared by the same PCR method was also used in combination as a template.

TABLE 2
Oligo DNA for preparing ura4 fragment

| Oligo DNA | Base sequence | SEQ ID NO: |
|---|---|---|
| F | 5′-AGCTTAGCTACAAATCCCACT-3′ | 32 |
| R | 5′-AGCTTGTGATATTGACGAAACTT-3′ | 33 |

By using a DNA fragment obtained by treating a pSHh vector with a restriction enzyme PmaCI, the obtained PDC2 gene deletion strain of S. pombe (IGF836 strain, h⁻leu1-32 ura4-D18 ade6-M216 pdc2-D23) was transformed, thereby obtaining an ASP4590 strain in which uracil auxotrophy and adenine auxotrophy were restored.

### Example 2

<Preparation of Foreign Gene Introduction Strain>

Transformants of pombe were prepared (Table 24) into which PYC derived from Aspergillus niger (AniPYC) (SEQ ID NO: 1), PYC derived from Brevibacillus brevis (BbrPYC) (SEQ ID NO: 2), PYC derived from Debaryomyces hansenii (DhaPYC) (SEQ ID NO: 3), PYC derived from Kluyveromyces lactis (KlaPYC) (SEQ ID NO: 4), PYC derived from Lachancea thermotolerans (LthPYC) (SEQ ID NO: 5), PYC derived from Lodderomyces elongisporus (LelPYC) (SEQ ID NO: 6), PYC derived from Saccharomyces cerevisiae (ScePYC) (SEQ ID NO: 7), PYC derived from Candida orthopsilosis (CorPYC) (SEQ ID NO: 8), PYC derived from Candida tropicalis (CtrPYC) (SEQ ID NO: 9), PYC derived from Naumovozyma castellii (NcaPYC) (SEQ ID NO: 10), PYC derived from Naumovozyma dairenensis (NdaPYC) (SEQ ID NO: 11), PYC derived from Saccharomyces kudriavzevii (SkuPYC) (SEQ ID NO: 12), PYC derived from S. pombe (SpoPYC) (SEQ ID NO: 13), PYC derived from Tetrapisispora blattae (TblPYC) (SEQ ID NO: 14), PYC derived from Torulaspora delbrueckii (TdePYC) (SEQ ID NO: 15), PYC derived from Zygosaccharomyces rouxii (ZroPYC) (SEQ ID NO: 16), MDH derived from Archaeoglobus fulgidus (AfuMDH) (SEQ ID NO: 17), MDH derived from Congregibacter litoralis (CliMDH) (SEQ ID NO: 18), MDH derived from Delftia acidovorans (DacMDH) (SEQ ID NO: 19), MDH derived from Halomonas elongata (HelMDH) (SEQ ID NO: 20), or MDH derived from Shewanella putrefaciens (SpuMDH) (SEQ ID NO: 21) was introduced.

Specifically, The IGF836 strain (gene deletion strain of S. pombe) prepared in Example 1 was transformed according to the method of Bahler et al. (the journal of Yeast, 1998, Vol. 14, pp. 943-951) by using a digest of a restriction enzyme BsiWI of a monodentate integrative recombinant vector pSLh-AniPYC retaining an AniPYC gene expression cassette, a monodentate integrative recombinant vector pSLh-BbrPYC retaining a BbrPYC gene expression cassette, a monodentate integrative recombinant vector pSLh-DhaPYC retaining a DhaPYC gene expression cassette, a monodentate integrative recombinant vector pSLh-KlaPYC retaining a KlaPYC gene expression cassette, a monodentate integrative recombinant vector pSLh-LthPYC retaining an LthPYC gene expression cassette, a monodentate integrative recombinant vector pSLh-LelPYC retaining an LelPYC gene expression cassette, a monodentate integrative recombinant vector pSLh-ScePYC retaining an ScePYC gene expression cassette, a monodentate integrative recombinant vector pSLh-CorPYC retaining a CorPYC gene expression cassette, a monodentate integrative recombinant vector pSLh-CtrPYC retaining a CtrPYC gene expression cassette, a monodentate integrative recombinant vector pSLh-NcaPYC retaining an NcaPYC gene expression cassette, a monodentate integrative recombinant vector pSLh-NdaPYC retaining an NdaPYC gene expression cassette, a monodentate integrative recombinant vector pSLh-SkuPYC retaining an SkuPYC gene expression cassette, a monodentate integrative recombinant vector pSLh-SpoPYC retaining an SpoPYC gene expression cassette, a monodentate integrative recombinant vector pSLh-TblPYC retaining a TblPYC gene expression cassette, a monodentate integrative recombinant vector pSLh-TdePYC retaining a TdePYC gene expression cassette, a monodentate integrative recombinant vector pSLh-ZroPYC retaining a ZroPYC gene expression cassette, a monodentate integrative recombinant vector pSMh-AfuMDH retaining an AfuMDH gene expression cassette, a monodentate integrative recombinant vector pSMh-CliMDH retaining a CliMDH gene expression cassette, a monodentate integrative recombinant vector pSMh-DacMDH retaining a DacMDH gene expression cassette, a monodentate integrative recombinant vector pSMh-HelMDH retaining an HelMDH gene expression cassette, or a monodentate integrative recombinant vector pSMh-SpuMDH retaining an SpuMDH gene expression cassette.

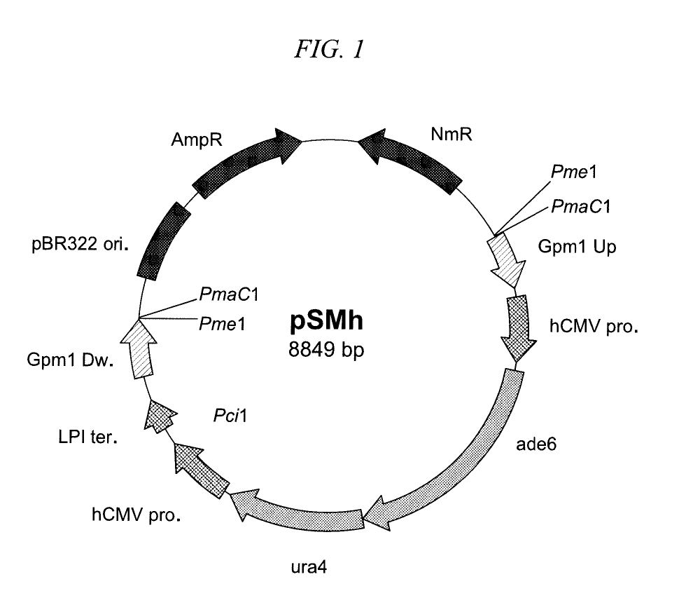

The monodentate integrative recombinant vector pSMh can be prepared through the following process. First, a fragment obtained by digestion of a DNA fragment (Fr. 1), which was prepared by total synthesis of DNA and had a base sequence represented by SEQ ID NO: 34, with a restriction enzyme BsiWI and a DNA fragment, which was obtained by the digestion of a pSE vector with a restriction enzyme BsiWI and the double digestion of the obtained digest with restriction enzymes KpnI and SnaBI, were subjected to ligation, thereby preparing pSMh (8,849 bp, FIG. 1) having a base sequence (5′→3′, cyclic) represented by SEQ ID NO: 35.

pSLh-AniPYC was prepared through the following process. First, by using total genomic DNA prepared by DNeasy (manufactured by QUIAGEN) from the culture of Aspergillus niger as a template and using 2 kinds of synthetic oligo DNA (AniPYC-F and AniPYC-R manufactured by Operon Biotechnologies) described in Table 3, an ORF fragment of an AniPYC gene was obtained by a PCR method using KOD-Dash (manufactured by TOYOBO CO., LTD.). The ORF fragment encoded AniPYC (SEQ ID NO: 1).

TABLE 3

| | Sequence | SEQ ID NO: |
|---|---|---|
| AniPYC-F | 5′-GACACTTTTTCAAACATGGCTGCTCCCCGCC-3′ | 36 |
| AniPYC-R | 5′-ATCATCCTTGTAATCGGCCTTGACGATCTTGCAGAC-3′ | 37 |
| AniPYC | MAAPRQPEEAVDDTEFIDDHHDQHRDSVHTRLRANSAIMQFQKILV | 1 |
| | ANRGEIPIRIFRTAHELSLQTVAVYSHEDRLSMHRQKADEAYMIGK | |
| | RGQYTPVGAYLAIDEIVKIALEHGVHLIHPGYGFLSENAEFARKVE | |
| | QSGMVFVGPTPQTIESLGDKVSARQLAIRCDVPVVPGTPGPVERYE | |
| | EVKAFTDTYGFPIIIKAAFGGGGRGMRVVRDQAELRDSFERATSEA | |
| | RSAFGNGTVFVERFLDRPKHIEVQLLGDNHGNVVHLFERDCSVQRR | |
| | HQKVVEIAPAKDLPADVRDRILADAVKLAKSVNYRNAGTAEFLVDQ | |
| | QNRYYFIEINPRIQVEHTITEEITGIDIVAAQIQIAAGATLEQLGL | |
| | TQDRISTRGFAIQCRITTEDPSKGFSPDTGKIEVYRSAGGNGVRLD | |
| | GGNGFAGAIITPHYDSMLVKCTCRGSTYEIARRKVVRALVEFRIRG | |
| | VKTNIPFLTSLLSHPVFVDGTCWTTFIDDTPELFALVGSQNRAQKL | |
| | LAYLGDVAVNGSSIKGQIGEPKLKGDIIKPVLHDAAGKPLDVSVPA | |
| | TKGWKQILDSEGPEAFARAVRANKGCLIMDTTWRDAHQSLLATRVR | |
| | TIDLLNIAHETSHALANAYSLECWGGATFDVAMRFLYEDPWDRLRK | |
| | LRKAVPNIPFQMLLRGANGVAYSSLPDNAIYHFCKQAKKCGVDIFR | |
| | VFDALNDVDQLEVGIKAVHAAEGVVEATICYSGDMLNPSKKYNLPY | |
| | YLDLVDKVVQFKPHVLGIKDMAGVLKPQAARLLIGSIRERYPDLPI | |
| | HVHTHDSAGTGVASMIACAQAGADAVDAATDSLSGMTSQPSIGAIL | |
| | ASLEGTEHDPGLNSAQVRALDTYWAQLRLLYSPFEAGLTGPDPEVY | |
| | EHEIPGGQLTNLIFQASQLGLGQQWAETKKAYESANDLLGDVVKVT | |
| | PTSKVVGDLAQFMVSNKLTAEDVIARAGELDFPGSVLEFLEGLMGQ | |
| | PYGGFPEPLRSRALRDRRKLDKRPGLYLEPLDLAKIKSQIRENYGA | |
| | ATEYDVASYAMYPKVFEDYKKFVAKFGDLSVLPTRYFLAKPEIGEE | |
| | FHVELEKGKVLILKLLAIGPLSEQTGQREVFYEVNGEVRQVSVDDK | |
| | KASVENTARPKAELGDSSQVGAPMSGVVVEIRVHDGLEVKKGDPIA | |
| | VLSAMKMEMVISAPHSGKVSSLLVKEGDSVDGQDLVCKIVKA* | |

By using an In-Fusion (registered trademark) HD Cloning Kit (manufactured by Clontech Laboratories, Inc.), the obtained amplified fragment was incorporated into pSLh, thereby preparing pSLh-AniPYC. The In-Fusion method was performed according to the manual included in the kit. That is, the obtained PCR product was purified using a spin column, added to an In-Fusion reaction solution together with pSLh, and reacted for 15 minutes at 50° C.

By the same method as described above, a monodentate integrative recombinant vector pSLh-BbrPYC, a monodentate integrative recombinant vector pSLh-DhaPYC, a monodentate integrative recombinant vector pSLh-KlaPYC, a monodentate integrative recombinant vector pSLh-LthPYC, a monodentate integrative recombinant vector pSLh-LelPYC, a monodentate integrative recombinant vector pSLh-ScePYC, a monodentate integrative recombinant vector pSLh-CorPYC, a monodentate integrative recombinant vector pSLh-CtrPYC, a monodentate integrative recombinant vector pSLh-NcaPYC, a monodentate integrative recombinant vector pSLh-NdaPYC, a monodentate integrative recombinant vector pSLh-SkuPYC, a monodentate integrative recombinant vector pSLh-SpoPYC, a monodentate integrative recombinant vector pSLh-TblPYC, a monodentate integrative recombinant vector pSLh-TdePYC, a monodentate integrative recombinant vector pSLh-ZroPYC, a monodentate integrative recombinant vector pSMh-AfuMDH, a monodentate integrative recombinant vector pSMh-CliMDH, a monodentate integrative recombinant vector pSMh-DacMDH, a monodentate integrative recombinant vector pSMh-HelMDH, and a monodentate integrative recombinant vector pSMh-SpuMDH were prepared. Primer sets using the respective vectors will be listed below.

The ASP4590 strain was transformed using the monodentate integrative recombinant vector pSLh-ScePYC and the monodentate integrative recombinant vector pSMh-DacMDH, thereby obtaining an ASP4491 strain into which an ScePYC gene and a DacMDH gene were introduced.

TABLE 4

| | Sequence | SEQ ID NO: |
|---|---|---|
| BbrPYC-F | 5′-GACACTTTTTCAAACATGAAAAAGAGAAAAATCAATCGCCTAC | 38 |
| | TG-3′ | |
| BbrPYC-R | 5′-ATCATCCTTGTAATCCTCCATTTCAATGAGCAGATCACCA-3′ | 39 |
| BbrPYC | MKKRKINRLLVANRGEIAIRIFRAATELGIRTVAVYSEQDNVSTHR | 2 |
| | FKADESYLVGAGKGPIEAYLDIESIIEIAKRNDIDAVHPGYGFLAE | |
| | NAEFAKRCQEEGIIFIGPSPALIDKFGDKVEARRLAIEAEIPVIPG | |
| | TPEPIETLQEALLFAKEYGYPIIIKGVSGGGGRGMRIVRSQEGLQD | |
| | SLDRARSEARSSFGNAKVYLERYLEQPKHIEVQILGDNHGNIVYLY | |
| | ERDCSVQRRHQKVVEVAPSLSLDDKLREDICQAALTLMKKAGYSNA | |
| | GTVEFLLTPDKRFYFIEVNPRIQVEHTITELITGIDIVQSQIRVAE | |
| | GHALSDEEIGIRAQSCIQMSGFAIQCRVTTEDAENNFLPDAGRLSA | |
| | WRSGGGFGVRLDGGNGYPGAIITFFYDSLIVKISTYGASFDQAARK | |
| | MLRTLREFRIRGVKTNLPFLENVVTHPDFLSGNYDTSFIDTKPELF | |
| | IFPGRQDRGTKLLSYIGDTIVNGYPGLPKSEKKPHFDSPRIPRTPF | |
| | TQPYPDGTKQILDKEGADGLVRWIQAQKQVLVTDTTFRDAHQSLFA | |
| | TRVRTYDLASIAEATGKLGSGLFSLEMWGGATFDTTKRFLQESPWE | |
| | RLQILRGRIPNVLFQMLLRGANGVGYTNYPDNAIHAFVKASAENGI | |
| | DVFRIFDSLNWLPGMQTAIEAVRESGKVAEAAICYTGDILDPTKTK | |
| | YTLAYYVNLAKELEKAGAHILAIKDMAGLLKPYAAYSLVSALKQEI | |
| | SIPIHLHTHDTSGNAGAMLLKAIEAGVDIVDACVSSMSGLTSQPSL | |
| | NGLIASLAHTERETGLSLESFNKLSDYWEDVRPYYQGFESGMKASN | |
| | TEVYVHEMPGGQYTNLEQQAKAVGLEGRWDEVKKMYAVVNQMCGDI | |
| | VKVTPSSKVVGDMALFMVQNNLNEENIWEKGTRLDFPDSVIQFFQG | |
| | YLGQPPGGFPKKLQELVLKGRDAFTARPGELLAPIDFTQVAAELEA | |
| | KIGREPSHLDVLSYIMYPQVYLQFEQRLKEYGDLSVLNTGTFFYGL | |
| | RPGEETAITIERGKTLIIKLVAVGELNPDGRRIIYFELNGQPREIF | |
| | IRDQSAKVSELIRRKAEAQNPAKLGASMPGKVLKVLVAEGDKVRKG | |
| | EHLLVSEAMKMETTIQAPLDGKIKAVYVKAGEAIQTGDLLIEME* | |

TABLE 5

| | Sequence | SEQ ID NO: |
|---|---|---|
| DhaPYC-F | 5′-GACACTTTTCAAACATGAGTGACGGTAATGAACATAAGATCA | 40 |
| | A-3′ | |
| DhaPYC-R | 5′-ATCATCCTTGTAATCTTTAACAACACTTGCAATCAAATCGTTA | 41 |
| | GC-3′ | |
| DhaPYC | MSDGNEHKINQMRRASTVMGPMNKIIVANRGEIPIRIFRTAHELSM | 3 |
| | QTVAIFSHEDRLSMHRLKADESYVIGKKGQFSPVQAYLQIDEIINI | |
| | AKEHGVNMIHPGYGFLSENSEFARKVEEAGISWIGPTHKTIDAVGD | |
| | KVSARTLAIQNGVPVVPGTPGPIADVEEAVKFVEKHGLPVIIKAAF | |
| | GGGGRGMRVVREGDSVGEAFERAVSEAKTSFGNGTCFIERFLDKPK | |
| | HIEVQLLADNYGNVIHLFERDCSVQRRHQKVVEVAPAKTLTHSVRD | |
| | AILTDAVKLARSANYRNAGTAEFLVDEQDRHYFIEINPRIQVEHTI | |
| | TEEITGVDIPAAQIQIAAGASLKQLGLLQDKITTRGFAIQCRITTE | |
| | DPSKNFQPDTGKIEVYRSAGGNGVRLDGGNGFAGSIISPHYDSMLV | |
| | KCSCSGSTYEIVRRKMLRALIEFRIRGVKTNIPFLLAMLTNETFIS | |
| | GDCWTTFIDDTPSLFQMIRSQNRATKILSYLGDLIVNGSSIKGQIG | |
| | EPKLNTEALIPDLHDPKTGIKIEIHNEVAPRGWRQVLLEQGPEKFA | |
| | QKVREFNGTLLMDTTWRDAHQSLLATRVRTIDLLNIAPTTAVALNG | |
| | CFALECWGGATFDVCMRFLYEDPWSRLRQLRKLVPNIPFQMLLRGA | |
| | NGVAYSSLPDNAIDQFVLQAKENGVDIFRVFDALNDLEQLKVGIDA | |
| | VKKAGQVIEATVCYSCDMLKPGKKYNLEYYLGVVDDIVKLGTHFLG | |
| | IKDMAGTLKPKAAKILIGAIRERYPKLPIHVHTHDSAGTGVASMTE | |
| | AAKAGADVVDAASNSMSGMTSQPSINAILASFEGEVESGLQASIVR | |
| | ELDNYWAQMRLLYSCFEADLKGPDPEVYQHEIPGGQLTNLLFQAQQ | |
| | LGLGSKWMLTKEAYKTANKLLGDIVKVTPTSKVVGDLAQFMVSNNL | |
| | SEEDVTKLASELDFPSSVLDFMEGLMGTPYGGFPEPLRTNILGTKR | |
| | VKLNNRPGLNLPPIDFAAVKEELVSRYGSNINECDIASYVMYPKVY | |
| | EDFRELSEKYGDLSVLPTRFFLKACNINEEIVVDIEKGKTLIIKLL | |
| | AIGEISQQTGTREVFFELNGEMRSVTVDDKTVSIETKTRPKATSPN | |
| | EVGAPMAGVVVEIRSKVGNEVKKGDPIAVLSAMKMEMVISSPVSGK | |
| | IGEILIKEGDSVDANDLIASVVK* | |

TABLE 6

| | Sequence | SEQ ID NO: |
|---|---|---|
| KlaPYC-F | 5′-GACACTITTTCAAACATGTCATCTCAACTAGCTGGATTGC- | 42 |
| KlaPYC-R | 5′-ATCATCCTTGTAATCTTCCTTAGCTGGAGCTTCTTCC-3′ | 43 |
| KlaPYC | MSSQLAGLRDNSNLMGEKNKLLVANRGEIPIRIFRTAHELSMKTVA | 4 |
| | IYSHEDRLSMHRLKADEAYVIGEQGKYTPVGAYLAIDEIINIAKSH | |
| | GVNFIHPGYGFLSENSEFAEKVAASGITWVGPPAAVIDSVGDKVSA | |
| | RNLAEKADVPVVPGTPGPIETVEEAQAFVDKYGFPVIIKAAFGGGG | |
| | RGMRVVREGDDISDAPQRAKSEAITAFGNGTCFIERFLDKPKHIEV | |
| | QLLADNYGNVVHLFERDCSVQRRHQKVVEVAPAKTLPEEVRDAILT | |
| | DAVKLAKEAGYRNAGTAEFLVDNQNRHYFIEINPRIQVEHTITEEI | |
| | TGIGIVAAQIQIAAGATLEQLGLMQDKITTRGFAIQCRITTEDPSK | |
| | NFQPDTGRIDVYRSAGGNGVRLDGGNAFAGSVISPHYDSMLVKCSC | |
| | SGSTYEIVRRKMLRALIEFRIRGVKTNIPFLLTLLTHPVFKSGEYW | |
| | TTFIDDTPQLFEMVSSQNRAQKLLHYLADLAVNGSSIKGQIGLPKL | |
| | TTHPTIPHLHNEDGSLVDVSAKPPAGWRDVLLDYGPEEFAKQVRKF | |
| | NGTLLMDTTWRDAHQSLLATRVRTYDLAAIAPTTAHAMSGAFALEC | |
| | WGGATFDVSMRFLHEDPWERLRTLRKLVPNIPFQMLLRGANGVAYS | |
| | SLPDNAIDHFVKQAKDNGVDIFRVFDALNDLEQLKVGVDAVKKAGG | |
| | VVEATICYSGDMLAPGKKYNLDYYLDITEKIVNSGTHILGIKDMAG | |
| | TLKPSAARLLIGSIRAKYPELPIHVHTMDSAGTGVASMAACALSGA | |
| | DVVDVATNSMSGLTSQPSINALLAALDGEIDNNVNVGYVRELDAYW | |
| | AEMRLLYSCFEADLKGPDPEVYVHEIPGGQLTNLLFQAQQLGLGEK | |
| | WAETKRAYREANLLLGDLVKVTPTSKVVGDLAQFMVTNKLTSDDVR | |
| | KLASSLDFPDSVMDFFEGLIGQPYGGPPEPLATDVLRNKRRKLTQR | |
| | PGLELAPFELEKIKEDLSTRFSDIDECDVASYNMYPKVYEDFRKIK | |
| | EKYGDLSVLPTKNFLSPPVIGEEIVVTIEQGKTLIIKPQAIGDLNK | |
| | ETGIREVYFELNGELRKVSVADRSQKVETVSKPKADAHDPFQIGSP | |
| | MAGVVVEVKVHKGSLIAKGQPVAVLSAMKMEMVISSPADGQVKEVL | |
| | VKDGENVDASDLLVVLEEAPAKE* | |

TABLE 7

| | Sequence | SEQ ID NO: |
|---|---|---|
| LthPYC-F | 5′-GACACTTTTTCAAACATGTCCAAACTGGCAGGACTG-3′ | 44 |
| LthPYC-R | 5′-ATCATCCTTGTAATCTTCTTTTGGTGGGACAGATTCCTC-3′ | 45 |
| LthPYC | MSKLAGLRDSSNLLGEKNKLLVANRGEIPIRIFRSAHELSMKTVAI | 5 |
| | YSHEDRLSMHKLKADEAYMIGKEGKYTPVGAYLAIDEILKIAKEHN | |
| | VDFIHPGYGFLSENSEFAQKVEAAGITWIGPSAEVIESVGDKVSAR | |
| | NLAAKADVPVVPGTPGPIDSVEEAQAFVEEYGYPVIIKAAYGGGGR | |
| | GMRVVREGDDIADAFQRATSEAKTAFGNGTCFIERFLNKPKHIEVQ | |
| | LLADGYGNVVHLFERDCSVQRRHQKVVEVAPAKTLPKDVRDAILTD | |
| | AVKLAKVAGYRNAGTAEFLVDCQNRHYFIEINPRIQVEHTITEEIT | |
| | GIDIVAAQIQIAAGASLQELGLLQDKIITRGFAIQCRITTEDPAKN | |
| | FQPDTGRIEVYRSAGGNGVRLDGGNAYAGSIISPHYDSMLVHCSCS | |
| | GSTYEIVRRKMLRALIEFRIRGVKTNIPFLLTLLTNPVFIDGSYWT | |
| | TFIDDTPELFKMVSSQNRAQKLLHYLADLAVNGSSIKGQMGLPKLH | |
| | TQPSVPTLHDSNGEVIDVLTTPPPSGWRQVLLEQGPAQFAKAVRHY | |
| | KGTLLMDTTWRDAHQSLLATRVRTYDLAAIAPTTAHALSGAFALEC | |
| | WGGATFDVAMRFLHEDPWERLRILRKLVPNIPFQMLLRGANGVAYS | |
| | SLPDNAIDHFVEQAKENGVDIFRVFDALNDLEQLKVGVDAVKKANG | |
| | VVEATICYSGDMLQPGKKYNLDYYLEITDKIVQMGTHILGIKDHAG | |
| | TLKPSAAKLLIGSIRAKYPDLPIHVHTHDSAGTAVASMAACAFAGA | |
| | DVVDVATNSMSGMTSQPSINALLASLDGEIDTGVNVNMVRELDAYW | |
| | AQHRLLYSCFEADLKGPDPEVYEHEIPGGQLTNLLFQAQQLGLGEK | |
| | WAETKRAYRDANYLLGDLVKVTPTSKVVGDLAQFMVSNKLTPDDVR | |
| | RLASSLDFPDSVMDFFEGLIGQPYGGFPEPLRSQVLKNKRRKLTCR | |
| | PGLELAPPDLAGIKEDLQDRFGDIDECDVASYNMYPKVYEDFRKMR | |
| | ETYGDLSVLPTKNFLSPPTVGEEIVVTIEKGKTLIIKPQAIGELNK | |
| | ETGLREVYFDLNGELRKVSVIDKSQKVDTLSKPKADAHDPFQIGAP | |
| | MAGVIIEVKVHKGSLVKKGQPIAVLSAMKMEMVISSPTDCQVKDVL | |
| | VKDGENVDASDLLVFLEESVPPKE* | |

TABLE 8

| | Sequence | SEQ ID NO: |
|---|---|---|
| LelPYC-F | 5′-GACACTTTTTCAAACATGACATCAATTGCTTCATCAAACACTC- | 46 |
| | 3′ | |
| LelPYC-R | 5′-ATCATCCTTGTAATCATGTTTCATAAAATACTGGTGATCAAATC | 47 |
| | TTTG-3′ | |
| LelPYC | MTSIASSNTQDGRINQMRRDSTVLGPMNKILVANRGEIPIRIFRTA | 6 |
| | HELSMQTVAIYSHEDRLSMHRLKADESYAIGKKGQFTPVGAYLQID | |
| | EIIDIAKKHNVNMIHPGYGFLSENSFFAKKVEQNGIVWIGPSHKTI | |
| | DAVGDKVSARTLAIENDVPVVPGTPGPIDDVSDARKFVEKYGTPVI | |
| | IKAAFGGGGRGMRVVREGDDIEDAFKRATSEAKTAFGNGTCFIERF | |
| | LDKPKHIEVQLLADNYGNVIHLFERDCSVQRRHQKVVEIAPAKNLP | |
| | REVRDAILTDAVKLAKSANYRNAGTAEFLVDEQNRHYFIEINPRTQ | |
| | VEHTITEEITGVDIVAAQIQIAAGASLQQLGLLQDKITTRGFAIQC | |
| | RITTEDPAKNFQPDTGKIEVYRSAGGNGVRLDGGNGFAGSIISPHY | |
| | DSMLVKCSCSGSTFEIARRKMIRALIEFRIRGVKTNIPFLLALLTN | |
| | QTFVEGDCWTTFIDDTPSLFQMISSQNRATKLLLYLADLYVNGSSI | |
| | KGQIGYPKLDTEALIPELHEPRTGIAIDVNHTPPPRGWRQVLLEEG | |
| | PAKFAKKVREFKGCLITDTTWRDAHQSLLATRVRTIDLLNIAPTTA | |
| | FALNGAFSLECWGGATFDVCMRFLYEDPWVRLRQLRALVPNIPFQM | |
| | LLRGANGVAYSSLPDNAIDQFVKEAKENGVDIFRVFDALNQLEQLK | |
| | VGVDAVKKAGGVVEATVCYSGDMLKPGKKYNLEYYLNFVDEVVKMG | |
| | THFLGIKDMAGTLKPAAAKLLVGEIRKRHPDLPIHVHTHDSAGTGV | |
| | ASMTECAKAGADVVDAASNSMSGMTSQPSISAILASFEGSIESGLS | |
| | EQLVRELDEYWAQMRLLYSCFEADLKGPDPEVYSHEIPGGQLTNLL | |
| | FQAQQLGLGTKWLETKKAYRIANKILGDLVKVTPTSKVVGDLAQFM | |
| | VSNSLTEEDINRLAGELDFPDSVYDFMEGLMGQPYGGFPEPLRTNM | |
| | LGNKRQKLTQRPGLSLPPVKFDEIKQELQSRYGTQVSETDIASYVM | |
| | YPKVYEAYRKQVEKYGDLSVLPTRYFLKPANIGQEIVVDIEQGKTL | |
| | IIKLLAVGELSEKTGSRETFFELNGEMRSVTVEDKTASVETKTRPK | |
| | AQQPNDVGAPMSGVVIEVRTHKHQEVKKGDPIAVLSAMKMEMIISA | |
| | PVSGVVGDILVKEGDSVDANDLITSILKH* | |

TABLE 9

| | Sequence | SEQ ID NO: |
|---|---|---|
| ScePYC-F | 5′-GACACTTTTTCAAACATGTCGCAAAGAAAATTCGCCG-3′ | 48 |
| ScePYC-R | 5′-ATCATCCTTGTAATCTGCCTTAGTTTCAACAGGAACTTGG-3′ | 49 |
| ScePYC | MSKLAGLRDSSNLLGEKNKLLVANRGEIPIRIFRSAHELSMKTVAI | 7 |
| | YSHEDRLSMHRLKADEAYMIGKEGKYTPVGAYLAIDEILKIAKEHN | |
| | VDFIHPGYGFLSENSEFAQKVEAAGITWIGPSAEVIESVGDKVSAR | |
| | NLAAKADVPVVPGTPGPIDSVEEAQAFVEEYGYPVIIKAAYGGGGR | |
| | GMRVVREGDDIADAFQRATSEAKTAFGNGTCFIERFLNKPKHIEVQ | |
| | LLADGYGNVVHLFERDCSVQRRHQKVVEVAPAKTLPKDVRDAILTD | |
| | AVKLAKVAGYKNAGTAEFLVDDQNRHYFIEINPRIQVEHTITEEIT | |
| | GIDIVAAQIQIAAGASLQELGLLQDKIITRGFAIQCRITTEDPAKN | |
| | FQPDTGRIEVYRSAGGNGVRLDGGNAYAGSIISPHYDSMLVKCSCS | |
| | GSTYEIVRRKMLRALIEFRIRGVKTNIPFLLTLLTNPVFIDGSYWT | |
| | TFIDDTPELFKKVSSQNRAQKLLHYLADLAVNGSSIKGQMGLPKLH | |
| | TQPSVPTLHDSNGEVIDVLTTPPPSGWRQVLLEQGPAQFAKAVRHY | |
| | KGTLLMDTTWRDAHQSLLATRVRTYDLAAIAPTTAHALSGAFALEC | |
| | WGGATFDVAMRFLHEDPWERLRILRKLVPNIPFQMLLRGANGVAYS | |
| | SLPDNAIDHFVEQAKENGVDIFRVFDALNDLEQLKVGVDAVKKANG | |
| | VVEATICYSGDMLQPGKKYNLDYYLEITDKIVQMGTHILGIKDMAC | |
| | TLKPSAAKLLIGSIRAKYPDLPIHVHTHDSAGTAVASMAACAFAGA | |
| | DVVDVATNSMSGMTSQPSINALLASLDGEIDTGVNVNMVRELDAYW | |
| | AQMRLLYSCFEADLKGPDPEVYEHEIPGGQLTNLLFQAQQLGLGEK | |
| | WAETKRAYRDANYLLGDLVKVTPTSKVVGDLAQEMVSNKLTPDDVR | |
| | RLASSLDFPDSVMDFFEGLIGQPYGGFPEPLRSDVLKNKRRKLTCR | |
| | PGLELAPFDLAGIKEDLQDRFGDIDECDVASYNMYPKVYEDFRKMR | |
| | ETYGDLSVLPTKNFLSPPTVGEEIVVTIEKGKTLIIKPQAIGELNK | |
| | ETGLREVYFDLNGELRKVSVIDKSQKVDTLSKPKADAHDPFQIGAP | |
| | MAGVIIEVKVHKGSLVKKGQPIAVLSAMKMEMVISSPTDGQVKDVL | |
| | VKDGENVDASDLLVFLEESVPPKE* | |

TABLE 10

| | Sequence | SEQ ID NO: |
|---|---|---|
| CorPYC-F | 5′-CACTTTTTCAAAcATGATTTTTATAAATTCATCATCAATTTCT | 50 |
| | ACAAAATCTCC-3′ | |
| CorPYC-R | 5′-ATCATCCTTGTAATCGGCTTTCAAAATACTGGTGATTAAATCA | 51 |
| | TTAG-3′ | |
| CorPYC | MIFINSSSISTKSPLNSISLSSTQLLFFCQIYSPPITMTSLQPTAT | 8 |
| | QDGQINQMRRASSVLGPMNKILVANRGEIPIRIFRTAHELSMQTVA | |
| | IYSHEDRLSMHRLKADESYVIGKKGQYSPVGAYLQIDEIIDIAKQH | |
| | NVNMIHPGYGFLSENSEFARKVEENGILWVGPSYKTIDAVGDKVSA | |
| | RTLAIENDVPVVPGTPGPIDDVTDARKFVEKYGFPVIIKAAFGGGG | |
| | RGMRVVREGDDIEDAFKRATSEAKTAFGNGTCFIERFLDKPKHIEV | |
| | QLLADNYGNVIHLFERDCSVQRRHQKVVEIAPAKNLPKEVRDAILT | |
| | DAVKLAKSANYRNAGTAEFLVDEQNRHYFIEINPRIQVEHTITEEI | |
| | TGVDIVAAQIQIAAGASLQQLGLLQDKITTRGFAIQCRITTEDPAK | |
| | NFQPDTGKIEVYKSAGGNGVRLDGGNGFAGSVISPHYDSMLVKCSC | |
| | SGSTFEIARRKMLRALIEFRIRGVKTNIPFLLALLTNETFIKGDCW | |
| | TTFIDDTPSLFQMVSSQNRATKLLSYLADLYVNGSSIKGQIGYPKL | |
| | NEEALIPVLHDPKTGIPIDVAHIPPPRGWRQVLLEEGPDAFAKKVR | |
| | QYNGTLITDTTWRDAHQSLLATRVRTIDLLNIAPTTAFALNGAFSL | |
| | ECWGGATFDVCMRFLYEDPWDRLRKLRALVPNIPFQMLLRGANGVA | |
| | YSSLPDNAIDQFVKEAKGNGVDIFRVFDALNDLEQLKVGIDAVKKA | |
| | GGVVEATVCYSGDMLKPGKKYNLEYYLSVVDEVVKMGTHFLGIKDM | |
| | AGTLKPAAARLLVGEIRNRYPELPIHVHTHDSAGTGVASMTECAKA | |
| | GADVVDAASNSMSGLTSQPSISAILASFEGSIDTGLSESLVRELDT | |
| | YWAQMRLLYSCFEADLKGPDPEVYSHEIPGGQLTNLLFQAQQLGLG | |
| | EKWLQTKETYKIANKILGDLVKVTPTSKVVGDLAQFMVSNSLTEED | |
| | VNRLASELDFFDSVLDFMEGLMGKPYGGFPEPLRTNMLGNKRQKLD | |
| | KRPGLYLKPVDFAAIRKELISRYGSQIDETDVASYVMYPKVFEAFA | |
| | KQVEKYGDLSVLPTRFFLKPANIGEEIVVDIEKGKTLIIKLLAVGE | |
| | ISEKTGTREVFFELNGEMRSVSVEDNTVSVETKTRPKASAPNDVGA | |
| | PMAGVVIEVRTHKHQEVKKGDPIAVISAMKMEMVISAPVSGLVDDL | |
| | LVREGDSVDANDLITSILKA* | |

TABLE 11

| | Sequence | SEQ ID NO: |
|---|---|---|
| CtrPYC-F | 5′-CACTTTTTCAAAcATGTCTGTACCAGTCCAAAAAGCTAAC-3′ | 52 |
| CtrPYC-P | 5′-ATCATCCTTGTAATCATGTTGAATAATGTTAGTAATCAAATCA | 53 |
| | TTAGCATC-3′ | |
| CtrPYC | MSVPVQKANSQDTRISQMRRDSTVLGPMNKILVANRGEIPIRIFRT | 9 |
| | AHELSMQTVAIYSHEDRLSMHRLKADESYVIGKKGQFSPVGAYLQI | |
| | DEIINIAKKHNVNMIHPGYGFLSENSEFARKVEENGIIWIGPSYKT | |
| | IDAVGDKVSARTLAIENDVPVVPGTPGPIETVEEAKAFVDKYGLPV | |
| | IIKAAFGGGGRGMRVVREGDDIEDAFKRATSEAKTAFGNGTCFIER | |
| | FLVKPKHIEVQLLADNYGNVIHLFERDCSVQRRHQKVVEIAPAKSL | |
| | PKHVRDAILTDAVKLAKSANYRNAGTAEFLVDAQDRHYFIEINPRI | |
| | QVEHTITEEITGVDLVAAQIQIAAGASLQQLGLLQDKITTRGFAIQ | |
| | CRITTEDPTKNFQPDTGKIEVYRSAGGNGVRLDGGNGFVGSIISPH | |
| | YDSMLVKCSCSGSTYEIARRKMLRALIEFRIRGVKTNIPFLLALLT | |
| | NDIFIKGDCWTTFIDDTPSLFQMISSQNRATKMLSYLADLVVNGSS | |
| | IKGQVGYPKLETEPVVPDIHEPKTGIVIDVNNTPAPRGWRQVLLEE | |
| | GPEAFAKKVRAFKGTLITDTTWRDAHQSLLATRVRTIDLVNIASTT | |
| | AFALNGAFSLECWGGATFDVCMRFLYEDPWARLRKLRALVPNIPFQ | |
| | MLLRGANGVAYSSLPDNAIDQFVKEAKDNGVDIFRVFDALNDLEQL | |
| | KVGIDAVKKAGGVVEATVCYSGDMLKPGKKYNLEYYLNVVDEIVKL | |
| | GTHFLGIKDMAGTLKPAAAKILIGEIRSRYPDLPIHVHTHDSAGTG | |
| | VASMTQCAVSGADVVDAASNSMSGMTSQPSISALLASFEGSIDTGL | |
| | SESMVRELDNYWAQMRLLYSCFDADLKGPDPEVYQHEIPGGQLTNL | |
| | LFQAQQLGLGSQWVQTKEAYKVANSILGDLVKVTPTSKVVGDLAQF | |
| | MVSNTLTEEDVNRLAAELDFPDSVLDFFQGLMGTPYGGFPEPLRTN | |
| | ILGSKRQKLNERPGLTLPPVDFVGIREELSSRYGGQITETDIASYV | |
| | MYPKVFEQFRNIIDKYGDLSVLPTKYFLKPCAIGEELSVDIEQGKT | |
| | LIIKLLAVGDISDKTGNREVFFELNGEMRSVSVEDKAAAVETRVKP | |
| | KASSPNDVGAPMAGVVIEIRAHRHQEVKKGDPIAVLSAMKMEMVIS | |
| | SPCDGEVGEIVIHEGDSVDANDLITNIIQH* | |

TABLE 12

| | Sequence | SEQ ID NO: |
|---|---|---|
| NcaPYC-F | 5′-CACTTTTTCAAAcATGTCCATAAACAAAAAACTAAACGGCC- | 54 |
| | 3′ | |
| NcaPYC-R | 5′-ATCATCCTTGTAATCAGATGGAGGCGTAGATTCTTCTAAAAG- | 55 |
| | 3′ | |
| NcaPYC | MSINKKLNGLRDNFNLLGTRNKILVANRGEIPIRIFRSAHELSMQT | 10 |
| | VAIYSHEDRLSTHRLKADESYVIGEPHQFTPVGAYLAIDEIINIAK | |
| | RHGVDYIHPGYGFLSENSEFADKVAKAGITWIGPPASVIDSVGDKV | |
| | SARNLATKANVPTVPGTPGPIRTVQDAQDFVNEYGYPVIIKAAFGG | |
| | GGRGMRVVNEGDDLADAFQRASSEALTAFGDGTCFVERFLNKPKHI | |
| | EVQLLADTHGNVIHLFERDCSVQRRHQKVVEVAPAKTLPREVRDAI | |
| | LTDAVKLAKECGYQNAGTAEFLVDDQNRHYFIEINPRIQVEHTITE | |
| | EITGIDIVAAQIQIAAGASLEQLGLLQDKITTRGFAIQCRITTEDP | |
| | AKNFQPDTGALEVYDSAGGNGVRLDGGNAYAGAIISPHYDSMLVKC | |
| | SCSGSTYEVVRRKMIRALIEFRIRGVKTNIPFLLTLLTHPVFIAGD | |
| | YWTTFIDDTPQLFKMVSSQNRAQKLLHYLADLAVNGSSIKGQVGIP | |
| | QLKKVPDIPHIHDAQGNVIDVTNVPPPAGWRQVLLEQGPEEFAKQV | |
| | RQFQGTLLMDTTWRDAHQSLLATRVRTHDLAKIAPTTAHALASAFA | |
| | LECWGGATFDVAMRFLHEDPWKRLRTLRKLVPNIPFQMLLRGANGV | |
| | AYSSLPDNAIDHFVKQAKDNGVDIFRVFDALNDLDQLKVGVDAVKK | |
| | AGGVVEATVCYSGDMLQPGKKYNLDYYLEVTEKIVQMGTHILGIKD | |
| | MAGTMKPAAARLLIGSIRAKYPDLPIHVHTHDSAGTAVASMSAAAY | |
| | AGADVIDVATNSMSGLTSQPSINALLASLDGEINTSINVQHVRELD | |
| | AYWAEMRLLYSCFGADLKGPDPEVYEHEIPGGQLTNLLFQAQQLGL | |
| | GEQWTETKRAYKEANMLLGDIVKVTPTSKVVGDLAQFMVSNKLTSE | |
| | DVKRLANSLDFPDSVMDFFEGLMGQPYGGFPEPLRSDILRNKRRKL | |
| | TCRPGLELVPFELNNIKEDLQTRFGDINECDIASYNMYPKVYEDFQ | |
| | KMKEKYGDLSVLPTKSFLAPAEIGEEILVTIQQGKTLIIKLQAIGD | |
| | LNKETGKREVFFELNGEMRKISVTDKSQKVETVAKPKADTHDPFQI | |
| | GAPMAGVIVEVKVHKGSLVKKGEAVAVLSAMKMEMVISSPADGLVK | |
| | EVLVNESENVDASDLLVLLEESTPPS* | |

TABLE 13

| | Sequence | SEQ ID NO: |
|---|---|---|
| NdaPYC-F | 5′-CACTTTTTCAAAcATGTCGAACAAAAAATTCTGGTATCAGA | 56 |
| | G-3′ | |
| NdaPYC-R | 5′-ATCATCCTTGTAATCTGCTTCTTCCGGAATTTCAGTTTCTT- | 57 |
| | 3′ | |
| NdaPYC | MSNKKFNGIRDNFNLLGPKNKILVANRGEIPIRIFRTSHELSMKTV | 11 |
| | AIYSHEDRLSTHRLKADESYVIGEPNQFTPVGAYLAIDEIINIAKK | |
| | HNVDFIHPGYGFLSENSEFADKVAKNGIAWVGPPASVIDSVGDKVS | |
| | ARHLAERANVPTVPGTPGPIKSAKEAEEFVAKYGFPVIIKAAFGGG | |
| | GRGMRVVREGDDIADAFQRASSEAVTAFGNGTCFVERFLNKPKHIE | |
| | VQLLADNYGNVIHLFERDCSVQRRHQKVVEVAPAKTLPRDIRNAIL | |
| | TDAVKLAKECGYKNAGTAEFLVDDQNRHYFIEINPRIQVEHTITEE | |
| | ITGIDIVAAQIQIAAGASLEQLGLLQDKITTRGFAIQCRITTEDPA | |
| | KNFQPDTGRIEVYGSAGGNGVRLDGGNAYAGAIISPHYDSMLVKCS | |
| | CSGSTYEVVRRKMIRALIEFRIRGVKTNIPFLLTLLTHPVFISGQY | |
| | WTTFIDDTPQLFQMVSSQNRAQKLLHYLADIAVNGSSIKGQVGIPR | |
| | LTKIPDIPPLYDTNNNKIDVLKTPPPAGWRQVLLEKGPVEFAKQVR | |
| | QFQGTLLMDTTWRDAHQSLLATRVRTYDLATIAPTTAHALSGAFAL | |
| | ECWGGATFDVAMRFLHEDPWARLRTLRKLVTNIPFQMLLRGANGVA | |
| | YSSLPDNAIDHFVKQAKDNGVDIFRVFDALNDLDQLKVGVDAVKKA | |
| | GGVVEATLCYSGDMLAKCKKYNLDYYLEVTDKIVQMGTHFLGIXDM | |
| | AGTMKPAAAKLLIGSIRAKYPDLPIHVHTHDSAGTAVSSMAAAAVS | |
| | GADVVDVAINSMSGLTSQPSINALLASLDGEIDTGINVQHVRDLDA | |
| | YWAEMRLLYSCFGADLKGPDPEVYQHEIPGGQLTNLLFQAQQLGLG | |
| | EQWAETKRAYREANHLLGDIVKVTPTSKVVGDLAQFMVSNKLTSDD | |
| | VKRLANSLDPPDSVMDFFEGLMGQPYGGFPEPLRSDILRNKRRKLT | |
| | SRPGLELAPFDLNTIREDLQTRFGDDIDECDVASYNMYPKVYEDFQ | |
| | KIRETYGDLSVLPTKNFLAPAEIGEEIEIVIEQGKTLIVKLQAIGD | |
| | LNKKTGIREVYFELNGEMRKIGTLDRSQKVETVAKPKADGHNPFQI | |
| | GAPMAGVIVEVRVHKGSLVKKGEAVAVLSAMKMEMIISSPTDGQVK | |
| | DVLVNDGENVEASDLLVLLEETEIPEEA* | |

TABLE 14

| | Sequence | SEQ ID NO: |
|---|---|---|
| SkuPYC-F | 5′-CACTTTTTCAAAcATGTCGCAAAAGAAATTCGTGG-3′ | 58 |
| SkuPYC-R | 5′-ATCATCCTTGTAATCTGCTTTAGTTTCAGCTGGAACTTGAT- | 59 |
| | 3′ | |
| SkuPYC | MSQKKFAGLRDNFNLLGEKNKILVANRGEIPIRIFRTAHELSMQTV | 12 |
| | AIYSHEDRLSTHKQKADEAYVIGEAGQYTPVGAYLAIDEIISIAQK | |
| | HQVDFIHPGYGFLSENSEFADKVAKAGITWIGPPAEVINSVGDKVS | |
| | ARNLAARANVPTVPGTPGPIESVQEALDFVGEYGYPVIIKAAFGGG | |
| | GRGMRVVREGDDVADAFQRATSEARTAFGNGTCFVERFLVKPKHIE | |
| | VQLLADNHGNVVHLFERDCSVQRRHQKVVEVAPAKTLPREVRDAIL | |
| | TDAVKLAKECGYRNAGTAEFLVDNQNRHYFIEINPRIQVEHTITEE | |
| | ITGIDIVAAQIQIAAGASLPQLGLFQDKITTRGFAIQCRITTEDPA | |
| | KNFQPDTGRIEVYRSAGGNGVRLDGGNAYAGTIISPHYDSMLVKCS | |
| | CSGSTYEIVRRKMIRALIEFRIRGVKTNMPFLLTLLTHPVFIDGTY | |
| | WTTFIDDTPQLFQMVSSQNRAQKLLHYLADVAVNGSSIKGQIGSPK | |
| | LGSNPSIPHLHDSQGNVINVTKTTPPSGWRQVLLEKGPAEFAKQVR | |
| | QFNGTLLMDTTWRDAHQSLLATRVRTHDLATIAPTTAHALAGAFAL | |
| | ECWGGATFDVAMRFLHEDPWERLRKLRALVPNIPFQMLLRGANGVA | |
| | YSSLPDNAIDHFVKQAKDNGVDIFRVFDALNDLDQLKVGVDAVKKA | |
| | GGVVEATVCFSGDMLQPGKKYNLDYYLEIAEKIVKMGTHILGIKDM | |
| | AGTMKPAAAKLLIGSLRAKYPDLPIHVHTMDSAGTAVASMAACALA | |
| | GADVVDVAINSMSGLTSQPSINALLASLEGNIDTGINVEHVRELDA | |
| | YWAEMRLLYSCFEADLKGPDPEVYQHEIPGGQLTNLLFQAQQLGLG | |
| | EQWAETKRAYREANYLLGDIVKVTPTSKVVGDLAQFMVSNKLTPDD | |
| | VKRLANSLDFPDSVMDFFEGLIGQPYGGFPEPFRSDVLRNKRRKLT | |
| | CRPGLELEPFDLEKIREDLQNRFGDINECDVASYKMYPRVYEDFQK | |
| | MRETYGDLSVLPTRSFLSPLETDEEIEVIIEQGKTLIIKLQAVGDL | |
| | NKKTGEREVYFDLNGEMRKIRVVDRSLKVATVTKSKADMHDPLHIG | |
| | APMAGVIVEVKVHKGSLIKKGQPVAVLSAMKMEMIISSPSDGQVKE | |
| | VFVSEGENVDSSDLLVLLEDQVPAETKA* | |

TABLE 15

| | Sequence | SEQ ID NO: |
|---|---|---|
| SpoPYC-F | 5′-CACTTTTTCAAAcATGACTTCTAAATATGATGCGTTGTTGC- | 60 |
| | 3′ | |
| SpoPYC-R | 5′-ATCATCCTTGTAATCCTCGTGTTCAAGAACAGCACACA-3′ | 61 |
| SpoPYC | MTSKYDALLHNQSTNTNPFSKLQDRSSLLGEKFTKVLVANRSEIAI | 13 |
| | RVFRTAHELSMHTVAIYSYEDRLSMHRQRADESYPIGKVGQYSPVG | |
| | AYLAIDEIVSIAKRTGANLVHPGYGFLSENAEFARKVNEAGMQFVG | |
| | PSPEVIDSLGDKTKARAIAIRCGVPVVPGTPGPVEHYEEAEAFVKE | |
| | YGLPVIIKAAMGGGGRGMRVVRSADTLKESFERARSEALASFGDGT | |
| | VFIERFLDKPKHIEIQLMADKAGNVIHLHERDCSVQRRHQKVVEIA | |
| | PAKDLDPKIRQALYDDAIKIAKEVKYCNAGTAEFLLDQKGRHYFIE | |
| | INPRIQVEHTITEEITGVDIVSAQLHVAAGFTLPEIGLTQDKISTR | |
| | GFAIQCRVTTEDPNNGFAPDIGKIEVYRSAGGNGVRLDGANGFAGS | |
| | VITPHYDSMLVKCTCHDATYEYTRRKMIRSLIEFRVRGVKTNIPFV | |
| | LRLLMHDIFIQGNCWTTFIDDTPELFQLYRSRNRAQKLLAYLGDLA | |
| | VNGSSIKGQNGEPALKSEIVMPVLLDSTGNQIDVSHPSEKGWRKLL | |
| | LDNGPAAFAKAVRNHKRGLIMDTTWRDAHQSLLATRVRTIDLVNIA | |
| | PYTSHALASAYSLEMWGGATFDVSMRFLHECPWDRLRRLRKLVPNI | |
| | PFQMLLRGANGLCYSSLPDNVIYFFCEQAKKNGIDIFRVFDALNDV | |
| | NNLSLGIDAAKRAGGVVEATMCYSGDMLNPKKKYNLDYYVNLVDKM | |
| | VEMGIHILGIKDMAGVMKPKAARLLISAIREKHPELPIHVHTHDSA | |
| | GTAVASMAAALEAGADVVDVATDSMSGLTSQPSFGAVLASVDGTDK | |
| | QLEFDNNQLREIDSYWAQMRLLYSPFESEIKGTDSDVYNHEIPGGQ | |
| | LTNLKFQATSLGLGTQWAETKKAYIEANKLLGDIIKVTPDSKVVGD | |
| | LAQFMVQNKLSAEDVENRATTLDFPASVLDFFQGLMGQPYGGFPEP | |
| | LRTNVLKGRRQPLTDRPGKFLPAADFDAIRKLLSEKFGVSSDCDIA | |
| | AYTQFPGVFEEYRQFVDRYGDLTTVPTKFFLSRPEMNEEMHVEIDQ | |
| | GKTLIVKFVALGPLNPRTGQREVYFELNGENRHVTVEDKKAAIETV | |
| | TRPRADPGNPGHVAAPMSGTIVEIRVKEGAKVKKGDIIAVLSAMKM | |
| | EIVISAPHSGVLKSLAVVQGDSVNGGDLCAVLEHE* | |

TABLE 16

| | Sequence | SEQ ID NO: |
|---|---|---|
| TblPYC-F | 5′-CACTTTTTCAAAcATGACAAATAAGAAATTCTCTGGTCTAAGA | 62 |
| | GA-3′ | |
| TblPYC-R | 5′-ATCATCTTGTAATCTTTTGGTGGTTCTGGATCTTCTAATGTG- | 63 |
| | 3′ | |
| TblPYC | MTNKKFSGLRDNFSLLGEKNKVLVANRGEIPIRIFRTAHELSMRTV | 14 |
| | AIYSHEDRLAMHRLKADESYVIGAENQYTPVGAYLAIDEIINIAKK | |
| | HNVDFIHPGYGFLSENSEFARKVTESGITWIGPPAAVIDSVGDKVS | |
| | ARNLAAKVNVPTVPGTPGPIDTVEEAKAFVEKHGFPVIIKAAFGGG | |
| | GRGMRVVREGDDIADAFQRATSEARTAFGNGTCFIERFLDHPKHIE | |
| | VQLLADNYGNVVHLFERDCSVQRRHQKVVEVAPAKTLPREVRDSIL | |
| | TDAVKLAKEAGYTNAGTAEFLVDNLGRHYFIEINPRIQVEHTITEE | |
| | ITGIDIVAAQIQIAAGASLEDLGLLQDRITYRGFAIQCRITTEDPS | |
| | KNFQPDTGRLEVYRSAGGNGVRLDGGNAYVGATISPHYDSMLVKCS | |
| | CSGSTYEIVRRKMLRSLIEFRIRGVKTNIPFLLTLLTHPVFVSGDY | |
| | WTTFIDDTPQLFQMVSSKNRAQKLLHYLADLAVNGSSIKGQIGLPK | |
| | LATRPDIPALHDKNGEVINVTNAEPPSGWRKALLEKGPKEFAKQVR | |
| | EFNGCLIMUTTWRDAHQSLLATRLRTYDLAAIAPTTSFALAGAFAL | |
| | ECWGGATFDVAMHFLHEDPWERLRTLRKLVPNIPFQMLLRGANGVA | |
| | YSSLPDNAINHFVKQAKDNGVDIFRVFDSLNDVEQLKVGIEAVKLA | |
| | GGVVEATMCYSGDMLQPGKKYNLDYYLELSEKIVGLGTHILGIKDM | |
| | AGTMKPAAAKLLISSLRKKYPDLPIHVHSHDSAGTAVTSMVACAKY | |
| | GADVVDVAINSMSGLTSQGSINALLASLDGDIETNINAQHVRELDA | |
| | YWAEMRLLYSCFEADLKGPDPEVYEHEIPGGQLTNLLFQAQQLGLG | |
| | EKWKETKRAYREANYLLGDIVKVTPTSKVVGDLAQFMVTNKLTSDD | |
| | VRRLANSLDFPDSVMDFFEGLLGQPYGGFPEPLRTDILKGKRKKMT | |
| | SRPGLELQPFDIAAIKEDLQNRFGDIDECDVASYNMYPKVYEDFQK | |
| | IREKYGDLSVLPTKNFLAPPVTGEEIEVTIEQGKTLIIKCQAVGDL | |
| | NKATGTREVYFELNGELRKIPVVDRSLKVDIVAKPKADTHDPYQIG | |
| | APMAGVIVEVKVHKGSLVKKGQPVAVLSAMKMEMVISSPSDGLVKN | |
| | VTVADGENVDASDLLITLEDPEPPK* | |

TABLE 17

| | Sequence | SEQ ID NO: |
|---|---|---|
| TdePYC-F | 5′-CACTTTTTAAAcATGTCCAAGAACAAGAGGTTTGCT-3′ | 64 |
| TdePYC-R | 5′-ATCATCCTTGTAATCGTTTTCAGCTGGGACAGCCTC-3′ | 65 |
| TdePYC | MSKNKRFAGLRDNFSLLGEKNKLINANRGEIPIRIFRSAHELSMRT | 15 |
| | VATYSHEDRLSMHKLKSDEAYVIGEEGKYTPVGAYLAIDEIIEIAK | |
| | QHNVDFIHPGYGFLSENSEFAKKVADNGITWIGPPADVIDAVGDKV | |
| | SARNLALKADVPTVPGTPGPIENVQEAKAFVEKYGYPVIIKAAFGG | |
| | GGRGMRVVREGDDIEDAFQRATSEARTAFGNCTCFIERFLVKPKHI | |
| | EVQLLADNYGNVVHLFERDCSVQRRHQKVVEVAPAKTLPVEVRNAI | |
| | LTDAVKLAHTAGYRNAGTAEFLVDNQNRHYFIEINPRIQVEHTITE | |
| | EITGIDIVAAQIQIAAGASLEQLGLMQDRITTRGFAIQCRITTEDP | |
| | SKNFQPDTGRLEVYRSAGGNGVRLDGGNAFAGAIISPHYDSMLVKC | |
| | SCSGSTYEIVRRKMIRALIEFRIRGVKTNIPFLLTLLTNPVFVNGD | |
| | YWTTEIDDTPQLFQMISSQNRAQKLLHYLADLAVNGSSIKGQLGVP | |
| | KLTTHPTIPHLHDAQGEIINVNSAAPPAGWRQILLDYGPKEFAKQV | |
| | RDFDGTLIMDTTWRDAHQSLLATRVRTYDLAAIAPTTAHALSGAFA | |
| | LECWGGATFDVAMRFLHEDPWERLRILRKLVPNIPFQMLLRGANGV | |
| | AYSSLPDNAIDHFVKQAKENGVDIFRVFDALNDLEQLKVGVDAVKK | |
| | AGGVVEATVCYSGDMLQPGKKYNLDYYLEVTGKIVEHGTHILGIKD | |
| | MAGTMKPSAARLLIGSIRAKYPDLPIHVHTHDSAGTAVASNAACAL | |
| | AGADVVDVATNSMSGLTSQPSMNALLASLDGQINTNIDVQHTRELD | |
| | AYWAEMRLLYSCFEADLKGPDPEVYEHEIPGGQLTNLLFQAQQLGL | |
| | GEKWAETKRAYKEANYLLGDLVKVTPTSKVVGDLAQFMVSNKLTSG | |
| | DVKRLASSLDFPDSVMDFFEGLIGQPYGGFFEPLRTDILKNKRRKL | |
| | TCRPGLELAPFDLEKIREDLQDRFGDIDECDVASYNMYPKVYEDFH | |
| | KIKERYGDLSVLPTKNFLSPPKIGEEIEVTIEKGKTLIIKHQAVGD | |
| | LNKETGVREVYFELNGELRKIPVVDKSQKVESVAKPKADGHDPLQV | |
| | GAPMAGVIVEVKVHKGSLVKKGEPVAVLSAMKMEMVISAQADGQVK | |
| | EVYINDGDNVDASDLLVVLEEAVPAEN* | |

TABLE 18

| | Sequence | SEQ ID NO: |
|---|---|---|
| ZroPYC-F | 5′-CACTTTTTCAAAcATGAGTACCAAGAAGITCTCTGGTCT-3′ | 66 |
| ZroPYC-R | 5′-ATCATCCTTGTAATCATTCTCCTTTGGTGGGACGG-3′ | 67 |
| ZroPYC | MSTKKFSGLRDNFSLLGEKNKILVANRGEIPIRIFRSAHELSMRSV | 16 |
| | AVYSHEDRLSMHRLKADEAYVIGEEGKYTPVGAYLAIDEIIAIAKK | |
| | HNVDFIHPGYGFLSENSEFAKKVEANGITWIGPPAEVIDSVGDKVS | |
| | ARNLAAKAHVATVPGTPGPIESVDEAIEFTKKYGFPVIIKAAFGGG | |
| | GRGMRVVREGDDVADAFQRASSEARSSFGNGTCFMERFLVKPKHIE | |
| | VQLLADNYGNVVHLFERDCSVQRRHQKVVELAPAKALPVETRNAIL | |
| | TDAVKLAKTAGYRNAGTAEFLVDNENRHYFIEINPRIQVEHTITEE | |
| | ITGIDIVAAQICIAAGASLDQLGLLQDRITTRGFAIQCRITTEDPS | |
| | KNFQPDTGRIEVYRSSGGNGVRLDGGNTYAGAVISPHYDSMLVKCS | |
| | CSGSTYEIVRRKMIRALVEFRIRGVKTNIPFLLTLLTHPTFVQGQF | |
| | WTTFIDDTPQLFQMISSQNRAQKLLHYLADLAVNGSSIKGQVGLPK | |
| | LKTHPTVPRLHGSNGEVIDVLATPPPSGWRQVLLEHGPEEFAKRVR | |
| | QFNGCLIMDTTWRDAHQSLLATRVRTYDLAAIAPTTAHALSGAFSL | |
| | ECWGGATFDVAMRFLHEDPWERLRILRRLVPNVPFQMLLRGANGVA | |
| | YSSLPDNAIDHFVKQAKENGVDIFRVFDALNDLDQLKVGVDAVKKA | |
| | GGVVEATVCYSGDMLQPGKKYNLDYYLEVTEQIVKMGTHFLGIKDM | |
| | AGTMKPAAAKLLVGSIRAKYPNLPIHIHTHDSAGTGVASMAACAVS | |
| | GADVVDVAVNSMSGLTSQPCMNALLASLDGEINTGINANYARELDA | |
| | YWAEMRLLYSCFDTDLKGPDPEVYEHEIPGGQLTNLLFQAQQLGLG | |
| | EKWIETKKAYKEANYLLGDLVKVTPTSKVVGDLAQFMVSNKLTSDD | |
| | VRRLANSLDFPDSVMDFFEGLIGQPYGGFPEPLRSDVLRNKRRKLT | |
| | NRPGLDLAPFDLEAVREDLQDRFGDTDECDVASYNMYPKVYEDFQK | |
| | IKEQFGDLSVLPTKNFLSPPGIGEEIEVTIEQGKTLIIKHQAVGDL | |
| | NKETGMREVYFELNGELRKIPVVDRSQKVEIVSKPKADGHDPYQVG | |
| | APMAGVIVEIKVHKGSLIKKGQPVAVLSAMKMEMVISAQTDGQVKE | |
| | VLANGGDNVDASDLLVTLEDAVPPKEN* | |

TABLE 19

| | Sequence | SEQ ID NO: |
|---|---|---|
| AfuMDH-F | 5′-ACTTTTTCAAACATGAAACTCGGTTTTGTTGGTGC-3′ | 68 |
| AfuMDH-R | 5′-ATCATCATCATCCTTGTAATCATATCCGAGTTCCTCAAGCCTC | 69 |
| | TC-3′ | |
| AfuMDH | MKLGFVgAGRVGSTSAFTCLLNLDVDEIALVDIASDLAVGEAMDLA | 17 |
| | HAAAGIDKYPKIVGGADYSLLKGSEIIVVTAGLARKPGMTRLDLAH | |
| | KNAGIIKDIAKKIVENAPESKILVVTNPMDVMTYIMWKESGKPRNE | |
| | VFGMGNQLDSQRLKERLYNAGARNIRRAWIIGEHGDSMFVAKSLAD | |
| | FDGEVDWEAVENDVRFVAAEVIKRKGATIFGPAVAIYRMVKAVVED | |
| | TGEIIPTSMILQGEYGIENVAVGVPAKLGKNGAEVADIKLSDEEIE | |
| | KLRNSAKILRERLEELGY* | |

TABLE 20

| | Sequence | SEQ ID NO: |
|---|---|---|
| CliMDH-F | 5′-ACTTTTTCAAACATGAAAGCACCTTCGCGT-3′ | 70 |
| CliMDH-R | 5′-ATCATCATCATCCTTGTAATCGGGCAGAAGGTCTGCCACG-3′ | 71 |
| CliMDH | MKAPVRVTVTGAAGQIGYALLFRIASGAMLGDDQPVILHLLDITPA | 18 |
| | MDALEGVRMELDDCAFPLLAGIVCTDDPNVGFKDADYALLVGARPR | |
| | GPGMERKDLLEANAAIFSVQGKAINDHASRDIRVLVVGNPANTNAL | |
| | ITQRNAPDIDPRNFTAMMRLDHNRAKTQIAQKLDAPVTAVSTMTVW | |
| | GNHSATQYPDLHHSTVNGKVAVDAVEQQWYEDEFIPTVQORGAAII | |
| | KARGASSAASAANAAIDHMRDWALGTPEGDWVSMGVYSDGSYGIAE | |
| | GLIYSFPCRCSGGDWSIVQGLEINDFSRGRMQATEQELTEERDAVA | |
| | DLLP* | |

TABLE 21

| | Sequence | SEQ ID NO: |
|---|---|---|
| DacMDH-F | 5′-ACTTTTTCAAACATGAGCAAGAAGCCCGTTCG-3′ | 72 |
| DacMDH-R | 5′-ATCATCATCATCCTTGTAATCCAGCAGGTGCTTGACGCC-3′ | 73 |
| DacMDH | MSKKPVRVAVTGAAGQIGYALLFRIASGEMLGKDQPVILQLLEIPC | 19 |
| | EKAONALKGVIMELEDCAFPLLAGIEAHSDPLQAFKDTDYALLVGA | |
| | RPRGPGMERADLLAANAQIFTAQGKALNAAASRNVKVLVVGNPANT | |
| | NAYIAMKSAPDLPAKNFTAMLRLDHNRAASQLAAKAGFKVGDIRKL | |
| | TVWGNHSPTMYADYRFATVNGESVKAKINDQAWNKDVFLPTVGKRG | |
| | AAIIAARGLSSAASAANAAIDHMRDWALGSGGEWVTMGVPSNGEYG | |
| | IPAGIVFGFPVTTENGEYKIVEGLEIDAFSQECIDKTLAELQGEQD | |
| | GVKHLL* | |

TABLE 22

| | Sequence | SEQ ID NO: |
|---|---|---|
| HelMDH-F | 5′-ACTTTTTCAAACATGAAAGACCCCGTCCGTATAGC-3′ | 74 |
| HelMDH-R | 5′-ATCATCATCATCCTTGTAATCGCCAAGCAGATGCTCGACG-3′ | 75 |
| HelMDH | MKDPVRIAITGAAGQISYSLAFRIASGDMLGKDQPVILQLLEIPPA | 20 |
| | MDALNGVVMELNDCAFPLVQDIVASDDPNVAFKDADFALLVGARPR | |
| | GPGMERKDLLEANAAIFSVQGKALNDHASRDVRVLVVGNPANTNAL | |
| | IASCNAPDLDAGQFTAMMRLDHNRAKTQLAQKVGKHNTDVESMIVW | |
| | GNHSATQYPDLAHCKVDGKPALELVDQAWYENDFIPTVQQRGAAII | |
| | KARGASSAASAASAAIDHMRDWALGSDGIVSMGIPADGSYGIEPGI | |
| | MYSYPVVCKNGQYEIVQDLEIGDFSRERMNATEQELREERDAVEHL | |
| | LG* | |

TABLE 23

| | Sequence | SEQ ID NO: |
|---|---|---|
| SpuMDH-F | 5′-ACTTTTTCAAACATGAAAGTTGCTGTACTTGGTGC-3′ | 76 |
| SpuMDH-R | 5′-ATCATCATCATCCTTGTAATCTTTAACGAAATCAACGCCTAGT | 77 |
| | TTGATATC-3′ | |
| SpuMDH | MKVAVLGAAGGIGQALALLLKTQLPAGSKLSLYDIAPVTPGVAVDL | 21 |
| | SHIPTAVEIKGFAGEDPTPALVGADVVLISAGVARKPGMDRSDLFN | |
| | INAGIVRNLIEKVAATCPKALVGIITNPVNTTVAIAAEVMKKAGVY | |
| | DKNRLFGVTTLDVIRSETFIAELKGLNVADVKINVIGGHSGVTILP | |
| | LLSQVEGVTFSDEEVASLTKRIQNAGTEVVEAKAGGGSATLSMGQA | |
| | ACRFGMSLVRGLQGEANVVECAYVDGGSEHAEFFAQPVLLGKNGIE | |
| | KVLPYGEVSAFEANARDSMLDTLKGDIKLGVDFVK* | |

The name and host of each of the manufactured transformants and the name of the introduced foreign gene are shown in Table 24.

TABLE 24

| | | Name of introduced |
|---|---|---|
| Name of transformant strain | Name of host strain | foreign genes |
| 1 | IGF836 | AniPYC |
| 2 | IGF836 | BbrPYC |
| 3 | IGF836 | DhaPYC |
| 4 | IGF836 | KlaPYC |
| 5 | IGF836 | LthPYC |
| 6 | IGF836 | LelPYC |
| 7 | IGF836 | ScePYC |
| 8 | IGF836 | CorPYC |
| 9 | IGF836 | CtrPYC |
| 10 | IGF836 | NcaPYC |
| 11 | IGF836 | NdaPYC |
| 12 | IGF836 | SkuPYC |
| 13 | IGF836 | SpoPYC |
| 14 | IGF836 | TblPYC |
| 15 | IGF836 | TdePYC |
| 16 | IGF836 | ZroPYC |
| 17 | IGF836 | AfuMDH |
| 18 | IGF836 | CliMDH |
| 19 | IGF836 | DacMDH |
| 20 | IGF836 | HelMDH |
| 21 | IGF836 | SpuMDH |
| ASP4491 | IGF836 | ScePYC, DacMDH |

<Confirmation of PYC Expression>

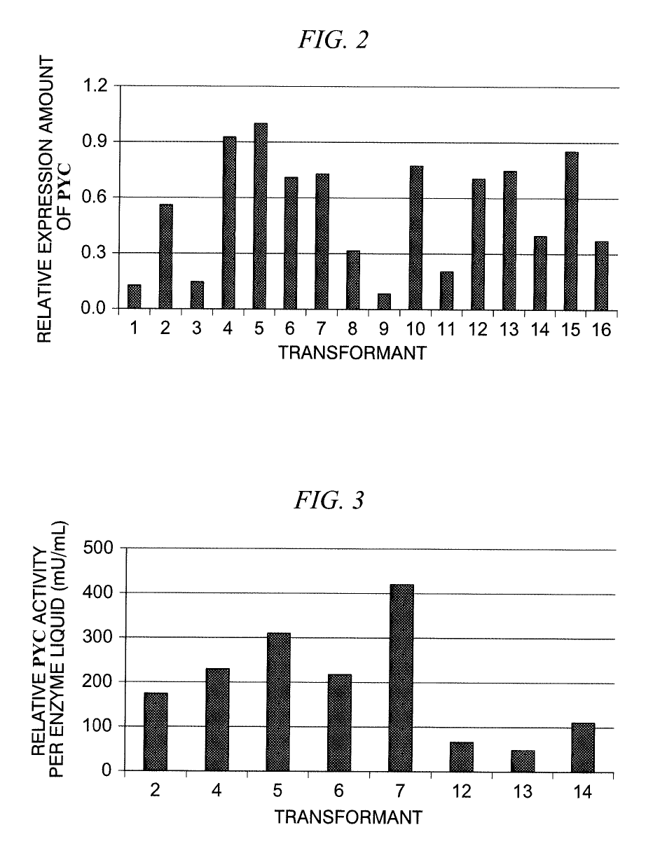

For 16 kinds of transformant into which only the PYC gene was introduced, an expression amount of PYC was confirmed. Specifically, each of the transformants was shake-cultured for 44 hours in 20 mL of a YPD6 medium, and the grown microbial cells were then collected and pulverized using a multibead shocker. The obtained microbial cell lysate was purified according to a protocol by using ANTI-FLAG (registered trademark) M1 Agarose Affinity Gel (manufactured by Sigma-Aldrich Co. LLC.). SDS-PAGE was performed using a purified enzyme liquid, and the detected band was digitized using an image analysis device LAS 4000, thereby calculating a relative expression amount. The results of the PYC expression amount (relative values) for each of the transformants are shown in FIG. 2. The results show that the expression of PYC was confirmed in all of the transformants.

<Confirmation of PYC Activity>

Among the 16 kinds of transformant into which only the PYC gene was introduced, 8 kinds of transformant caused to express BbrPYC, KluPYC, LthPYC, LelPYC, ScePYC, SkuPYC, SpoPYC, or TblPYC were investigated regarding PYC activity. Specifically, the aforementioned purified enzyme liquid was mixed with a reaction solution kept at 30° C. (100 mM Tris-HCl (pH 7.5), 2 mM ATP, 100 mM MgCl₂, 100 mM KCl, 5 mM Pyruvic acid, 5 mM NaHCO₃, 0.1 mM Acetyl-CoA, 0.15 mM NADH), and then measured over time by using an absorptiometer having a mixing function. From the obtained temporal change of absorbance, a relative activity (mU/mL) per enzyme liquid was calculated. The results of the PYC activity per enzyme liquid calculated for each of the transformants are shown in FIG. 3. The results show that the PYC activity was confirmed in all of the transformants.

<Confirmation of MDH Expression>

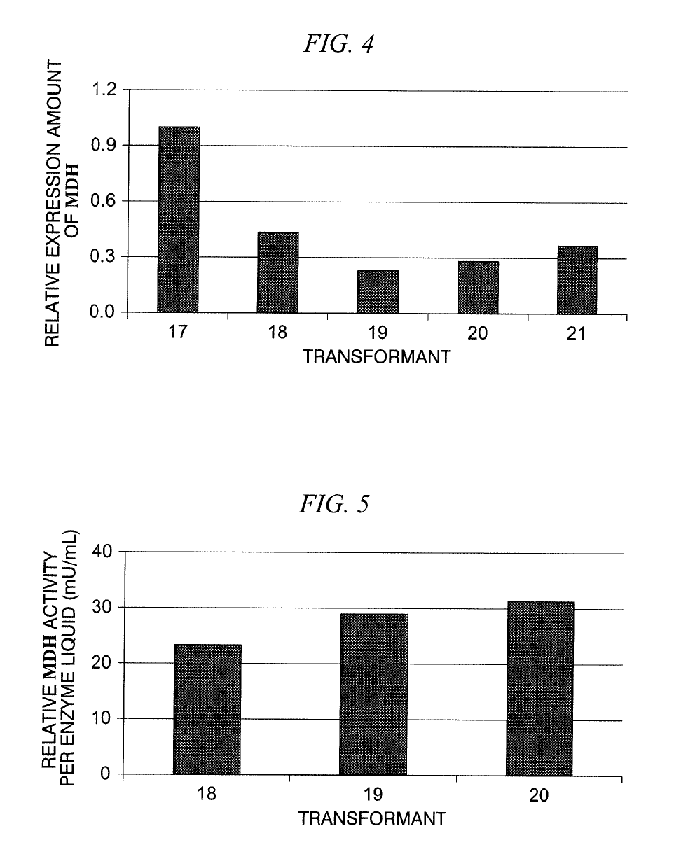

For 5 kinds of transformant into which only the MDH gene was introduced, an expression amount of MDH was confirmed. Specifically, each of the transformants was cultured in the same manner as described in <Confirmation of PYC expression>, a purified enzyme liquid was prepared, SDS-PAGE was performed using the enzyme liquid, and a relative expression amount was calculated. The results of the MDH expression amount (relative values) for each of the transformants are shown in FIG. 4. The results show that the expression of MDH was confirmed in all of the transformants.

<Confirmation of MDH Activity>

Among 5 kinds of transformant into which only the MDH gene was introduced, 3 kinds of transformant caused to express CliMDH, DacMDH, or HelMDH were investigated regarding MDH activity. Specifically, the aforementioned purified enzyme liquid was mixed with a reaction solution kept at 30° C. (90 mM Tris-HCl (pH 9.0), 0.5 mM Oxaloacetate, 0.25 mM NADH) and then measured over time by using an absorptiometer having a mixing function. From the obtained temporal change of absorbance, a relative activity (mU/mL) per enzyme liquid was calculated. The results of the MDH activity per enzyme liquid calculated for each of the transformants are shown in FIG. 5. The results show that the MDH activity was confirmed in all of the transformants.

### Example 3 Preparation of Mae2 Deletion Strain

By deleting an mae2 gene from the ASP4491 strain into which an ScePYC gene and a DacMDH gene were introduced, a deletion strain (ASP4964 strain) was prepared. A method for preparing an mae2 deletion fragment was the same as the method for preparing the deletion fragment from which pdc2 was deleted.

The ASP4491 strain (genotype: h⁻, leu1-32, ura4-D18, Ade6-M216, Δpdc2, +ScePYC, +DacMDH) of S. pombe was transformed according to the Latour method, thereby preparing an ASP4964 strain from which an mae2 gene (strain name: SPCC794. 12c) was deleted.

For preparing a deletion fragment, total genomic DNA prepared from an ARC032 strain of S. pombe by using DNeasy (manufactured by QUIAGEN) was used as a template, and 8 kinds of synthetic oligo DNA (manufactured by Operon Biotechnologies) having the base sequences shown in Table 25 were used.

TABLE 25
Oligo DNA for preparing mne2 deletion fragment

| Oligo DNA | Base sequence | SEQ ID NO: |
|---|---|---|
| UF | 5′-AGGCTTTGATAGCCACTGGT-3′ | 78 |
| UR | 5′-TGGGATTTGTAGCTAAGCTTTAAAA | 79 |
| | TAAAAGGCTTTATAC-3′ | |
| OF | 5′-TTCGTCAATATCACAAGCTTAGGTC | 80 |
| | GACTGGGCTAATCG-3′ | |
| OR | 5′-TAAAATAAAAGGCTTTATACACGCAT | 81 |
| | TTTCAAACTTCAAG-3′ | |
| DF | 5′-CTTGAAGTTTGAAAATGCCTGTATAA | 82 |
| | AGCCTTTTATTTTA-3′ | |
| DR | 5′-TTCATTCAATACATAACGGTTTACG | 83 |
| | GT-3′ | |
| FF | 5′-ATTCGTGAAATGAGCAAGCA-3′ | 84 |
| FR | 5′-TGCGATTTACTATTTGTTTGTTT | 85 |
| | CA-3′ | |

### Example 4 Preparation of Fum1 Deletion Strain

By deleting a fum1 gene from the ASP4491 strain into which an ScePYC gene and a DacMDH gene were introduced, a deletion strain (ASP4933 strain) was prepared. fum1 is an enzyme which produces fumaric acid by using malic acid as a matrix. A method for preparing an fum1 deletion fragment is the same as the method for preparing the deletion fragment from which pdc2 was deleted.

The ASP4491 strain of S. pombe was transformed according to the Latour method, thereby preparing an ASP4933 strain from which an fum1 gene (strain name: SPCC18. 18c/SPCC290. 01c) was deleted.

For preparing a deletion fragment, total genomic DNA prepared from an ARC032 strain of S. pombe by using DNeasy (manufactured by QUIAGEN) was used as a template, and 8 kinds of synthetic oligo DNA (manufactured by Operon Biotechnologies) having the base sequences shown in Table 26 were used.

TABLE 26
Oligo DNA for preparing fum1 deletion fragment

| Oligo DNA | Base sequence | SEQ ID NO: |
|---|---|---|
| UF | 5′-CTCAAGTAATGGGCAATCATGC | 86 |
| | CA-3′ | |
| UR | 5′-TGGGATTTGTAGCTAAGCTTAGAGT | 87 |
| | GGTAAAAAATTATAC-3′ | |
| OF | 5′-TTCGTCAATATCACAAGCTTAATAAA | 88 |
| | ATACACAAAACTGT-3′ | |
| OR | 5′-AGAGTGGTAAAAAATTATACAGCCA | 89 |
| | TGTGGGTTATTTTAA-3′ | |
| DF | 5′-TTAAAATAACCCACATGGCTGTATAA | 90 |
| | TTTTTTACCACTCT-3′ | |
| DR | 5′-GAGCAACCGAATATCAAGGAAATACA | 91 |
| | CA-3′ | |
| FF | 5′-AGGCGAATTGATCCTTCCTGCTA-3′ | 92 |
| FR | 5′-TGGTAAGCCTGGTATGAGTTCTATAC | 93 |
| | TAT-3′ | |

<Confirmation of Malic Acid Producibility>

For a wild strain (ARC010 strain) of S. pombe, the PDC2 gene deletion strain (ASP4590 strain, ΔPDC2), the ASP4491 strain obtained by introducing an ScePYC gene and a DacMDH gene into the ASP4590 strain, the ASP4964 strain (ΔPDC2, Δmae2, +ScePYC, +DacMDH) obtained by deleting an mae2 gene from the ASP4491 strain, and the ASP4933 strain (ΔPDC2, Δfum1, +ScePYC, +DacMDH) obtained by deleting an fum1 gene from the ASP4491 strain, a malic acid production rate was investigated.

Specifically, the microbial cells were seeded into a YES plate and cultured for 96 hours at 30° C., thereby obtaining a colony. The obtained colony was subcultured in 5 mL of a YES medium (test tube) and shake-cultured for 24 hours at 30° C. The obtained microbial solution was subcultured in 100 mL of a YPD6 medium (Sakaguchi flask) and shake-cultured for 44 hours at 30° C.

The microbial cells obtained in this way were collected, added to 3 mL of a fermentation medium (100 g/L glucose, 111 g/L calcium carbonate) in the test tube so as to yield 36 g (weight of dry microbial cell)/L, and fermented at 30° C. under shaking conditions. A sample was collected in time from the fermented liquid.

For the obtained sample, a glucose concentration and an ethanol concentration were measured using a biosensor BF-7 (manufactured by Oji Scientific Instruments) based on an enzyme electrode method, and a malic acid concentration was measured by HPLC. During the HPLC measurement, a high-performance liquid chromatograph Prominence (manufactured by Shimadzu Corporation) was used, Aminex HPX-87H 300×7.8 mm (manufactured by Bio-Rad Laboratories, Inc.) was used as a column, an injection volume was set to be 10 μL, 10 mM H₂SO₄ was used as a solvent, a flow rate was set to be 0.6 mL/min, a measurement time was set to be 35 minutes, a measurement temperature was set to be 60° C., and diode array detector (210 nm) and a differential refractometric detector were used for detection. Each concentration is a concentration per culture solution or fermented liquid.

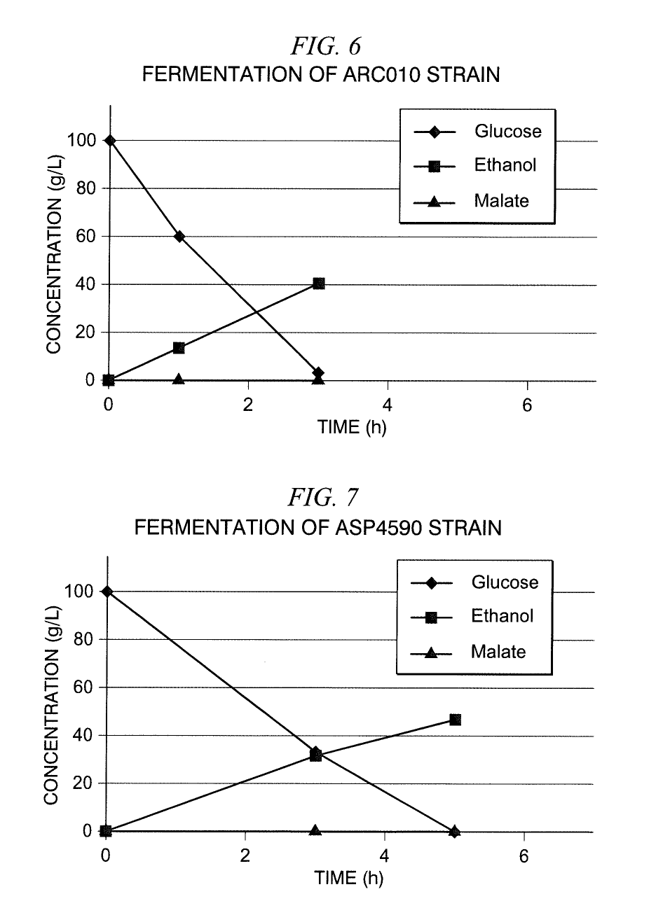

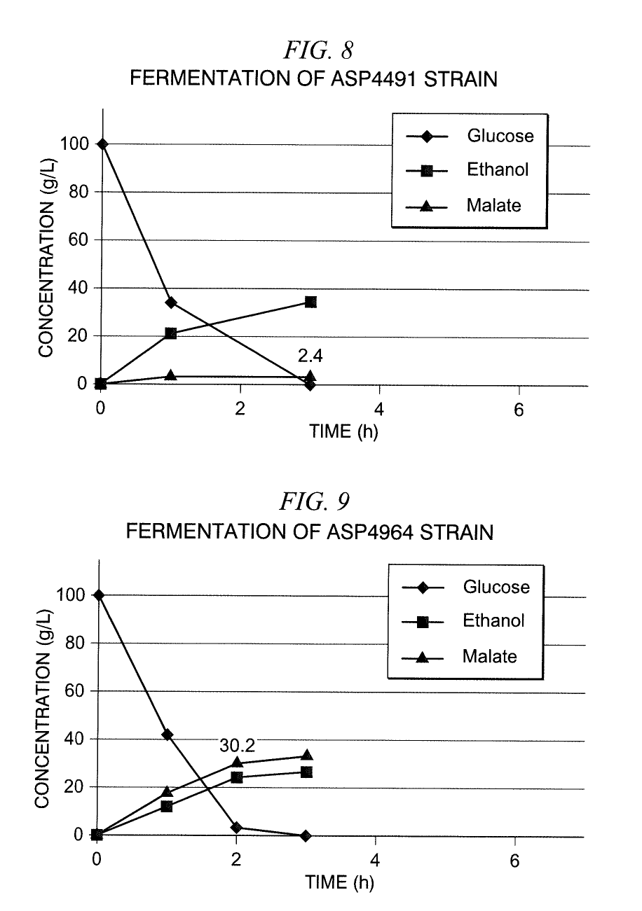

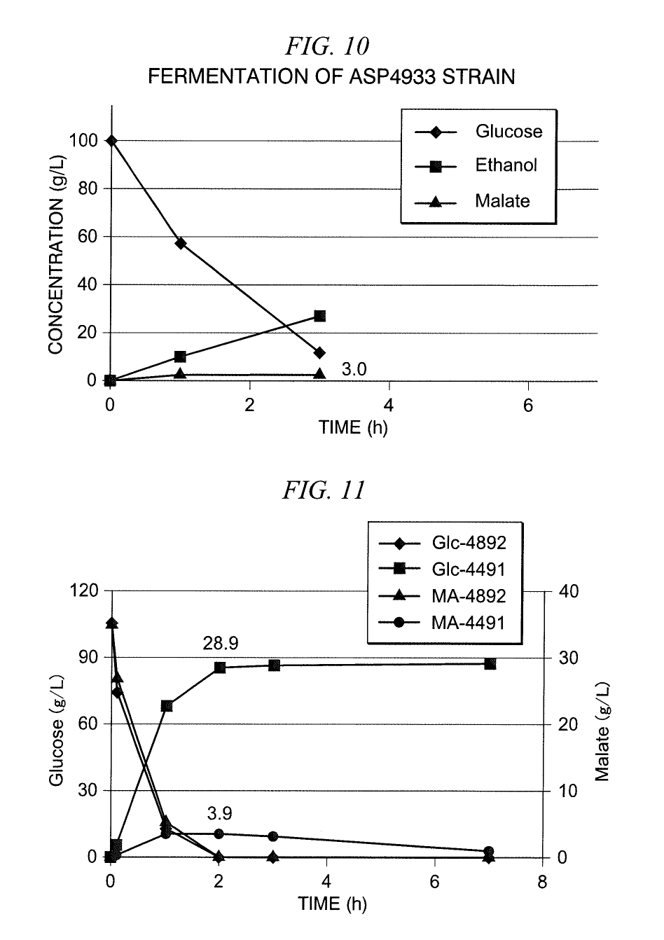

The measured results of the glucose concentration (g/L), the ethanol concentration (g/L), and a malic acid concentration (g/L) are shown in FIGS. 6 to 10. The results show that the production of malic acid was confirmed only in the ASP4964 strain (ΔPDC2, Δmae2, +ScePYC, +DacMDH).

<Measurement of Malic Acid Production Rate>

For the ASP4491 strain (ΔPDC2, +ScePYC, +DacMDH) and the ASP4892 strain (ΔPDC2, Δmae2, +ScePYC, +DacMDH), a malic acid production rate was measured.

Specifically, each strain was fermented in the same manner as in <Confirmation of malic acid producibility>, a sample was collected in time from the fermented liquid, and a glucose concentration and a malic acid concentration were measured. The measured results are shown in FIG. 11. In FIG. 11, “Glu-4892” and “Glu-4491” show a temporal change of a glucose concentration of the ASP4892 strain and the ASP4491 strain respectively, and “MA-4892” and “MA-4491” show a temporal change of a malic acid concentration of the ASP4892 strain and the ASP4491 strain respectively. As a result, it was confirmed that at a point in time when 1 hour elapsed from the beginning of fermentation, a malic acid concentration of the fermented liquid of the ASP4892 strain exceeded 20 g/L, and a malic acid production rate thereof was equal to or higher than 20 g/(L·h). Furthermore, at a point in time when 2 hours elapsed, a malic acid concentration of the fermented liquid became 28.9 g/L. In contrast, in the fermented liquid of the ASP4491 strain, a malic acid concentration was 3.9 g/L at a point in time when 2 hours elapsed.

### Example 5 Preparation of PCK Introduction Strain

Transformants of pombe were prepared (Table 47) into which PCK derived from Candida glabrata (CglPCK) (SEQ ID NO: 94), PCK derived from Citrobacter koseri (CkoPCK) (SEQ ID NO: 95), PCK derived from Cronobacter sakazakii (CsaPCK) (SEQ ID NO: 96), PCK derived from Debaryomyces hansenii (DhaPCK) (SEQ ID NO: 97), PCK derived from Escherichia fergusonii (EfePCK) (SEQ ID NO: 98), PCK derived from Edwardsiella tarda (EtaPCK) (SEQ ID NO: 99), PCK derived from Kluyveromyces lactic (KlaPCK) (SEQ ID NO: 100), PCK derived from Lodderomyces elongisporus (LelPCK) (SEQ ID NO: 101), PCK derived from Pectobacterium carotovorum (PcaPCK) (SEQ ID NO: 102), PCK derived from Photobacterium leiognathi (PlePCK) (SEQ ID NO: 103), PCK derived from Providencia rettgeri (PrePCK) (SEQ ID NO: 104), PCK derived from Saccharomyces cerevisiae (ScePCK) (SEQ ID NO: 105), PCK derived from Serratia odorifera (SodPCK) (SEQ ID NO: 106), PCK derived from Vibrio orientalis (VorPCK) (SEQ ID NO: 107), PCK derived from Vanderwaltozyma polyspora (VpoPCK) (SEQ ID NO: 108), PCK derived from Vibrio tubiashii (VtuPCK) (SEQ ID NO: 109), PCK derived from Yersinia bercovieri (YbePCK) (SEQ ID NO: 110), PCK derived from Yarrowia lipolytica (YliPCK) (SEQ ID NO: 111), PCK derived from Yersinia rohdei (YroPCK) (SEQ ID NO: 112), or PCK derived from Zygosaccharomyces rouxii (ZroPCK) (SEQ ID NO: 113) was introduced.

Specifically, the IGF836 strain (gene deletion strain of S. pombe) prepared in Example 1 was transformed according to the method of Bahler et al. (the journal of Yeast, 1998, Vol. 14, pp. 943-951) by using a digest of a restriction enzyme BsiWI of each monodentate integrative recombinant vector retaining each expression cassette of the PCK gene described in Table 47.

pSLh-CglPCK was prepared through the following process. First, by using total genomic DNA prepared by DNeasy (manufactured by QUIAGEN) from the culture of Candida glabrata as a template and using 2 kinds of synthetic oligo DNA (CglPCK-F and CglPCK-R, manufactured by Operon Biotechnologies) described in Table 27, an ORF fragment of a CglPCK gene was obtained by a PCR method using KOD-Dash (manufactured by TOYOBO CO., LTD.). The ORF fragment encoded CglPCK (SEQ ID NO: 94).

TABLE 27

| | Sequence | SEQ ID NO: |
|---|---|---|
| CglPCK-F | 5′-GACACTTTTTCAAAATGCCATCTAAATCTAGTGTTTCTGGG- | 114 |
| | 3′ | |
| CglPCK-R | 5′-GAAATCAACTTTTGTTCCAATTGTGGACCAGCAGCCAA-3′ | 115 |
| CglPCK | MPSKSSVSGNSVEERIRSELGLSKEVTLIRRNAPAAVLYQDALKEK | 94 |
| | KTVISSAGALIAYSGEKTGRSPKDKRIVEEETSRDNVWWGPVNKPC | |
| | SERTWAINRERAADYLRTRETLYVVDAFAGWDPKYRIKVRVVCARA | |
| | YHALFMTNMLIRPTEEELANFGEPDFTVWNAGQFPANARTQDMTSK | |
| | TTIEINFKAMEMVILGTEYAGEMKKGIFTVMFYLMPVHHNVLTLHS | |
| | SCNQGIKNGDVTLFFGLSGTGKTTLSADPHRLLIGDDEHCWSDEGV | |
| | FNIEGGCYAKCINLSREKEPEIFDAIRFGSVLENVIYDSESHEVDY | |
| | DDSSITENTRCAYPIDFIPSAKIPCLAPAHPKNIILLTCDASGVLP | |
| | PVSKLTPEQVMYHFISGYTSKMAGTEQGVTEPEPTFSSCFGQPFLS | |
| | LHPMRYATMLAEKMHEHSANAWLINTGWTGSSYVSGGKRCALKYTR | |
| | AILDAIHDGSLAKAEFESLPIFNLQVPKAVEGVPSELLNPARNWSE | |
| | GEAKYQSAVSKLAGLFVENFKTYQDKATSDVLAAGPQL* | |

By the same method as described above, a monodentate integrative recombinant vector pSLh-CkoPCK, a monodentate integrative recombinant vector pSLh-CsaPCK, a monodentate integrative recombinant vector pSLh-DhaPCK, a monodentate integrative recombinant vector pSLh-EfePCK, a monodentate integrative recombinant vector pSLh-EtaPCK, a monodentate integrative recombinant vector pSLh-KlaPCK, a monodentate integrative recombinant vector pSLh-LelPCK, a monodentate integrative recombinant vector pSLh-PcaPCK, a monodentate integrative recombinant vector pSLh-PlePCK, a monodentate integrative recombinant vector pSLh-PrePCK, a monodentate integrative recombinant vector pSLh-ScePCK, a monodentate integrative recombinant vector pSLh-SodPCK, a monodentate integrative recombinant vector pSLh-VorPCK, a monodentate integrative recombinant vector pSLh-VpoPCK, a monodentate integrative recombinant vector pSLh-VtuPCK, a monodentate integrative recombinant vector pSLh-YbePCK, a monodentate integrative recombinant vector pSMh-YliPCK, a monodentate integrative recombinant vector pSMh-YroPCK, and a monodentate integrative recombinant vector pSMh-ZroPCK were prepared. Primer sets using the respective vectors will be listed below.

TABLE 28

| | Sequence | SEQ ID NO: |
|---|---|---|
| CkoPCK-F | 5′-GACACTTTTTCAAAATGCGCGTTAACAGCTTAACCC-3′ | 116 |
| CkoPCK-R | 5′-GAAATCAACTTTTGTTCCAGCTTCGGCCCGGC-3′ | 117 |
| CkoPCK | MRVNSLTPQDLKAYGINDVQDIVYNPSYDLLYQEELDPTLEGYERG | 95 |
| | VLTNLGAVAVDTGIFTGRSPKDKYIVRDDTTRDTLWWSDKGKGKND | |
| | NKPLSQETWQHLKGLVTQQLSGKRLFIVDAFCGANADTRLSVRFIT | |
| | EVAWQAHFVKNMFIRPADEELVDFKPDFIVMNGAKCTNPQWKEQGL | |
| | NSENFVAFNLTERIQLIGGTWYGGEMKKGMFSVMNYLLPLKGIASM | |
| | HCSANVGEKGDVAVFFGLSGTGKTTLSTDPKRRLIGDDEHGWDDDG | |
| | VFNFEGGCYAKTIKLSKEAEPEIYNAIRRDALLENVTVREDGSIDF | |
| | DDGSKTENTRVSYPIYHIDNIVKPVSKAGHATKVIFLTADAFGVLP | |
| | PVSRLTADQTQYHFLSGFTAKLAGTERGVTEPTPTFSACFGAAFLS | |
| | LHPTQYAEVLVKRMQASGAQAYLVNTGWNGTGKRISIKDTRAIIDA | |
| | ILNGSLDNAETFNLPMFDLAIPTELPGVETRILDPRNTYASPEQWQ | |
| | EKATALAKLFVENFEKYTDTPAGEALVSAGPKL* | |

TABLE 29

| | Sequence | SEQ ID NO: |
|---|---|---|
| CsaPCK-F | 5′-GACACTTTTTCAAAATGCGAGTTACAGGCATAACCC-3′ | 118 |
| CsaPCK-R | 5′-GAAATCAACTTTTGTTCGCGCTGTGGGCCTGC-3′ | 119 |
| CsaPCK | MRVTGITPQDLKAYGINDVQEVVYNPDYDTLYREELDPSLEGYERG | 96 |
| | VLTDLGAVAVDTGIFTGRSPKDKYIVRDDTTRDTLWWSDNGKGKND | |
| | NKPLTPETWQHLKGLVTQQLSGKRLFIIDAFCGANPDSRLSVRFIT | |
| | EVAWQAHFVKNMFIRPSEDELEGFEPDFIVMNGAKCTNPDWQEQGL | |
| | NSENFVAFNLTERMQLIGGTWYGGEMKKGMFSIMNYLLPLKGIASM | |
| | HCSANVGEKGDVAVFFGLSGTGKTTLSTDPKRRLIGDDEHGWDDDG | |
| | VFNFEGGCYAKTIRLSEEAEPDIYHAIRRDALLENVTVRDDGSIDF | |
| | DDASKTENTRVSYPIYHIDNIVKPVSKAGHATKVIFLTADAFGVLP | |
| | PVSRLTASQTQYHFLSGFTAKLAGTERGVTEPTPTFSACFGAAFLM | |
| | LHPTQYSEVLVKRMQAAGAQAYLVNTGWNGTGKRISIKDTRAIIDA | |
| | ILNGSLDDAETFTLPLFNLAIPTSLPGVDERILDPRNTYASPEQWQ | |
| | EKAQQLAQLFISNFEKYTDTPAGAALVSAGPQR* | |

TABLE 30

| | Sequence | SEQ ID NO: |
|---|---|---|
| DhaPCK-F | 5′-GACACTTTTTCAAAATGACACCTCCTACTGCTGTTGA-3′ | 120 |
| DhaPCK-R | 5′-GAAATCAACTTTTGTTCAGCATCAGGACCAGCAGC-3′ | 121 |
| DhaPCK | MTPPTAVESSINFGGHPTIKSTAEPVVKQLSLADDTVIRHNAPPPT | 97 |
| | LYEDGLLEKGTVISSTGALMAYSGKKTGRSPKDKRIVDEETSTQNI | |
| | WWGPVNKQVDELTWKISRSRALDYLRTREKLFVVDAYAGWDPRYRL | |
| | KIRVICARAYHALFMTNMLIRPTEEELKNFGEPDFTIYNAGQFPAN | |
| | VHTKGMTSSTSVEINFKDMEMVILGTEYAGEMKKGIFTVMFYLMPI | |
| | KHKILTLHSSANQGEQKGDVTLFFGLSGTGKTTLSADPNRKLIGDD | |
| | EHCWSDNGVFNIEGGCYAKCLDLSAEKEPEIFNSIKFGAILENVVY | |
| | DQNSKVVDYADSTITENTRCAYPIDFIPSAKIPCLADEHPTNIVLL | |
| | TCDASGVLPPVSKLTNAQVMYHFISGYTSKMAGTEEGVTEPQATFS | |
| | ACFGQPFLVLHPMKYAQQLSDKISKHNANAWLLNTGWVGASAAQGG | |
| | NRCSLKYTRAILDAIHSGELSKVEFENFPTFNLNVPKSCPNVPSEI | |
| | LNPTKAWTEGDASFQKEIKSLAGKFAENFTKYADQATTEVKAAGPD | |
| | A* | |

TABLE 31

| | Sequence | SEQ ID NO: |
|---|---|---|
| EfePCK-F | 5′-GACACTTTTTCAAAATGCGCGTGAACAAAAGTTTAACC-3′ | 122 |
| EfePCK-R | 5′-GAAATCAACTTTTGTTCCATCTTCGGACCTGCTTCTACCA- | 123 |
| | 3′ | |
| EfePCK | MRVNKSLTPQDLMAYGINDVQDIVYNPSYDLLFQEELDPNLKGYER | 98 |
| | GVLTNLGAVAVDTGVFTGRSPKDKYIVRDDTTRDTFWWADKGKGKN | |
| | DNKPLSPETWQHLKGLVTNQLSGKRLFVVDAFCGANADTRLSVRFI | |
| | TEVAWQAHFVKNMFIRPTDEELVDFEPDFIVMNGAKCTNPQWKEQG | |
| | LNSENFVAFNLTERMQLIGGTWYGGEMKKGMFSMMNYLLPLKGIAS | |
| | MHCSANVGEKGDVAVFFGLSGTGKTTLSTDPKRRLIGDDEHGWDDD | |
| | GVFNFEGGCYAKTIKLSKEAEPEIYNAIRRDALLENVTVREDGTID | |
| | FDDGSKTENTRVSYPIYHIDNIVKPVSKAGHATKVIFLTADAFGVL | |
| | PPVSRLTADQTQYHFLSGFTAKLAGTERGITEPTPTFSACFGAAFL | |
| | SLHPTQYAEVLVKRMQAAGAQAYLVNTGWNGTGKRISIKDTRAIID | |
| | AILNGSLDNAETFTLPMFNLAIPTELPGVDTEILDPRNTYASPEQW | |
| | QEKAETLAKLFIDNFDKYTDTPAGAALVEAGPKM* | |

TABLE 32

| | Sequence | SEQ ID NO: |
|---|---|---|
| EtaPCK-F | 5′-GACACTTTTTCAAAATGCATGCCACTGGCTTAACT-3′ | 124 |
| EtaPCK-R | 5′-GAAATCAACTTTTGTTCACGTTGTGGGCCGGCA-3′ | 125 |
| EtaPCK | MHATGLTAADLASYGISDVTEIVYNPDYDLLFKEETAPGLQGYERG | 99 |
| | TVTTLGAVAVDTGIFTGRSPKDKYLVRDDTTRDTVWWSDQGKGKND | |
| | NHPLSPEIWQHLKGLVTRQLSNKRLFVVDAYCGANPDSRLKVRFIT | |
| | EVAWQAHFVKNMFIRPTEDELAGFEPDFIVMNGAKCTNPQWQEQGL | |
| | NSENFVAFNLTERIQLIGGTWYGGEMKKGMFSIMNYLLPLKGIASM | |
| | KCSANVGEKGDVAVFFGLSGTGKTTLSTDPKRRLIGDDEHGWDDDG | |
| | VFNFEGGCYAKTIKLSAEAEPDIYHAITRDALLENVTVRADGSVDF | |
| | DDGSKTENTRVSYPIYHIKNIVKPVSKAGHAKKVIFLTADAFGVLP | |
| | PVSRLTADQTQYHFLSGFTAKLAGTERGVTEPTPTFSACFGAAFLT | |
| | LHPTQYAEVLVKRMQAAGAQAYLVNTGWNGSGKRISIKDTRAIIDA | |
| | ILNGDIDKEETFTLPIFNLAVPTALPGVNPAILDPRDTYASPEMWQ | |
| | EKADDLAQRFITNFDKYTDTPAGAALVHAGPQR* | |

TABLE 33

| | |
|---|---|
| Sequence | SEQ ID NO: |

| | | |
|---|---|---|
| KlaPCK-F | 5′-GACACTTTTTCAAAATGTCACCAACTAAGACACAAAATTCTG-3′ | 126 |
| KlaPCK-R | 5′-GAAATCAACTTTTGTTCTAATTGCGGACCAGCAGCTAAG-3′ | 127 |
| KlaPCK | MSPTKTQNSAEENIRAELGLSAEVVTIRRNAPAALLYEDALKERDT | 100 |
| | AISNAGALIAYSGDKTGRSPRDKRIVEEETSKDNVWWGPVNKPCSE | |
| | RTWEINRERAADYLRTRDHIYIVDAYAGWDPRYRIKVRVVCARAYH | |
| | ALFMTNMLIRPSKEELENFGEPDFTVWNAGQFPANTHTSGMTSKTT | |
| | VEINFKQMEMVILGTEYAGEMKKGIFTVMFYLMPVNHNVLTLHSSA | |
| | NQGIQDNDVTLFFGLSGTGKTTLSADPHRLLIGDDEHCWSDHGVFN | |
| | IEGGCYAKCLGLSAEKEPEIFNAIKFGSVLENIIYDPNTREVDYED | |
| | STITENTRCAYPIEYIPSAKIPCLADHHPKNIVLLTCDASGVLPPV | |
| | SKLTPDQVMYHFISGYTSKMAGTEQGVTEPEATFSSCFGQPFLSLH | |
| | PMKYATMLAEKMAEHNANAWLINTGWTGSSYVAGGKRCPLKYTRAI | |
| | LDAIHDGSLAKEEYEVLPIFNLQIPKAVGDKVPASLLNPSKNWAEG | |
| | EAKYTSNVKSLANLFVENFKTYQDKATEQVLAAGPQL* | |

TABLE 34

| | Sequence | SEQ ID NO: |
|---|---|---|
| LelPCK-F | 5′-GACACTTTTTCAAAATGGCACCCCCAGCAGTA-3′ | 128 |
| LelPCK-R | 5′-GAAATCAACTTTTGTTCAACATCGGGTCCAGCAGCT-3′ | 129 |
| LelPCK | MAPPAVESTINFEGHPSIXTTVDPLVKKLNLKGDTVIRHNAPPPTL | 101 |
| | YEDGLLEKGTTISSTGALMAYSGEKTGRSPKDKRIVDEETSSQHIW | |
| | WGPVNKQVDELTWKISRSRALDYLRTREKLFVVDAFAGWDPKYRIK | |
| | VRIICARAYHALFMTNMLIRPTKEELENFGEPDFTIYNAGQFPANV | |
| | HTKGMTSSTSVEINFKDMEMVILGTEYAGEMKKGIFTVMFYLMPIK | |
| | NKVLTLHSSANEGESGDVTLFFGLSGTGKTTLSADPNRKLIGDDEH | |
| | CWSDHGVFNIEGGCYAKCLDLSAEKEPEIFNSIRFGSVLENVVYDP | |
| | ATKVVDYEDSSITENTRCSYFIEYIPSLKIPCVGGHPKNIVLLTCD | |
| | ASGVLPPVSKLTPAQVMYHFISGYTSKMAGTEMGITEPTPAFSACF | |
| | GQPFLVLHPMKYAQQLAEKIEQHKANAYLLNTGWSGVGAAKGGKRC | |
| | PLKYTRAILDAIHSGELDSADTQVFSTFNLHVPKEIKGVPLEVLHP | |
| | EVDKKEVAALAAKFAENFVKYADQATAEVKAAGPDV* | |

TABLE 35

| | Sequence | SEQ ID NO: |
|---|---|---|
| PcaPCK-F | 5′-GACACTTTTTCAAAATGCAGATCAACGGTATTACCCC-3′ | 130 |
| PcaPCK-R | 5′-GAAATCAACTTTTGTTCGCGCTTCGGCCCTGC-3′ | 131 |
| PcaPCK | MAPPAVESTINFEGHPSIKTTVDPLVKKLNLKGDTVIRHNAPPPTL | 102 |
| | YEDGLLEKGTTISSTGALMAYSGEKTGRSPKDKRIVDEETSSQHIM | |
| | WGPVNKQVDELTWKISRSRALDYLRTREKLFVVDAFAGWDPKYRIK | |
| | VRIICARAYHALFMTNMLIRPTKEELENFGEPDFTIYNAGQFPANV | |
| | HTKGMTSSTSVEINFKDMEMVILGTEYAGEMKKGIFTVMFYLMPIK | |
| | NKVLTLHSSANEGESGDVTLFFGLSGTGKTTLSADPNRKLIGDDEH | |
| | CWSDHGVFNIEGGCYAKCLDLSAEKEPEIFNSIRFGSVLENVVYDP | |
| | ATKVVDYEDSSITENTRCSYPIEYIPSLKIPCVGGHPKNIVLLTCD | |
| | ASGVLPPVSKLTPAQVMYHFISGYTSKMAGTEMGITEPTPAFSACF | |
| | GQPFLVLHPMKYAQQLAEKIEQHKANAYLLNTGWSGVGAAKGGKRC | |
| | PLKYTRAILDAIHSGELDSADTQVFSTFNLHVPKEIKGVPLEVLHP | |
| | EVDKKEVAALAAKFAENETKYADQATAEVKAAGPDV* | |

TABLE 36

| | Sequence | SEQ ID NO: |
|---|---|---|
| PlePCK-F | 5′-GACACTTTTTCAAAATGAGCACGATGTGTGTCGATAATAA- | 132 |
| | 3′ | |
| PlePCK-R | 5′-GAAATCAACTTTTGTTCGTCTAACTGTGGTCCGGCTTTAAC- | 133 |
| | 3′ | |
| PlePCK | MSTMCVDNKVATNMDLSQYGILNVTDVIRNPSYEMLFEEETKAELK | 103 |
| | GFEKGIVTELGAVAVDTGIFTGRSPQDKFIVKDDTTKDTLWWSDQG | |
| | KNDNKALSQGAWNDLKSLVTTQLSNKRLFVVDGYCGANPDTRLCIR | |
| | VITEVAWQAHFVENMFIRPTEAELDTFEPDFVIMNGSKCTNPKWQE | |
| | HGMNSENFTVFNLSEKMQLIGGTWYGGEMKKGMFAMMNYFLPLKDI | |
| | ASMHCSANMGEAGDVAIFFGLSGTGKTTLSTDPKRALIGDDEHGKN | |
| | DNGVFNFEGGCYAKTINLSKEAEPDIYNAIRRDALLENVTVRGDGS | |
| | IDFNDNSKTENTRVSYPIYHIDNIVKPVSKGGHANKVIFLSADAFG | |
| | VLPPVSKLTPEQTKYHFLSGFTAKLAGTERGITEPTPTFSACFGNA | |
| | FLTLHPTQYAEVLVKRMEAAGAEAYLVNTGWNGTGKRISIQDTRGI | |
| | IDAILDGSIDTAPTKQIPIFNLTVPTALPGVDPEILDPRDTYTDPL | |
| | QWESKATDLAGRFIANFDKYTDTTEGKSLVKAGPQLD* | |

TABLE 37

| | Sequence | SEQ ID NO: |
|---|---|---|
| PrePCK-F | 5′-GACACTTTTTCAAAATGAGCGCTAAAAGCATTACCCT-3′ | 134 |
| PrePCK-R | 5′-GAAATCAACTTTTGTTCCAGTTTTGGCCCTGCTTTCAC-3′ | 135 |
| PrePCK | MSAKSITLKELEKYGIHDVTEVVYNPSYELLFTEETKPGLEGYERG | 104 |
| | TVTTLGAVAVDTGIFTGRSPKDKYIVRDDVTRDTVWWADQGKGKND | |
| | NKPMSQEVWADLKHLVTEQLSGKRLFIIDAFCGANADTRLKVRFIT | |
| | EVAWQAHFVKNMFIRPSDEELVGFEPDFIVMNGAKCTNPNWKAQGL | |
| | NSENFVAFNLTERMQLIGGSWYGGEMKKGMFSMMNYLLPLKGIASM | |
| | HCSANVGEKGDVAIFFGLSGTGKTTLSTDPKRKLIGDDEHGWDDDG | |
| | VFNFEGGCYAKTINLSKEAEPDIYGAIKRDALLENVMVLADGSVDF | |
| | NDGSKTENTRVSYPIYHIENIVKPVSKAGHATKVIFLTADAFGVLP | |
| | PVSRLTPEQTQYHFLSGFTAKLAGTERGVTEPTPTFSACFGAAFLS | |
| | LHPTQYAEVLVKRMEAAGAKAYLVNTGWNGTGKRISIKDTRAIIDA | |
| | ILSGDIEKADMIKLPVFDLEIPTALPGVDTNILDPRNTYADKAQWD | |
| | EKAQDLAERFVNNFDKYTDTPAGAALVKAGPKL* | |

TABLE 38

| | Sequence | SEQ ID NO: |
|---|---|---|
| ScePCK-F | 5′-GACACTTTTTCAAAATGTCCCCTTCTAAAATGAATGCTACAG- | 136 |
| | 3′ | |
| ScePCK-R | 5′-GAAATCAACTTTTGTTCCTCGAATTGAGGACCAGCGGC-3′ | 137 |
| ScePCK | MSPSKMNATVGSTSEVEQKIRQELALSDEVTTIRRNAPAAVLYEDG | 105 |
| | LKENKTVISSSGALIAYSGVKTGRSPKDKRIVEEPTSKDEIWWGPV | |
| | NKPCSERTWSINRERAADYLRTRDHIYIVDAFAGWDPKYRIKVRVV | |
| | CARAYHALFMTNMLIRPTEEELAHFGEPDFTVWNAGQFPANLHTQD | |
| | MSSKSTIEINFKAMEMIILGTEYAGEMKKGIFTVMFYLMPVHHNVL | |
| | TLHSSANQGIQNGDVTLFTGLSGTGKTTLSADPHRLLIGDDEHCWS | |
| | DHGVFNIEGGCYAKCINLSAEKEPEIFDAIKFGSVLENVIYDEKSH | |
| | VVDYDDSSITENTRCAYPIDYIPSAKIPCLADSHPKNIILLTCDAS | |
| | GVLPPVSKLTPEQVMYHFISGYTSKMAGTEQGVTEPEPTFSSCFGQ | |
| | PFLALHPIRYATMLATKMSQHKANAYLINTGWTGSSYVSGGKRCPL | |
| | KYTRAILDSIHDGSLANETYETLPIFNLQVPTKVNGVPAELLNPAK | |
| | NWSQGESKYRGAVTNLANLFVQNFKIYQDRATPDVLAAGPQFE* | |

TABLE 39

| | Sequence | SEQ ID NO: |
|---|---|---|
| SodPCK-F | 5′-GACACTTTTTCAAAATGCGTGCTAAAGGTATCACCC-3′ | 138 |
| SodPCK-R | 5′-GAAATCAACTTTTGTTCCAGCTTCGGACCGGCG-3′ | 139 |
| SodPCK | MRAKGITHQDLAAYGIHDIGEIIHNPSYDLLFEEETDPSLTGYERG | 106 |
| | VVTKLGAVAVDTGIFTGRSPKDKYIVRDDTTRDTVWWADQGKGKND | |
| | NKPMSPEVWASLKTLVTEQLSGKRLFVVDTFCGANADSRLKVRFIT | |
| | EVAWQAHFVKNMFIRPSDDELADFEPDFVVMNGAKCTNPDWQQQGL | |
| | NSENFVAFNLTERMQLIGGTWYGGEMKKGMFSIMNYLLPLKGIASM | |
| | HCSANVGEQGDVAIFFGLSGTGKTTLSTDPKRQLIGDDEHGWDDDG | |
| | VFNEEGGCYAKTIKLSEQAEPDIYRAIKRDALLENVTVLADGSVDF | |
| | NDGSKTENTRVSYPIYHIQNIVKPVSKAGHATKVIFLTADAFGVLP | |
| | PVSRLTADQTQYHFLSGFTAKLAGTERGVTEPTPTFSACFGAAFLS | |
| | LHPTQYAEVLVKRMQAAGAQAYLVNTGWNGSGKRISIQDTRGIIDA | |
| | ILSGEIDNAETITLPIFDLAVPTALPGVNPAILDPRATYQSEAQWE | |
| | DKAKDLAQRFITNFDKYTDTPAGEALIGAGPKL* | |

TABLE 40

| | Sequence | SEQ ID NO: |
|---|---|---|
| VorPCK-F | 5′-GACACTTTTTCAAAATGACCGTTATGGAACATACTAAGGC- | 140 |
| | 3′ | |
| VorPCK-R | 5′-GAAATCAACTTTTGTTCATCTAGCTGAGGACCTGCAGC-3′ | 141 |
| VorPCK | MTVMEHTKAATIDLTKHGLHNVKEVIRNPSYEMLFEEETRADLEGY | 107 |
| | EKGVVTELGAVAVDTGIFTGRSPKDKYIVKDATTEEHMWWTSDAVK | |
| | NDNKPIDQAVWNDLKAQVTEQLSGKRVFVIDGYCGANPDTRLSIRV | |
| | ITEVAWQAHFVKNMFIRPSEEELTTFEPDFVVMNGAKCTNQKWEQH | |
| | GLNSENFTVFNLTERMQLIGGTWYGGEMKKGMFANMNYFLPLKDIA | |
| | SMHCSANKCKENGDVAIFFGLSGTGKTTLSTDPKRELIGDDEHGWD | |
| | DDGVFNFEGGCYAKTIKLSKEAEPDIYNAIRRDALLENVTVRNDGS | |
| | IDFDDGSKTENTRVSYPIEHIENIVKPVSRAGHANKVIFLSADAFG | |
| | VLPPVSKLTPEQTKYHFLSGFTAKLAGTERGITEPTPTFSACFGAA | |
| | FLTLHPTKYAEVLVKRMEAAGAEAYLVNTGWNGSGKRISIQDTRGI | |
| | IDAILDGSIEEAETKHIPIFNLEVPTALHDVDPSILDPRETYTDPL | |
| | QWESKAKDLASRFINNFDKYTDNDEGKSLVAAGPQLD* | |

TABLE 41

| | Sequence | SEQ ID NO: |
|---|---|---|
| VpoPCK-F | 5′-GACACTTTTTCAAAATGGCTCCAACTAAAATAGAAGATTACT | 142 |
| | CT-3′ | |
| VpoPCK-R | 5′-GAAATCAACTTTTGTTCTAGGGCTGGACCAGCTGC-3′ | 143 |
| VpoPCK | MAPTKIEDYSLNERENIIKNLKSNSNVKLDNVMKCLYNKDITIKRN | 108 |
| | PPIAELYEDGLKEDSTTLSSTGALMAYSGVKTGRSPKDKRIVEEPS | |
| | SKDNIWWGPVNKPCSELTWSINRERAIDYLNTRKILYVIDAYAGWD | |
| | ANYRIKIRVVCARPYHALFMNNMLIRPTTEELENFGEPDFTIWNAG | |
| | SFYANSLTKDMTSKTSIEINFKSMEMVILGTEYAGEMKKGIFTVMF | |
| | YLMPVYHNVLTLHSSANQGVNNGDVTLFFGLSGTGKTTLSADPHRE | |
| | LIGDDEHCWSDHGVFNIEGGCYAKCIGLTEEKEPEIFNAIKFGSVL | |
| | ENCVYDENTHVVDYDDSRITENTRCAYPIDFIESAKIPCLTDKHPT | |
| | NLILLTCDASGVLPPVSKLTPEQVMYHFISGYTSKMAGTEQGVTEP | |
| | EPTFSACFGQPFLALHPMKYAQMLADKMNHHKASAWLINTGWTGAS | |
| | YTAGGKRCPLKYTRAILDAIHNGSLANEEYETLPVFNLPIPKNVEN | |
| | VPSELLNPSKTWSDPDVTYSQAVKKLGSLFVQNFTIYQDQATDDVI | |
| | ARGPAL* | |

TABLE 42

| | Sequence | SEQ ID NO: |
|---|---|---|
| VtuPCK-F | 5′-GACACTTTTTCAAAATGACCGTTATGGAACATACAAAGGC- | 144 |
| | 3′ | |
| VtuPCK-R | 5′-GAAATCAACTTTTGTTCATCTAGCTGAGGTCCAGCCG-3′ | 145 |
| VtuPCK | MTVMEHTKAATIDLTKHGLQNVKEVIRNPSYEMLFEEETRADLEGY | 109 |
| | EKGVVTELGAVAVDTGIFTGRSPKDKYIVKDATTEEHMWWTSDAVK | |
| | NDNKPIDQAVWNDLKQLVTNQLSGKRVFVIDGYCGANPDTRLSIRV | |
| | ITEVAWQAHFVKNMFIRPSEKELATFEPDFVVMNGAKCTNQKWEEH | |
| | GLNSENFTVFNLTERMQLIGGTWYGGEMKKGMFAMMNYFLPLKDIA | |
| | SMHCSANKCKENGDVAVFFGLSGTGKTTLSTDPKRELIGDDEHGWD | |
| | DDGIFNFEGGCYAKTIKLSKEAEPDIYNAIRRDALLENVTVRNDGS | |
| | IDFDDGSKTENTRVSYPIEHIENIVKPVSKAGHANKVIFLSADAFG | |
| | VLPPVSKLTPEQTKYHFLSGFTAKLAGTERGITEPTPTFSACFGAA | |
| | FLTLHPTKYAEVLVKRMEAAGAEAYLVNTGWNGSGKRISIQDTRGI | |
| | IDAILDGSIENAETKHIPIFNLEVPTALHDVDPAILDPRDTYTDPL | |
| | QWESKAKDLAERFINNFDKYTDNDEGKSLVAAGPQLD* | |

TABLE 43

| | Sequence | SEQ ID NO: |
|---|---|---|
| VbePCK-F | 5′-GACACTTTTTCAAAATGAGTGTTAAAGGAATTACCCCGC- | 146 |
| | 3′ | |
| VbePCK-R | 5′-GAAATCAACTTTTGTTCGATCTTCGGCCCTGCGCT-3′ | 147 |
| VbePCK | MSVKGITPQELAAYGIHNVSEIVYNPSYDLLFQEETKPTLEGYERG | 110 |
| | TLTTTGAIAVDTGIFTGRSPKDKYIVRDAITQDTVWWADQGKGKND | |
| | NKPLSQETWSHLKGLVTEQLSGKRLFVVDTFCGANADSRLQVRFVT | |
| | EVAWQAHFVKNMFIRPTDEELADFEPDFIVMNGAKCTNPNWKEQGL | |
| | NSENFVAFNLTERMQLIGGTWYGGEMKKGMFSMMNYLLPLKGIASM | |
| | HCSANVGEKGDVAIFFGLSGTGKTTLSTDPKRKLIGDDEHGWDDDG | |
| | VFNFEGGCYAKTIKLSEEAEPDIYHAIRRDALLENVVVLPDGTVDF | |
| | NDSSKTENTRVSYPIYHIENIVKPVSKAGHATKVIFLTADAFGVLP | |
| | PVSRLTANQTQYHFLSGFTAKLAGTERGVTEPTPTFSACFGAAFLS | |
| | LHPTQYAEVLVKRMQAVGAQAYLVNTGWNGTGKRISIKDTRAIIDA | |
| | ILNGEIDKAETFTLPIFDLAVPMALPGVDPAILDPRDTYADIAQWQ | |
| | EKAEDLAKRRFTTNFDKYTDTPAGAALMSAGPKI* | |

TABLE 44

| | Sequence | SEQ ID NO: |
|---|---|---|
| YliPCK-F | 5′-GACACTTTTTCAAAATGTCTCCCCCCGTCAACC-3′ | 148 |
| YliPCK-R | 5′-GAAATCAACTTTTGTTCAGCTTAGGGCCGGCA-3′ | 149 |
| YliPCK | MSPPVNQTQLHPKGVEPTPAAEEIEKELYDIAHIEYERVNIDHNPS | 111 |
| | VASLYEDALVQESGSAINSTGALVASSGKKTGRSPKDKRIVEEPGS | |
| | VEHIWWGPVNKKLSEQSWLINRERAVDYLNTRKRIYVVDAYAGWDP | |
| | KYRIKVRIVCARAYHALFMRNMLIRPTAEELKNFGKPDFTIYNAGA | |
| | FPANRYTSGMTSNTSIDINFKSMEMLILGTEYAGEMKKGIFTVMFY | |
| | LMPVKHQVLTLHSSANEGKDGDVTLFFGLSGTGKTTLSADPNRLLI | |
| | GDDEHCWSDTGVFNIEGGCYAKCLGLSREKEPDIFDAIKFGSVLEN | |
| | VVFDPITREVDYDDATLTENTRCSYPIEYIPNAKLPCVTESHPTNI | |
| | ILLTCDARGVIPPISKLTNEQVMYHFISGYTSKMAGTEEGVTEPEA | |
| | TFSACFGQPFLVLHPMQYAKMLSDKMTQHNANAWLLNTGWTGQSYT | |
| | NGGKRCPLKYTRSILDAIHSGELAKVEYENFDVFNLQVPKTAPNVP | |
| | SEILNPARAWSGGKEQFTEEVTKLGKLFNENFEKYKDMAIPEVIAA | |
| | GPKA* | |

TABLE 45

| | Sequence | SEQ ID NO: |
|---|---|---|
| YroPCK-F | 5′-GACACTTTTTCAAAATGAGTGTTAAAGGAATTACCCCGC- | 150 |
| | 3′ | |
| YroPCK-R | 5′-GAAATCAACTTTTGTTCGACTTTTGGCCCAGCACTG-3′ | 151 |
| YroPCK | MSVKGITPQELAAYGIHNVSEIVYNPSYDLLFQEETKPTLEGYERG | 112 |
| | TLTTTGAIAVDTGIFTGRSPKDKYIVRDDITRDTVWWADQGKGKND | |
| | NKPLSQETWAHLKGLNTQQLSGKRLFVVDTFCGANADTRLQVRFVT | |
| | EVAWQAHFVKNMFIRPTDEELAHFEPDFIVMNGAKCTNPNWKEQGL | |
| | NSENFVAFNLTERMQLIGGTWYGGEMKKGMFSMMNYLLPLKGIASM | |
| | HCSANVGEKGDVAIFFGLSGTGKTTLSTDPKRKLIGDDEHGWDDDG | |
| | VFNFEGGCYAKTIKLSEEAEPDIYHAIKRDALLENVVVLPDGTVDF | |
| | NDGSKTENTRVSYPIYHIENIVKPVSKAGHATKVIFLTADAFGVLP | |
| | PVSRLTASQTQYHFLSGFTAKLAGTERGVTEPTPTFSACFGAAFLS | |
| | LHPTQYAEVLLKRMQAVGAQAYLVNTGWNGTGKRISIKDTRGIIDA | |
| | ILNGEIDKAETFTLPIFDLAVPMALPGVNPDILDPRDTYADVAQWQ | |
| | EKAEDLAKRFTTNFDKYTDTPAGAALVSAGPKV* | |

TABLE 46

| | Sequence | SEQ ID NO: |
|---|---|---|
| ZroPCK-F | 5′-GACACTTTTTCAAAATGTCGCCCTCCAAAGTTCC-3′ | 152 |
| ZroPCK-R | 5′-GAAATCAACTTTTGTTCGAATTGAGGACCAGCTGCAAGAA- | 153 |
| | 3′ | |
| ZroPCK | MSPSKVPVPITDTAEVEEQIRKELALGSEVVTIRHNAPAATLYEDG | 113 |
| | LKEKKTAVVSSGALAAYSGSKTGRSPKDKRIIEEETSKNEVWWGPV | |
| | NKPASEKTWSINRERAADYLRTRDHLYIIDAYAGWDPRYRIKVRVI | |
| | CARAYHALFMTNMLIRPSPEELANFGEPDFTVWNAGQFPANRNTEG | |
| | MTSKTTVEINFKAMEMIILGTEYAGEMKKGIFTVMFYLMPVHHNVL | |
| | TLHSSANQGIERGDVTLFFGLSGTGKTTLSADPHRALIGDDEHCWS | |
| | DHGVFNIEGGCYAKCIDLTAEKEPEIYNAIKFGSVLENVVFDASNR | |
| | EVDYTDNSITENTRCAYPIDYIPSAKIPCLADKHPSNIVLLTCDAS | |
| | GVLPPVSKLTPEQVMYHFISGYTSKMAGTEQGVTEPETTFSSCFGQ | |
| | PFLALHPMRYATMLAKKMHQHNANAWLVNTGWTGSSYTAGGKRCPL | |
| | KYTRAILDSIHDGSLSKADYETLPIFNLHTPAKVNGVPDSLLNPAK | |
| | SWVEGPTKYNSAVKTLATKFTENFKIYQDQATPDVLAAGPQF* | |

TABLE 47

| | | Name of introduced |
|---|---|---|
| Name of transformant strain | Name of host strain | foreign genes |
| 22 | IGF836 | CglPCK |
| 23 | IGF836 | CkoPCK |
| 24 | IGF836 | CsaPCK |
| 25 | IGF836 | DhaPCK |
| 26 | IGF836 | EfePCK |
| 27 | IGF836 | EtaPCK |
| 28 | IGF836 | KlaPCK |
| 29 | IGF836 | LelPCK |
| 30 | IGF836 | PcaPCK |
| 31 | IGF836 | PlePCK |
| 32 | IGF836 | PrePCK |
| 33 | IGF836 | ScePCK |
| 34 | IGF836 | SodPCK |
| 35 | IGF836 | VorPCK |
| 36 | IGF836 | VpoPCK |
| 37 | IGF836 | VtuPCK |
| 38 | IGF836 | YbePCK |
| 39 | IGF836 | YliPCK |
| 40 | IGF836 | YroPCK |
| 41 | IGF836 | ZroPCK |

<Confirmation of PCK Expression>

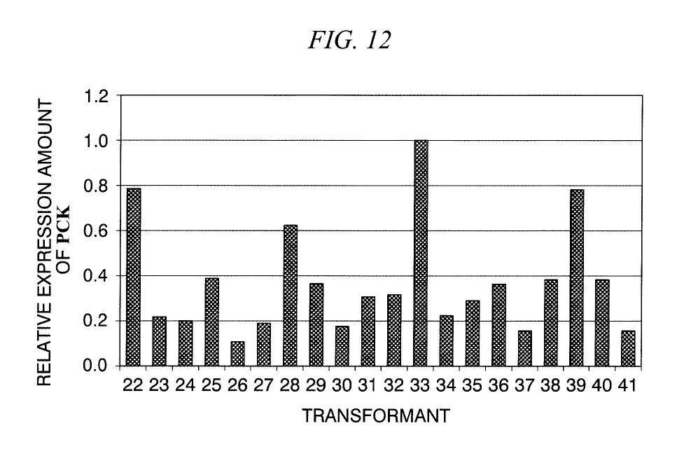

For 20 kinds of transformant into which only the PCK gene was introduced, an expression amount of PCK was confirmed. Specifically, each of the transformants was shake-cultured for 44 hours in 20 mL of a YPD6 medium, and the grown microbial cells were then collected and pulverized using a multibead shocker. The obtained microbial cell lysate was purified according to a protocol by using ANTI-FLAG (registered trademark) M1 Agarose Affinity Gel (manufactured by Sigma-Aldrich Co. LLC.). SDS-PAGE was performed using a purified enzyme liquid, and the detected band was digitized using an image analysis device LAS 4000, thereby calculating a relative expression amount. The results of the PCK expression amount (relative value) calculated for each of the transformants are shown in FIG. 12. The results show that the expression of PCK was confirmed in all of the transformants.

### Example 6 Preparation of EcoPCK Introduction Strain, GglPYC Introduction Strain, and EcoMDH Introduction Strain

Transformants of pombe were prepared into which PCK derived from Escherichia coli (EcoPCK) (SEQ ID NO: 154), PYC derived from Gallus gallus (GglPYC) (SEQ ID NO: 155), and MDH derived from Escherichia coli (EcoMDH) (SEQ ID NO: 156) were introduced.

Specifically, the IGF836 strain (gene deletion strain of S. pombe) prepared in Example 1 was transformed according to the method of Bahler et al. (the journal of Yeast, 1998, Vol. 14, pp. 943-951) by using a digest of a restriction enzyme BsiWI of a monodentate integrative recombinant vector pSNh-EcoPCK retaining an EcoPCK gene expression cassette, a monodentate integrative recombinant vector pSLh-GglPYC retaining a GglPYC gene expression cassette, or a monodentate integrative recombinant vector pSMh-EcoMDH retaining an EcoMDH gene expression cassette.

pSNh-EcoPCK was prepared through the following process. First, by using total genomic DNA prepared by DNeasy (manufactured by QUIAGEN) from the culture of Escherichia coli as a template and using 2 kinds of synthetic oligo DNA (EcoPCK-F and EcoPCK-R, manufactured by Operon Biotechnologies) described in Table 48, an ORF fragment of an EcoPCK gene was obtained by a PCR method using KOD-Dash (manufactured by TOYOBO CO., LTD.). The ORF fragment encoded EcoPCK (SEQ ID NO: 154).

TABLE 48

| | | SEQ |
|---|---|---|
| | | ID |
| | Sequence | NO: |
| EcoPCK-F | 5′-GACACTTTTTCAAAATGCGCGTTAACAATGGTTTG | 157 |
| | ACC-3′ | |
| EcoPCK-R | 5′-GAAATCAACTTTTGTTCCAGCTTCGGACCAGC-3′ | 158 |
| EcoPCK | MRVNNGLTPQELEAYGISDVHDIVYNPSYDLLYQEEL | 154 |
| | DPSLTGYERGVLTNLGAVAVDTGIFTGRSPKDKYIVRD | |
| | DTTRDTFWWADKGKGKNDNKPLSPETWQHLKGLVT | |
| | KQLSGKRLFVVDAFCGANPDTRLSVRFITEVAWQAHF | |
| | VKNMFIRPSDEELAGFKPDFIVMNGAKCTNPQWKEQ | |
| | GLNSENFVAFNLTERMQLIGGTWYGGEMKKGMFSM | |
| | MNYLLPLKGIASMHCSANVGEKGDVAVFFGLSGTGK | |
| | TTLSTDPKRRLIGDDEHGWDDDGVFNFEGGCYAKTIK | |
| | LSKEAEPEIYNAIRRDALLENVTVREDGTIDFDDGSKT | |
| | ENTRVSYPIYHIDNIVKPVSKAGHATKVIFLTADAFGV | |
| | LPPVSRLTADQTQYHFLSGFTAKLAGTERGITEPTPTF | |
| | SACFGAAFLSLHPIQYAEVLVKRMQAAGAQAYLVNTG | |
| | WNGTGKRISIKDTRAIIDAILNGSLDNAETFTLPMFNL | |
| | AIPTELPGVDTKILDPRNTYASPEQWQEKAETLAKLFI | |
| | DNFDKYTDTPAGAALVAAGPKL | |

By using an In-Fusion (registered trademark) HD Cloning Kit (manufactured by Clontech Laboratories, Inc.), the obtained amplified fragment was incorporated into pSNh, thereby preparing pSNh-EcoPCK. The In-Fusion method was performed according to the manual included in the kit. That is, the obtained PCR product was purified using a spin column, added to an In-Fusion reaction solution together with pSNh, and reacted for 15 minutes at 50° C.

By the same method as described above, a monodentate integrative recombinant vector pSLh-GalPYC and a monodentate integrative recombinant vector pSMh-EcoMDH were prepared. Primer sets using the respective vectors will be listed below.

TABLE 49

| | | SEQ |
|---|---|---|
| | | ID |
| | Sequence | NO: |
| GglPYC-F | 5′-CATCTTTTTCAAACATGTCGCAGCTGTGTGTCCC- | 159 |
| | 3′ | |
| GglPYC-R | 5′-ATCATCATCATCCTTGTAATCCTCGATCTCGGCGAT | 160 |
| | G-3′ | |
| GglPYC | MSQLCVPRGGRALLGAWRLPLLRPPPGSVRSASCQPI | 155 |
| | RKVLVANRGEIAIRVFRACTELGLRTVAVYSEQDTGQM | |
| | HRQKADEAYLVGRGLPPVQAYLHVPDIIRVARENAVD | |
| | AIHPGYGFLSERADFAQACVDAGVRFVGPPPEVVRKM | |
| | GDKVEARSIAIAAGVPVVPGTSAPVATLGEAQDFAARV | |
| | GFPIIFKAAHGGGGRGMRAVRGPQELEESFSRASSEA | |
| | LAAFGDGALFVEKLMERPRHIEGQILGDKHGNVVHL | |
| | YERDCSIQRRHQKVVEIAPAARLDPQLRAQLASDAVRI | |
| | AQQVGYENAGTVEFLVDRDGKHYFIEVNSRLQVEHT | |
| | VTEEITGVDLVQAQLLVAAGRSLSELGLQQDSVRVNG | |
| | CAIQCRVTTEDPARGFQPDTGRIEVFRSGEGMGIRLD | |
| | GASAFQGALISPHYDSLLVKVIAHGPDQPSAAAKMSR | |
| | ALGEFRIRGVKTNIPFLQNVLAHPQFLGGAVDTQFIDE | |
| | NPELFHLRPSQNRAQKLLHYLGHVMVNGPSTPLPVK | |
| | AKAAVVEPVPPPVPMGSPPEGLRAVLQREGPAGFARA | |
| | LRGHRGLLLXDTTFRDAHQSLLATRVRTRDLARIAPFV | |
| | AHSLSPLCSMETWGGATFDVAMRFLHECPWERLREL | |
| | RRLVPNIPFQMLLRGANAVGYTNYPDNVIYRFCEVAA | |
| | ANGMDIFRIFDALNYLPNLLLGVEAVGRAGAVVEAAL | |
| | SYTGDVADPTRTKYSLDYYLGLAKELVAAGTHILCIKD | |
| | MAGLLTPAAARLLVSSLRDRFPDVPIHVHTHDTAGAAI | |
| | ATLLAAANADADVVDVAVDAMSGMTSQPSMGALVAC | |
| | ARGTPLDTGIALERVFEYSEYWEGARGLYAAFDCTAT | |
| | MKSGNADVYENEIPGGQYTNLHFQAHAMGLGHKFK | |
| | EVKKAYAEANKLLGDLIKVTPSSKVVGDLAQFMVQN | |
| | GLSREEAEARADELSFPLSVVEFLQGYIGTPPGGFPEP | |
| | FRSKVLKDLPRVEGRPGASLPPLDFEALSQELGARDG | |
| | TPPSPEDLLSAALYPKVYAEFRDFTSTFGPVSCLGTRL | |
| | FLEGPTIAEEFEVELERGKTLHIKALALGDLNAAGQR | |
| | EAFFELNGQLRSILVRDTQALKEMHVHPKADRSAKG | |
| | QVGAPMPGEVVEVRVKEGEAVEKGAPLCVLSAMKME | |
| | TVVTAPRGGTVSRLHVRPGMSLEGDDLIAEIE | |

TABLE 50

| | | SEQ |
|---|---|---|
| | | ID |
| | Sequence | NO: |
| EcoMDH-F | 5′-ACTTTTTCAAACATGAAAGTCGCAGTCCTCGG-3′ | 161 |
| EcoMDH-R | 5′-ATCATCATCATCCTTGTAATCCTTATTAACGAACTC | 162 |
| | TTCGCCC-3′ | |
| EcoMDH | MKVAVLGAAGGIGQALALLLKTQLPSGSELSLYDIAPV | 156 |
| | TPGVAVDLSHIPTAVKIKGFSGEDATPALEGADVVLISA | |
| | GVARKPGMDRSDLFNVNAGIVKNLVQQVAKTCPKACI | |
| | GIITNPVNTTVAIAAEVLKKAGVYDKNKLFGVTTLDII | |
| | RSNTFVAELKGKQPGEVEVPVIGGHSGVTILPLLSQVP | |
| | GVSFTEQEVADLTKRIQNAGTEVVEAKAGGGSATLSM | |
| | GQAAARFGLSLVRALQGEQGVVECAYVEGDGQYARFF | |
| | SQPLLLGKNGVEERKSIGTLSAFEQNALEGMLDTLKK | |
| | DIALGEEFVNK | |

The name and host of each of the manufactured transformants and the name of the introduced foreign genes are shown in Table 51.

TABLE 51

| Name | | |
|---|---|---|
| of transformant strain | Name of host strain | Introduced foreign genes |
| 42 | IGF836 | EcoPCK |
| 43 | IGF836 | GglPYC |
| 44 | IGF836 | EcoMDH |

<Confirmation of Expression of PCK, PYC, or MDH>

For 3 kinds of transformant into which only the PCK gene, the PYC gene, or the MDH gene was introduced, an expression amount of PCK, PYC, or MDH was confirmed. Specifically, each of the transformants was shake-cultured for 44 hours in 20 mL of a YPD6 medium, and the grown microbial cells were then collected and pulverized using a multibead shocker. The obtained microbial cell lysate was purified according to a protocol by using ANTI-FLAG (registered trademark) M1 Agarose Affinity Gel (manufactured by Sigma-Aldrich Co. LLC.). SDS-PAGE was performed using a purified enzyme liquid. As a result, no band was detected in any of the enzyme liquids. This result shows that even if the EcoPCK gene, the GglPYC gene, or the EcoMDH gene is introduced into S. pombe, these enzymes are not expressed, and hence effective activity is not obtained.

### Example 7 Preparation of Malic Acid Production Strain

According the method described in Examples 2, 3, and 5, transformants shown in Table 52 were prepared.

TABLE 52

| Name of | Name of | Name of introduced foreign genes |
|---|---|---|
| transformant strain | host strain | or name of detected genes |
| ASP5127 | IGF836 | Δmae2 |
| ASP5125 | IGF836 | ScePYC, Δmae2 |
| ASP5126 | IGF836 | DacMDH, Δmae2 |
| ASP5087 | IGF836 | ScePCK, DacMDH, Δmae2 |
| ASP5088 | IGF836 | PlePCK, DacMDH, Δmae2 |
| ASP5089 | IGF836 | YbePCK, DacMDH, Δmae2 |
| ASP5132 | IGF836 | KlaPYC, DacMDH, Δmae2 |
| ASP5135 | IGF836 | SpoPYC, DacMDH, Δmae2 |
| ASP5215 | IGF836 | CliMDH, ScePYC, Δmae2 |
| ASP5216 | IGF836 | HelMDH, ScePYC, Δmae2 |
| ASP5217 | IGF836 | SpuMDH, ScePYC, Δmae2 |
| ASP5129 | IGF836 | CliMDH, ScePYC, Δmae2 |
| ASP5131 | IGF836 | SpuMDH, ScePYC, Δmae2 |
| ASP5235 | IGF836 | ScePYC, LelPYC, DacMDH, HelMDH, |
| | | Δmae2 |

### Example 8 Confirmation of Effect Resulting from Deletion of Mae2

In order to confirm the effect resulting from the deletion of mae2, fermentative production of malic acid was performed using the ASP4491 strain, the ASP4964 strain, the ASP4933 strain, and the ASP5127 strain.

Specifically, the microbial cells were seeded into a YES plate and cultured for 96 hours at 30° C., thereby obtaining a colony. The obtained colony was subcultured in 5 mL of a YES medium (test tube) and shake-cultured for 24 hours at 30° C. The obtained microbial solution was subcultured in 100 mL of a YPD6 medium (Sakaguchi flask) and shake-cultured for 44 hours at 30° C.

The microbial cells obtained in this way were collected, added to 3 mL of a fermentation medium (100 g/L glucose, 111 g/L calcium carbonate) in the test tube so as to yield 36 g (weight of dry microbial cell)/L, and fermented at 30° C. under shaking conditions. A sample was collected in time from the fermented liquid.

For the obtained sample, a glucose concentration and an ethanol concentration were measured using a biosensor BF-7 (manufactured by Oji Scientific Instruments) based on an enzyme electrode method, and a malic acid concentration was measured by HPLC. During the HPLC measurement, a high-performance liquid chromatograph Prominence (manufactured by Shimadzu Corporation) was used, Aminex HPX-87H 300×7.8 mm (manufactured by Bio-Rad Laboratories, Inc.) was used as a column, an injection volume was set to be 10 μL, 10 mM H₂SO₄ was used as a solvent, a flow rate was set to be 0.6 mL/min, a measurement time was set to be 35 minutes, a measurement temperature was set to be 60° C., and diode array detector (210 nm) and a differential refractometric detector were used for detection. Each concentration is a concentration per culture solution or fermented liquid. The results are shown in Table 53.

TABLE 53

| | Fermen- | | | |
|---|---|---|---|---|
| | tation | Glucose | Ethanol | Malic acid |
| | time | concentration | concentration | concentration |
| Strain name | [hour] | [g/L] | [g/L] | [g/L] |
| ASP4491 | 0 | 100.0 | 0.0 | 0.0 |
| | 1 | 33.2 | 21.1 | 2.9 |
| | 3 | 0.0 | 34.3 | 0.0 |
| | 20 | 0.0 | 24.7 | 0.0 |
| ASP4964 | 0 | 100.0 | 0.0 | 0.0 |
| | 1 | 26.6 | 13.8 | 19.0 |
| | 3 | 0.4 | 19.2 | 26.7 |
| | 20 | 0.3 | 8.0 | 28.1 |
| ASP4933 | 0 | 100.0 | 0.0 | 0.0 |
| | 1 | 57.9 | 10.6 | 0.0 |
| | 3 | 11.8 | 27.2 | 3.0 |
| | 20 | 0.0 | 23.4 | 0.0 |
| ASP5127 | 0 | 100.0 | 0.0 | 0.0 |
| | 1 | 27.0 | 23.3 | 2.0 |
| | 2 | 0.1 | 32.9 | 3.0 |
| | 31 | 0.1 | 0.0 | 5.3 |

As a result, in the ASP4964 strain from which mae2 was deleted, the amount of malic acid produced further increased by about 10 fold than in the ASP4491 strain. In contrast, in the ASP4933 strain obtained by deleting fum1, which uses malic acid as a matrix just like mae2, from the ASP4491 strain, the amount of malic acid produced practically did not increase. Furthermore, in the ASP5127 strain from which only mae2 and pdc2 were deleted and into which PYC and MDH were not introduced, the production of malic acid in an amount of about 5 g/L was confirmed.

### Example 9 Confirmation of Effect Resulting from Introduction of PCK

In order to confirm the effect resulting from the introduction of PCK, fermentative production of malic acid was performed using the ASP5126 strain, the ASP5087 strain, the ASP5088 strain, and the ASP5089 strain.

The fermentative production of malic acid was performed in the same manner as in Example 7. The results are shown in Table 54.

TABLE 54

| | Fermen- | | | |
|---|---|---|---|---|
| | tation | Glucose | Ethanol | Malic acid |
| | time | concentration | concentration | concentration |
| Strain name | [hour] | [g/L] | [g/L] | [g/L] |
| ASP5126 | 0 | 100.0 | 0.0 | 0.0 |
| | 1 | 36.8 | 19.2 | 12.5 |
| | 2 | 3.5 | 30.8 | 19.5 |
| | 31 | 0.0 | 0.0 | 22.2 |
| ASP5087 | 0 | 100.0 | 0.0 | 0.0 |
| | 1 | 78.6 | 5.8 | 8.7 |
| | 2 | 45.9 | 12.9 | 18.5 |
| | 4 | 2.7 | 22.8 | 31.1 |
| ASP5088 | 0 | 100.0 | 0.0 | 0.0 |
| | 1 | 44.8 | 9.7 | 13.3 |
| | 3 | 0.5 | 20.4 | 22.2 |
| | 20 | 0.3 | 2.0 | 23.6 |
| ASP5089 | 0 | 100.0 | 0.0 | 0.0 |
| | 1 | 38.3 | 11.7 | 13.6 |
| | 3 | 0.3 | 20.6 | 20.4 |
| | 20 | 0.3 | 4.3 | 21.3 |

It was confirmed that in the ASP5087 strain into which PCK derived from Saccharomyces cerevisiae (ScePCK) was introduced, the ASP5088 strain into which PCK derived from Photobacterium leiognathi (PlePCK) was introduced, and the ASP5089 strain into which PCK derived from Yersinia bercovieri (YbePCK) was introduced, a malic acid production rate and a final concentration of malic acid were further improved than in the ASP5126 strain into which none of PCK and PYC were introduced.

### Example 10 Confirmation of Effect Resulting from Introduction of PYC

In order to confirm the effect resulting from the introduction of PYC, fermentative production of malic acid was performed using the ASP5126 strain, the ASP4964 strain, the ASP5132 strain, and the ASP5135 strain.

The fermentative production of malic acid was performed in the same manner as in Example 7. The results are shown in Table 55.

TABLE 55

| | Fermen- | | | |
|---|---|---|---|---|
| | tation | Glucose | Ethanol | Malic acid |
| | time | concentration | concentration | concentration |
| Strain name | [hour] | [g/L] | [g/L] | [g/L] |
| ASP5126 | 0 | 100.0 | 0.0 | 0.0 |
| | 1 | 36.8 | 19.2 | 12.5 |
| | 2 | 3.5 | 30.8 | 19.5 |
| | 31 | 0.0 | 0.0 | 22.2 |
| ASP4964 | 0 | 100.0 | 0.0 | 0.0 |
| | 1 | 20.6 | 14.1 | 26.9 |
| | 2 | 0.1 | 18.5 | 35.3 |
| | 4 | 0.0 | 14.6 | 35.1 |
| ASP5132 | 0 | 100.0 | 0.0 | 0.0 |
| | 1 | 25.9 | 8.6 | 29.7 |
| | 2 | 0.0 | 15.9 | 42.8 |
| | 4 | 0.0 | 12.0 | 42.8 |
| ASP5135 | 0 | 100.0 | 0.0 | 0.0 |
| | 1 | 41.6 | 6.5 | 27.8 |
| | 2 | 9.6 | 13.0 | 50.4 |
| | 4 | 0.4 | 14.0 | 54.5 |

It was confirmed that in the ASP4964 strain into which PYC derived from Saccharomyces cerevisiae was introduced, the ASP5132 strain into which PYC derived from Kluyveromyces lactis was introduced (KlaPYC), and the ASP5135 strain into which PYC derived from S. pombe (SpoPYC) was introduced, a malic acid production rate and a final concentration of malic acid were further improved than in the ASP5126 strain into which none of PCK and PYC were introduced.

### Example 11 Confirmation of Effect Resulting from Introduction of MDH

In order to confirm the effect resulting from the introduction of MDH, fermentative production of malic acid was performed using the ASP5125 strain, the ASP5215 strain, the ASP5216 strain, the ASP5127 strain, the ASP4964 strain, the ASP5129 strain, and the ASP5131 strain.

The fermentative production of malic acid was performed in the same manner as in Example 7. The results are shown in Table 56.

TABLE 56

| | Fermen- | | | |
|---|---|---|---|---|
| | tation | Glucose | Ethanol | Malic acid |
| | time | concentration | concentration | concentration |
| Strain name | [hour] | [g/L] | [g/L] | [g/L] |
| ASP5125 | 0 | 100.0 | 0.0 | 0.0 |
| | 1 | 36.6 | 20.4 | 0.2 |
| | 2 | 4.6 | 33.6 | 0.5 |
| | 31 | 0.1 | 0.0 | 3.2 |
| ASP5215 | 0 | 100.0 | 0.0 | 0.0 |
| | 1 | 69.8 | 8.8 | 13.5 |
| | 2 | 34.8 | 17.0 | 24.5 |
| | 4 | 0.0 | 24.6 | 32.6 |
| ASP5216 | 0 | 100.0 | 0.0 | 0.0 |
| | 1 | 73.7 | 8.3 | 13.8 |
| | 2 | 37.7 | 16.7 | 24.9 |
| | 4 | 0.0 | 23.4 | 37.1 |
| ASP5217 | 0 | 100.0 | 0.0 | 0.0 |
| | 1 | 71.0 | 9.0 | 12.9 |
| | 2 | 34.9 | 18.8 | 24.2 |
| | 4 | 0.0 | 23.7 | 35.1 |
| ASP4964 | 0 | 100.0 | 0.0 | 0.0 |
| | 1 | 28.6 | 13.7 | 24.0 |
| | 3 | 0.3 | 18.2 | 35.5 |
| | 6 | 0.1 | 13.7 | 37.0 |
| ASP5129 | 0 | 100.0 | 0.0 | 0.0 |
| | 1 | 24.6 | 15.9 | 25.5 |
| | 3.5 | 0.0 | 18.6 | 35.9 |
| | 24 | 0.0 | 0.0 | 47.2 |
| ASP5131 | 0 | 100.0 | 0.0 | 0.0 |
| | 1 | 29.1 | 14.2 | 26.0 |
| | 3 | 0.1 | 19.8 | 36.0 |
| | 6 | 0.1 | 13.2 | 39.5 |

It was confirmed that in the ASP5215 strain into which CliMDH and PCK were introduced, the ASP5216 strain into which HelMDH and PCK were introduced, the ASP5217 strain into which SpuMDH and PCK were introduced, the ASP4964 strain into which HelMDH and PYC were introduced, the ASP5129 strain into which CliMDH and PYC were introduced, and the ASP5131 strain into which SpuMDH and PYC were introduced, a malic acid production rate and a final concentration of malic acid were further improved than in the ASP5125 strain into which MDH was not introduced but PYC was introduced.

### Example 12 Confirmation of Effect Resulting from Increasing Number of Copies of Introduced Genes

In order to confirm the effect resulting from increasing the number of copies of the introduced genes, fermentative production of malic acid was performed using the ASP4964 strain and the ASP5235 strain.

The fermentative production of malic acid was performed in the same manner as in Example 7. The results are shown in Table 57.

TABLE 57

| | Fermen- | | | |
|---|---|---|---|---|
| | tation | Glucose | Ethanol | Malic acid |
| | time | concentration | concentration | concentration |
| Strain name | [hour] | [g/L] | [g/L] | [g/L] |
| ASP4964 | 0 | 100.0 | 0.0 | 0.0 |
| | 1 | 35.5 | 15.0 | 22.2 |
| | 2 | 0.0 | 22.6 | 36.9 |
| | 4 | 0.0 | 19.1 | 38.4 |
| ASP5235 | 0 | 100.0 | 0.0 | 0.0 |
| | 1 | 49.7 | 9.6 | 28.4 |
| | 2 | 3.7 | 19.4 | 53.6 |
| | 4 | 0.0 | 17.7 | 56.9 |

It was confirmed that in the ASP5235 strain into which 2 copies of PYC and 2 copies of MDH were introduced, a malic acid production rate and a final concentration of malic acid were further improved than in the ASP4964 strain into which one copy of PYC and one copy of MDH were introduced.

### Example 13 Fermentative Production of Malic Acid Under Non-Neutralization Condition

In order to confirm the production of malic acid under non-neutralization conditions, fermentative production of malic acid was performed using the ASP4964 strain and the ASP5235 strain.

Specifically, microbial cells grown by the same method as in Example 7 were added to 3 mL of a fermentation medium (100 g/L glucose) in a test tube so as to yield 36 g (weight of dry microbial cells)/L, and fermented at 30° C. under shaking conditions. A sample was collected in time from the fermented liquid.

The obtained sample was measured in the same manner as in Example 7. The results are shown in Table 58. As a result, it was confirmed that all of the strains produced malic acid under non-neutralization conditions. The pH of the ASP4964 strain sample that was collected after fermentation was performed for 1 hour was 3.5, and the pH of the sample that was collected after fermentation was performed for 2 hours was 3.0. Furthermore, the pH of the ASP5235 strain sample that was collected after fermentation was performed for 1 hour was 3.0, and the pH of the sample that was collected after fermentation was performed for 2 hours was 2.8.

TABLE 58

| | Fermen- | | | |
|---|---|---|---|---|
| | tation | Glucose | Ethanol | Malic acid |
| | time | concentration | concentration | concentration |
| Strain name | [hour] | [g/L] | [g/L] | [g/L] |
| ASP4964 | 0 | 100.0 | 0.0 | 0.0 |
| | 1 | 45.9 | 12.1 | 14.3 |
| | 2 | 11.5 | 20.5 | 21.5 |
| | 4 | 0.0 | 23.1 | 23.3 |
| ASP5235 | 0 | 100.0 | 0.0 | 0.0 |
| | 1 | 51.8 | 7.6 | 22.5 |
| | 2 | 26.1 | 14.7 | 33.6 |
| | 4 | 6.0 | 18.1 | 38.3 |
| | 5 | 1.2 | 17.6 | 40.7 |

Priority is claimed on Japanese Patent Application No. 2014-135043, filed on Jun. 30, 2014, the content of which is incorporated herein by reference.

What is claimed is:

1. A transformant which uses Schizosaccharomyces pombe as a host and into which one or more foreign genes of one or more kinds selected from the group consisting of a phosphoenolpyruvate carboxykinase gene and a pyruvate carboxylase gene are incorporated,
wherein some of the genes in a group of pyruvate decarboxylase-encoding genes of the Schizosaccharomyces pombe host have undergone deletion or deactivation,
the phosphoenolpyruvate carboxykinase gene encodes a polypeptide which includes an amino acid sequence represented by any of SEQ ID NOS: 94 to 113, a polypeptide which includes an amino acid sequence obtained by the substitution, addition, or deletion of one or a plurality amino acids in an amino acid sequence represented by any of SEQ ID NOS: 94 to 113 and has a phosphoenolpyruvate carboxykinase activity, or a polypeptide which includes an amino acid sequence sharing a sequence identity of equal to or higher than 80% with an amino acid sequence represented by any of SEQ ID NOS: 94 to 113 and has phosphoenolpyruvate carboxykinase activity,
the pyruvate carboxylase gene encodes a polypeptide which includes an amino acid sequence represented by any of SEQ ID NOS: 1 to 16, a polypeptide which includes an amino acid sequence obtained by the substitution, addition, or deletion of one or a plurality of amino acids in an amino acid sequence represented by any of SEQ ID NOS: 1 to 16 and has pyruvate carboxylase activity, or a polypeptide which includes an amino acid sequence sharing a sequence identity of equal to or higher than 80% with an amino acid sequence represented by any of SEQ ID NOS: 1 to 16 and has pyruvate carboxylase activity, and
the pyruvate decarboxylase-encoding genes that have undergone deletion or deactivation are pdc2 genes.

2. The transformant according to claim 1 into which one or more foreign malate dehydrogenase genes are further incorporated.

3. The transformant according to claim 2,
wherein the malate dehydrogenase genes encode a polypeptide which includes an amino acid sequence represented by any of SEQ ID NOS: 17 to 21, a polypeptide which includes an amino acid sequence obtained by the substitution, addition, or deletion of one or a plurality of amino acids in an amino acid sequence represented by any of SEQ ID NOS: 17 to 21 and has malate dehydrogenase activity, or a polypeptide which includes an amino acid sequence sharing a sequence identity of equal to or higher than 80% with an amino acid sequence represented by any of SEQ ID NOS: 17 to 21 and has malate dehydrogenase activity.

4. The transformant according to any one of claims 1 to 3,
wherein malic enzyme genes have also undergone deletion or deactivation.

5. The transformant according to claim 1 into which one or more foreign phosphoenolpyruvate carboxykinase genes or one or more foreign pyruvate carboxylase genes and one or more foreign malate dehydrogenase genes are incorporated,
wherein all of the pdc2 genes and the malic enzyme genes have undergone deletion or deactivation.

6. The transformant according to any one of claims 1 to 5,
wherein the foreign genes are incorporated into a chromosome of the host.

7. A method for manufacturing a dicarboxylic acid having 4 carbon atoms, comprising:
culturing the transformant according to any one of claims 1 to 6 in a culture solution; and
obtaining a dicarboxylic acid having 4 carbon atoms from the cultured transformant or a culture supernatant.

8. The method for manufacturing a dicarboxylic acid having 4 carbon atoms according to claim 7,
wherein the dicarboxylic acid having 4 carbon atoms is malic acid or oxaloacetic acid.

9. The method for manufacturing a dicarboxylic acid having 4 carbon atoms according to claim 7 or 8,
wherein the culturing is continued even after a pH of the culture solution becomes equal to or less than 3.5.
